(19) United States
(12) Patent Application Publication
(10) Pub. No.: US 2007/0225167 A1
(43) Pub. Date: Sep. 27, 2007

(54) 2-Ethyl-4,6-Dimethyl-Phenyl-Substituted Tetramic Acid Derivatives as Pest Control Agents and/or Herbicides

(75) Inventors: Reiner Fischer, Monheim (DE); Stefan Lehr, Liederbach (DE); Dieter Feucht, Eschborn (DE); Peter Losel, Leverkusen (DE); Olga Malsam, Rosrath (DE); Guido Bojack, Wiesbaden (DE); Thomas Auler, Leichlingen (DE); Martin Jeffrey Hills, Idstein (DE); Heinz Kehne, Hofheim (DE); Christopher Hugh Rosinger, Hofheim (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer Cropscience Aktiengesellschaft, Monheim (DE)

(21) Appl. No.: 10/579,099

(22) PCT Filed: Nov. 9, 2004
(86) PCT No.: PCT/EP04/12646

§ 371 Date: Mar. 22, 2007

(30) Foreign Application Priority Data
Nov. 22, 2003 (DE) 10354628.6

(51) Int. Cl. A01N 43/36; C07D 207/38

(52) U.S. Cl. 504/105; 504/109; 504/247; 504/283; 514/312; 514/378; 514/383; 514/425; 546/152; 548/240; 548/262.2; 548/544

(57) ABSTRACT

The invention relates to a novel 2-ethyl-4,6-dimethylphenyl-substituted tetramic acid derivatives of the formula (I) 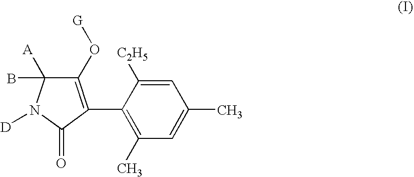 in which A, B, D and G are as defined above, to a plurality of processes and intermediates for the preparation and to their use as pesticides and/or herbicides, and also to selective herbicidal compositions comprising firstly 2-ethyl-4,6-dimethylphenyl-substituted tetramic acid derivatives of the formula (I) or (I-a′) and secondly at least one crop plant compatibility-improving compound.

The invention relates to novel 2-ethyl-4,6-dimethylphenyl-substituted tetramic acid derivatives, to a plurality of processes and intermediates for their preparation and to their use as pesticides and/or herbicides.

Moreover, the invention relates to novel selective herbicidal active compound combinations comprising both the 2-ethyl-4,6-dimethylphenyl-substituted spirocyclic tetramic acid derivatives and at least one crop plant compatibility-improving compound, which combinations can be used with particularly good results for the selective control of weeds in various crops of useful plants.

3-Acylpyrrolidine-2,4-diones are described as having pharmaceutical properties (S. Suzuki et al. Chem. Pharm. Bull. 15 1120 (1967)). Furthermore, N-phenylpyrrolidine-2,4-diones were synthesized by R. Schmierer and H. Mildenberger (Liebigs Ann. Chem. 1985, 1095). A biological activity of these compounds has not been described.

EP-A-0 262 399 and GB-A-2 266 888 disclose compounds of a similar structure (3-arylpyrrolidine-2,4-diones); however, a herbicidal, insecticidal or acaricidal action of these compounds has hitherto not been described. Unsubstituted bicyclic 3-arylpyrrolidine-2,4-dione derivatives (EP-A-355 599 and EP-A-415 211) and substituted monocyclic 3-arylpyrrolidine-2,4-dione derivatives (EP-A-377 893 and EP-A-442 077) having herbicidal, insecticidal or acaricidal action are known.

Also known are polycyclic 3-arylpyrrolidine-2,4-dione derivatives (EP-A442 073) and 1H-aryl-pyrrolidinedione derivatives (EP-A-456 063, EP-A-521 334, EP-A-596 298, EP-A-613 884, EP-A-613 885, WO 94/01 997, WO 95/26954, WO 95/20 572, EP-A 0 668 267, WO 96/25 395, WO 96 35 664, WO 97/01 535, WO 97/02 243, WO 97/36 868, WO 97/43275, WO/98/05638, WO 98/06721, WO 98/25928, WO 99/16748, WO 99/24437, WO 99/43649, WO 99/48869, WO 99/55673, WO 01/09092, WO 01/17972, WO 01/23354, WO 01/74770 and WO 03/013249).

However, in particular at low application rates and concentrations, the activity and the activity spectrum of these compounds are not always entirely satisfactory. Moreover, the compatibility with plants of these compounds is not always sufficient.

This invention now provides novel compounds of the formula (I) 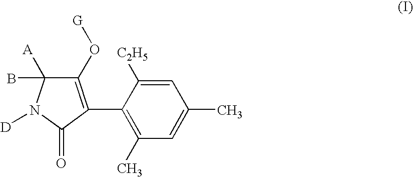
in which
G represents one of the groups 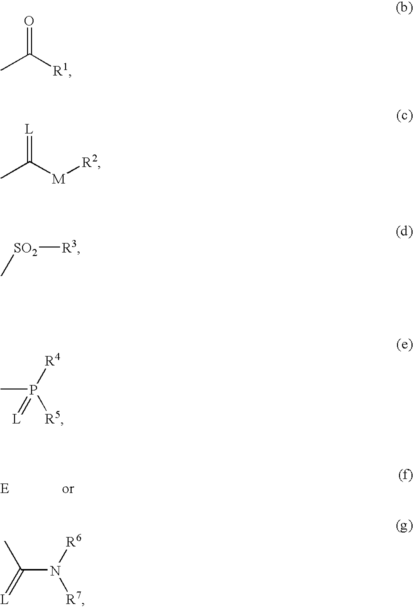
in which
   E represents a metal ion equivalent or an ammonium ion,
   L represents oxygen or sulphur,
   M represents oxygen or sulphur,
R¹ represents in each case optionally substituted alkyl, alkenyl, alkoxyalkyl, alkylthioalkyl or polyalkoxyalkyl or represents in each case optionally halogen-, alkyl-, or alkoxy-substituted cycloalkyl or heterocyclyl or represents in each case optionally a substituted phenyl, phenylalkyl, phenylalkenyl or heteroaryl,
R² represents in each case optionally halogen, substituted alkyl, alkenyl, alkoxyalkyl or polyalkoxyalkyl or represents in each case optionally substituted cycloalkyl, phenyl or benzyl,
R³, R⁴ and R⁵ independently of one another represent in each case optionally halogen-substituted alkyl, alkoxy, alkylamino, dialkylamino, alkylthio, alkenylthio or cycloalkylthio or represent in each case optionally substituted phenyl, benzyl, phenoxy or phenylthio,
R⁶ and R⁷ independently of one another represent hydrogen, represent in each case optionally halogen-substituted alkyl, cycloalkyl, alkenyl, alkoxy, alkoxyalkyl, represent in each case optionally substituted phenyl or benzyl or together with the N atom to which they are attached form an optionally substituted cycle which optionally contains oxygen or sulphur,
A represents hydrogen, represents in each case optionally halogen-substituted alkyl, alkenyl, alkoxyalkyl or alkylthioalkyl or represents optionally substituted cycloalkyl,
B represents hydrogen, alkyl or alkoxyalkyl,
D represents hydrogen or represents an optionally substituted radical from the group consisting of alkyl, alkenyl, alkynyl, alkoxyalkyl, alkylthioalkyl, or optionally substituted cycloalkyl,
A and D together with the atoms to which they are attached, represent a saturated or unsaturated cycle which optionally contains at least one heteroatom and which is unsubstituted or substituted in the A,D moiety, and, if
G represents hydrogen (a), then
A represents hydrogen or alkyl,
B represents hydrogen or alkyl,
D represents an optionally substituted radical from the group consisting of alkyl, alkenyl, alkynyl, alkoxyalkyl, alkylthioalkyl, or optionally substituted cycloalkyl, or
A and D together with the atoms to which they are attached represent a saturated or unsaturated cycle which optionally contains at least one heteroatom and which is unsubstituted or substituted in the A,D moiety.

Depending inter alia on the nature of the substituents, the compounds of the formula (I) may be present as geometrical and/or optical isomers or isomer mixtures of varying composition which, if desired, can be separated in a customary manner. The present invention provides both the pure isomers and the isomer mixtures, their preparation and use and compositions comprising them. However, for the sake of simplicity, hereinbelow only compounds of the formula (I) are referred to, although what is meant are both the pure compounds and, if appropriate, mixtures having varying proportions of isomeric compounds.

Including the different meanings (a), (b), (c), (d), (e), (f) and (g) of group G, the following principle structures (I-a) to (I-g) result: 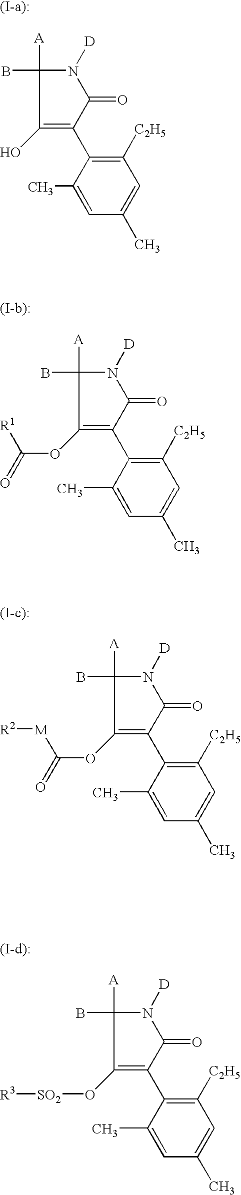 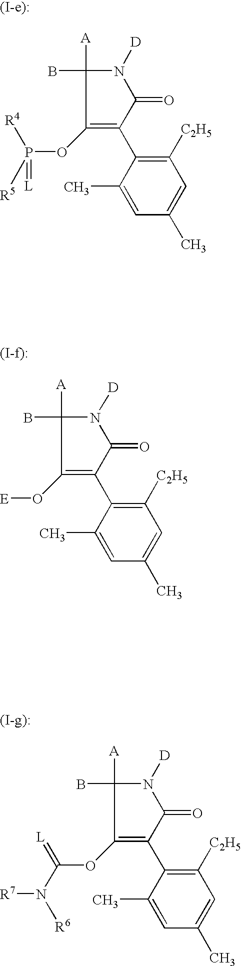
in which
A, B, D, E, L, M, R¹, R², R³, R⁴, R⁵, R⁶ and R⁷ are as defined above.

Furthermore, it has been found that the novel compounds of the formula (I) are obtained by one of the processes described below:
(A) Compounds of the formula (I-a), 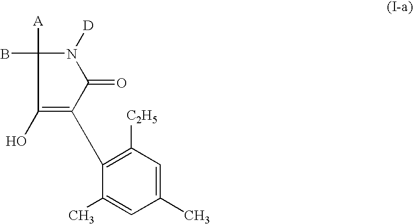
in which
   A, B and D are as defined above, are obtained when compounds of the formula (II), 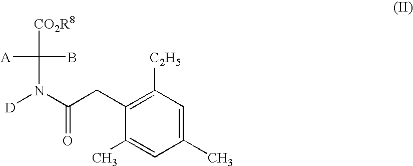
   in which
   A, B and D are as defined above and
   R⁸ represents alkyl (preferably C₁-C₆-alkyl),
   are condensed intramolecularly in the presence of a diluent and in the presence of a base.
(B) Compounds of the formula (I-b) shown above in which A, B, D and R¹ are as defined above are obtained when compounds of the formula (I-a) shown above or of the formula (I-a′) shown on p. 10 in which A, B and D are each as defined above are reacted
α) with acid halides of the formula (III), 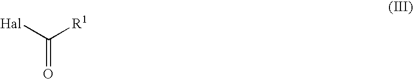
in which
   R¹ is as defined above and
   Hal represents halogen (in particular chlorine or bromine) or
β) with carboxylic anhydrides of the formula (IV),
   R¹—CO—O—CO—R¹ (IV)
in which
   R¹ is as defined above,
   if appropriate in the presence of a diluent and if appropriate in the presence of an acid binder.
(C) Compounds of the formula (I-c) show above in which A, B, D, R² and M are as defined above and L represents oxygen are obtained when compounds of the formula (I-a) shown above or of the formula (I-a′) shown on p. 10 in which A, B and D are each as defined above are in each case reacted
   with chloroformic esters or chloroformic thioesters of the formula (V),
      R²-M-CO—Cl (V)
   in which
   R² and M are as defined above,
   if appropriate in the presence of a diluent and if appropriate in the presence of an acid binder.
(D) Compounds of the formula (I-c) shown above in which A, B, D, R² and M are as defined above and L represents sulphur are obtained when compounds of the formula (I-a) shown above or of the formula (I-a′) shown on p. 10 in which A, B and D are each as defined above are in each case reacted
α) with chloromonothioformic esters or chlorodithioformic esters of the formula (VI) 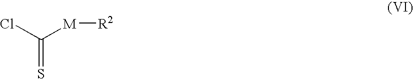
in which
   M and R² are as defined above,
   if appropriate in the presence of a diluent and if appropriate in the presence of an acid binder, or
β) with carbon disulfide and then with compounds of the formula (VII)
   R²-Hal (VII)
in which
   R² is as defined above and
   Hal represents chlorine, bromine or iodine,
   if appropriate in the presence of a diluent and if appropriate in the presence of base.
(E) Compounds of the formula (I-d) show above in which A, B, D and R³ are as defined above are obtained when compounds of the formula (I-a) shown above or of the formula (I-a′) shown on p. 10 in which A, B and D are each as defined above are in each case reacted with sulfonyl chlorides of the formula (VIII),
   R³—SO₂—Cl (VIII)
in which
   R³ is as defined above,
   if appropriate in the presence of a diluent and if appropriate in the presence of an acid binder.
(F) Compounds of the formula (I-e) shown above in which A, B, D, L, R⁴ and R⁵ are as defined above are obtained when compounds of the formula (I-a) shown above or of the formula (I-a′) shown on p. 10 in which A, B and D are each as defined above are in each case reacted
   with phosphorus compounds of the formula (IX), 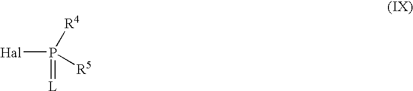
   in which
   L, R⁴ and R⁵ are as defined above and
   Hal represents halogen (in particular chlorine or bromine),
   if appropriate in the presence of a diluent and if appropriate in the presence of an acid binder.
(G) Compounds of the formula (I-f) shown above in which A, B, D and E are as defined above are obtained when compounds of the formula (I-a) shown above or of the formula (I-a′) shown on p. 10 in which A, B and D are as defined above are in each case reacted
   with metal compounds or amines of the formulae (X) or (XI), respectively, 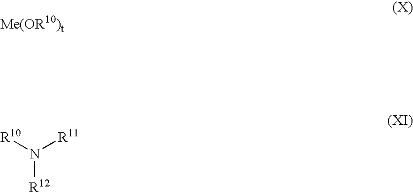
   in which
   Me represents a mono- or divalent metal (preferably an alkali metal or alkaline earth metal, such as lithium, sodium, potassium, magnesium or calcium),
   t represents the number 1 or 2 and
   R¹⁰, R¹¹, R¹² independently of one another represent hydrogen or alkyl (preferably C₁-C₈-alkyl),
   if appropriate in the presence of a diluent.
(H) Compounds of the formula (I-g) shown above in which A, B, D, L, R⁶ and R⁷ are as defined above are obtained when compounds of the formula (I-a) shown above or of the formula (I-a′) shown on p. 10 in which A, B and D are as defined above are in each case reacted
α) with isocyanates or isothiocyanates of the formula (XII),
   R⁶—N═C=L (XII)
in which
   R⁶ and L are as defined above,
   if appropriate in the presence of a diluent and if appropriate in the presence of a catalyst; or
β) with carbamoyl chlorides or thiocarbamoyl chlorides of the formula (XIII), 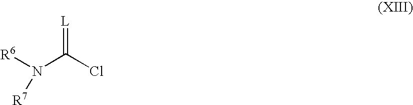
in which
   L, R⁶ and R⁷ are as defined above,
   if appropriate in the presence of a diluent and if appropriate in the presence of an acid binder.

Furthermore, it has been found that the novel compounds of the formula (I) are highly active pesticides, preferably insecticides and/or acaricides, and/or herbicides.

Surprisingly, it has now also been found that certain substituted cyclic ketoenols, when used together with crop plant capability-improving compounds (safeners/antidotes) described below, are highly suitable for preventing damage to the crop plant and can be used particularly advantageously as broad-spectrum effective combination preparations for the selective control of unwanted plants in crops of useful plants, such as, for example, in cereals, but also maize, soya beans and rice.

The invention also provides selective herbicidal compositions comprising an effective amount of a combination of active compounds comprising the components
a′) at least one substituted cyclic ketoenol of the formula (I) in which A, B, D and G are as defined above or
b′) at least one substituted cyclic ketoenol of the formula (I-a′) 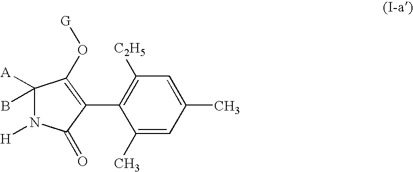
in which
   G represents hydrogen (a),
   A represents hydrogen, represents in each case optionally halogen-substituted alkyl, alkenyl, alkoxyalkyl or alkylthioalkyl or represents optionally substituted cycloalkyl,
   B represents hydrogen, alkyl or alkoxyalkyl and
c′) at least one crop plant compatibility-improving compound from the following group of compounds:
   4-dichloroacetyl-1-oxa-4-azaspiro[4.5]decane (AD-67, MON4660), 1-dichloroacetylhexahydro-3,3,8a-trimethylpyrrolo[1,2-a]pyrimidin-6(2H)-one (dicyclonon, BAS-145138), 4-dichloroacetyl-3,4-dihydro-3-methyl-2H-1,4-benzoxazine (benoxacor), 1-methylhexyl 5-chloroquinoline-8-oxyacetate (cloquintocet-mexyl—cf. also related compounds in EP-A-86750, EP-A-94349, EP-A-191736, EP-A-492366), 3-(2-chlorobenzyl)-1-(1-methyl-1-phenylethyl)urea (cumyluron), α-(cyanomethoximino)phenylacetonitrile (cyometrinil), 2,4-dichlorophenoxyacetic acid (2,4-D), 4-(2,4-dichlorophenoxy)butyric acid (2,4-DB), 1-(1-methyl-1-phenylethyl)-3-(4-methylphenyl)urea (daimuron, dymron), 3,6-dichloro-2-methoxybenzoic acid (dicamba), S-1-methyl 1-phenylethyl piperidine-1-thiocarboxylate (dimepiperate), 2,2-dichloro-N-(2-oxo-2-(2-propenylamino)ethyl)-N-(2-propenyl)acetamide (DKA-24), 2,2-dichloro-N,N-di-2-propenylacetamide (dichlormid), 4,6-dichloro-2-phenylpyrimidine (fenclorim), ethyl 1-(2,4-dichlorophenyl)-5-trichloromethyl-1H-1,2,4-triazole-3-carboxylate (fenchlorazole-ethyl—cf. also related compounds in EP-A-174562 and EP-A-346620), phenylmethyl 2-chloro-4-trifluoromethylthiazole-5-carboxylate (flurazole), 4-chloro-N-(1,3-dioxolan-2-ylmethoxy)-α-trifluoroacetophenone oxime (fluxofenim), 3-dichloro-acetyl-5-(2-furanyl)-2,2-dimethyloxazolidine (furilazole, MON-13900), ethyl 4,5-dihydro-5,5-diphenyl-3-isoxazolecarboxylate (isoxadifen-ethyl—cf. also related compounds in WO-A-95/07897), 1-(ethoxycarbonyl)ethyl 3,6-dichloro-2-methoxybenzoate (lactidichlor), (4-chloro-o-tolyloxy)acetic acid (MCPA), 2-(4-chloro-o-tolyloxy)propionic acid (mecoprop), diethyl 1-(2,4-dichorophenyl)-4,5-dihydro-5-methyl-1H-pyrazole-3,5-dicarboxylate (mefenpyr-diethyl—cf. also related compounds in WO-A-91/07874), 2-dichloromethyl-2-methyl-1,3-dioxolane (MG-191), 2-propenyl-1-oxa-4-azaspiro[4.5]decane-4-carbodithioate (MG-838), 1,8-naphthalic anhydride, α-(1,3-dioxolan-2-ylmethoximino)phenylacetonitrile (oxabetrinil), 2,2-dichloro-N-(1,3-dioxolan-2-ylmethyl)-N-(2-propenyl)acetamide (PPG-1292), 3-dichloroacetyl-2,2-dimethyloxazolidine (R-28725), 3-dichloroacetyl-2,2,5-trimethyloxazolidine (R-29148), 4-(4-chloro-o-tolyl)butyric acid, 4-(4-chlorophenoxy)butyric acid, diphenylmethoxyacetic acid, methyl diphenylmethoxyacetate, ethyl diphenylmethoxyacetate, methyl 1-(2-chlorophenyl)-5-phenyl-1H-pyrazole-3-carboxylate, ethyl 1-(2,4-dichlorophenyl)-5-methyl-1H-pyrazole-3-carboxylate, ethyl 1-(2,4-dichlorophenyl)-5-isopropyl-1H-pyrazole-3-carboxylate, ethyl 1-(2,4-dichlorophenyl)-5-(1,1-dimethylethyl)-1H-pyrazole-3-carboxylate, ethyl 1-(2,4-dichlorophenyl)-5-phenyl-1H-pyrazole-3-carboxylate (cf. also related compounds in EP-A-269806 and EP-A-333131), ethyl 5-(2,4-dichlorobenzyl)-2-isoxazoline-3-carboxylate, ethyl 5-phenyl-2-isoxazoline-3-carboxylate, ethyl 5-(4-fluorophenyl)-5-phenyl-2-isoxazoline-3-carboxylate (cf. also related compounds in WO-A-91/08202), 1,3-dimethylbut-1-yl 5-chloroquinoline-8-oxyacetate, 4-allyloxybutyl 5-chloroquinoline-8-oxyacetate, 1-allyloxyprop-2-yl 5-chloroquinoline-8-oxyacetate, methyl 5-chloroquinoxaline-8-oxyacetate, ethyl 5-chloroquinoline-8-oxyacetate, allyl 5-chloroquinoxaline-8-oxyacetate, 2-oxoprop-1-yl 5-chloroquinoline-8-oxyacetate, diethyl 5-chloroquinoline-8-oxymalonate, diallyl 5-chloroquinoxaline-8-oxymalonate, diethyl 5-chloroquinoline-8-oxymalonate (cf. also related compounds in EP-A-582198), 4-carboxychroman-4-ylacetic acid (AC-304415, cf. EP-A-613618), 4-chlorophenoxyacetic acid, 3,3′-dimethyl-4-methoxybenzophenone, 1-bromo-4-chloromethylsulphonylbenzene, 1-[4-(N-2-methoxybenzoylsulphamoyl)phenyl]-3-methylurea (also known as N-(2-methoxybenzoyl)-4-[(methylaminocarbonyl)amino]benzenesulphonamide), 1-[4-(N-2-methoxybenzoylsulphamoyl)phenyl]-3,3-dimethylurea, 1-[4-(N-4,5-dimethylbenzoyl-sulphamoyl)phenyl]-3-methylurea, 1-[4-(N-naphthylsulphamoyl)phenyl]-3,3-dimethylurea, N-(2-methoxy-5-methylbenzoyl)-4-(cyclopropylaminocarbonyl)benzenesulphonamide,
   and/or one of the following compounds, defined by general formulae,
   of the general formula (IIa) 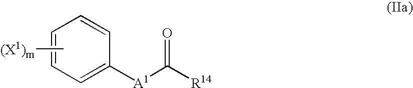
   or of the general formula (IIb) 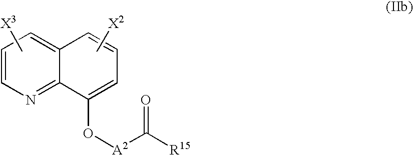
   or of the formula (IIc) 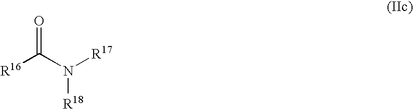
   where
m represents the number 0, 1, 2, 3, 4 or 5,
A¹ represents one of the divalent heterocyclic groupings shown below, 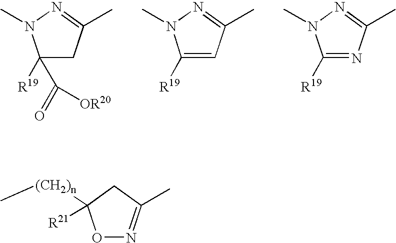
n represents a number between 0 and 5,
A² represents optionally C₁-C₄-alkyl- and/or C₁-C₄-alkoxy-carbonyl- and/or C₁-C₄-alkenyloxy-carbonyl-substituted alkanediyl having 1 or 2 carbon atoms,
R¹⁴ represents hydroxyl, mercapto, amino, C₁-C₆-alkoxy, C₁-C₆-alklylthio, C₁-C₆-alkylamino or di-(C₁-C₄-alkyl)amino,
R¹⁵ represents hydroxyl, mercapto, amino, C₁-C₆-alkoxy, C₁-C₆-alkenyloxy, C₁-C₆-alkenyloxy-C₁-C₆-alkoxy, C₁-C₆-alkylthio, C₁-C₆-alkylamino or di(C₁-C₄-alkyl)amino,
R¹⁶ represents in each case optionally fluorine-, chlorine- and/or bromine-substituted C₁-C₄-alkyl,
R¹⁷ represents hydrogen, in each case optionally fluorine-, chlorine- and/or bromine-substituted C₁-C₆-alkyl, C₂-C₆-alkenyl or C₂-C₆-alkynyl, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or optionally fluorine-, chlorine- and/or bromine- or C₁-C₄-alkyl-substituted phenyl,
R¹⁸ represents hydrogen, in each case optionally fluorine-, chlorine- and/or bromine-substituted C₁-C₆-alkyl, C₂-C₆-alkenyl or C₂-C₆-alkynyl, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or optionally fluorine-, chlorine- and/or bromine- or C₁-C₄-alkyl-substituted phenyl, R¹⁷ and R¹⁸ also together represent C₃-C₆-alkanediyl or C₂-C₅-oxaalkanediyl, each of which is optionally substituted by C₁-C₄-alkyl, phenyl, furyl, a fused benzene ring or by two substituents which, together with the C atom to which they are attached, form a 5- or 6-membered carbocycle,
R¹⁹ represents hydrogen, cyano, halogen, or represents in each case optionally fluorine-, chlorine- and/or bromine-substituted C₁-C₄-alkyl, C₃-C₆-cycloalkyl or phenyl,
R²⁰ represents hydrogen, optionally hydroxyl-, cyano-, halogen- or C₁-C₄-alkoxy-substituted C₁-C₆-alkyl, C₃-C₆-Cycloalkyl or tri(C₁-C₄-alkyl)silyl,
R²¹ represents hydrogen, cyano, halogen, or represents in each case optionally fluorine-, chlorine- and/or bromine-substituted C₁-C₄-alkyl, C₃-C₆-cycloalkyl or phenyl,
X¹ represents nitro, cyano, halogen, C₁-C₄-alkyl, C₁-C₄-haloalkyl, C₁-C₄-alkoxy or C₁-C₄-haloalkoxy,
X² represents hydrogen, cyano, nitro, halogen, C₁-C₄-alkyl, C₁-C₄-haloalkyl, C₁-C₄-alkoxy or C₁-C₄-haloalkoxy,
X³ represents hydrogen, cyano, nitro, halogen, C₁-C₄-alkyl, C₁-C₄-haloalkyl, C₁-C₄-alkoxy or C₁-C₄-haloalkoxy,
   and/or the following compounds, defined by general formulae,
   of the general formula (IId) 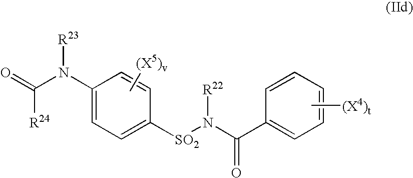
   or of the general formula (IIe) 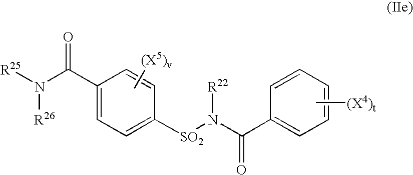
   where
t represents the number 0, 1, 2, 3, 4 or 5,
v represents the number 0, 1, 2, 3, 4 or 5,
R²² represents hydrogen or C₁-C₄-alkyl,
R²³ represents hydrogen or C₁-C₄-alkyl,
R²⁴ represents hydrogen, in each case optionally cyano-, halogen- or C₁-C₄-alkoxy-substituted C₁-C₆-alkyl, C₁-C₆-alkoxy, C₁-C₆-alkylthio, C₁-C₆-alkylamino or di(C₁-C₄-alkyl)amino, or in each case optionally cyano-, halogen- or C₁-C₄-alkyl-substituted C₃-C₆-Cycloalkyl, C₃-C₆-cycloalkyloxy, C₃-C₆-Cycloalkylthio or C₃-C₆-cycloalkylamino,
R²⁵ represents hydrogen, optionally cyano-, hydroxyl-, halogen- or C₁-C₄-alkoxy-substituted C₁-C₆-alkyl, in each case optionally cyano- or halogen-substituted C₃-C₆-alkenyl or C₃-C₆-alkynyl, or optionally cyano-, halogen- or C₁-C₄-alkyl-substituted C₃-C₆-cycloalkyl,
R²⁶ represents hydrogen, optionally cyano-, hydroxyl-, halogen- or C₁-C₄-alkoxy-substituted C₁-C₆-alkyl, in each case optionally cyano- or halogen-substituted C₃-C₆-alkenyl or C₃-C₆-alkynyl, optionally cyano-, halogen- or C₁-C₄-alkyl-substituted C₃-C₆-Cycloalkyl, or optionally nitro-, cyano-, halogen-, C₁-C₄-alkyl-, C₁-C₄-haloalkyl, C₁-C₄-alkoxy- or C₁-C₄-haloalkoxy-substituted phenyl, or together with R²⁵ represents in each case optionally C₁-C₄-alkyl-substituted C₂-C₆-alkanediyl or C₂-C₅-oxaalkanediyl,
X⁴ represents nitro, cyano, carboxyl, carbamoyl, formyl, sulphamoyl, hydroxyl, amino, halogen, C₁-C₄-alkyl, C₁-C₄-haloalkyl, C₁-C₄-alkoxy or C₁-C₄-haloalkoxy, and
X⁵ represents nitro, cyano, carboxyl, carbamoyl, formyl, sulphamoyl, hydroxyl, amino, halogen, C₁-C₄-alkyl, C₁-C₄-haloalkyl, C₁-C₄-alkoxy or C₁-C₄-haloalkoxy.

The formula (I) provides a general definition of the compounds according to the invention. Preferred substituents or ranges of the radicals listed in the formulae given above and below are illustrated below:

If G preferably represents hydrogen (a), then
A preferably represents hydrogen or C₁-C₈-alkyl,
B preferably represents hydrogen or C₁-C₆-alkyl,
D preferably represents C₁-C₈-alkyl, C₁-C₈-alkenyl, C₁-C₆-alkoxy-C₂-C₄-alkyl or C₁-C₆-alkyl-thio-C₂-C₄-alkyl, each of which is optionally mono- to pentasubstituted by halogen, represents-C₃-C₈-Cycloalkyl which is optionally mono- to trisubstituted by halogen, C₁-C₄-alkyl, C₁-C₄-alkoxy or C₁-C₂-haloalkyl,
A and D together represent a C₃-C₆-alkanediyl or C₃-C₆-alkenediyl group in which in each case optionally one methylene group is replaced by oxygen or sulphur and which are in each case optionally mono- or disubstituted by halogen, hydroxyl, C₁-C₄-alkyl or C₁-C₄-alkoxy, or by a further C₃-C₆-alkanediyl, C₃-C₆-alkenediyl or C₄-C₆-alkanedienediyl group which forms a fused-on ring, and, if
   G preferably represents one of the groups 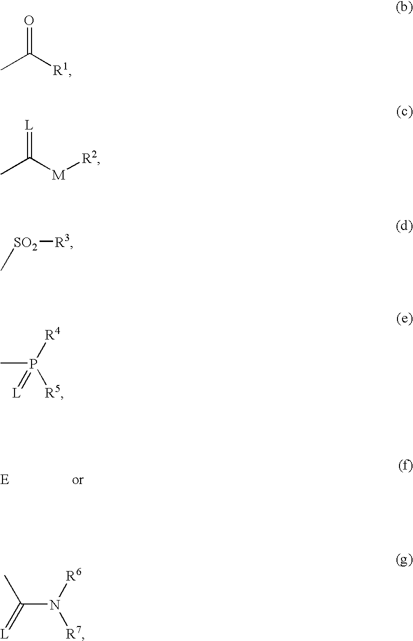
   in which
   E represents a metal ion equivalent or an ammonium ion,
   L represents oxygen or sulphur and
   M represents oxygen or sulphur,
R¹ preferably represents C₁-C₂₀-alkyl, C₂-C₂₀-alkenyl, C₁-C₆-alkoxy-C₁-C₆-alkyl, C₁-C₆-alkylthio-C₁-C₆-alkyl or poly-C₁-C₄-alkoxy-C₁-C₄-alkyl, each of which is optionally mono- to heptasubstituted by halogen, mono- or disubstituted by cyano, monosubstituted by COR¹³, C═N—OR¹³, CO₂R¹³ or 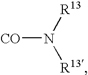
or represents C₃-C₈-cycloalkyl which is optionally mono- to trisubstituted by halogen, C₁-C₄-alkyl or C₁-C₄-alkoxy and in which optionally one or two not directly adjacent methylene groups are replaced by oxygen and/or sulphur,
   represents phenyl, phenyl-C₁-C₂-alkyl or phenyl-C₂-alkenyl, each of which is optionally mono- to trisubstituted by halogen, cyano, nitro, C₁-C₆-alkyl, C₁-C₆-alkoxy, C₁-C₆-haloalkyl, C₁-C₆-haloalkoxy, C₁-C₆-alkylthio, C₁-C₆-alkylsulfinyl or C₁-C₆-alkylsulfonyl,
   represents 5- or 6-membered heteroaryl which is optionally mono- or disubstituted by halogen or C₁-C₆-alkyl and contains one or two heteroatoms from the group consisting of oxygen, sulphur and nitrogen,
R² preferably represents C₁-C₂₀-alkyl, C₂-C₂₀-alkenyl, C₁-C₆-alkoxy-C₂-C₆-alkyl or poly-C₁-C₆-alkoxy-C₂-C₆-alkyl, each of which is optionally mono- to trisubstituted by halogen,
   represents C₃-C₈-cycloalkyl which is optionally mono- or disubstituted by halogen, C₁-C₆-alkyl or C₁-C₆-alkoxy or
   represents phenyl or benzyl, each of which is optionally mono- to trisubstituted by halogen, cyano, nitro, C₁-C₆-alkyl, C₁-C₆-alkoxy, C₁-C₆-haloalkyl or C₁-C₆-haloalkoxy,
R³ preferably represents C₁-C₈-alkyl which is optionally mono- or polysubstituted by halogen or represents phenyl or benzyl, each of which is optionally mono- or disubstituted by halogen, C₁-C₆-alkyl, C₁-C₆-alkoxy, C₁-C₄-haloalkyl, C₁-C₄-haloalkoxy, cyano or nitro,
R⁴ and R⁵ independently of one another preferably represent C₁-C₈-alkyl, C₁-C₈-alkoxy, C₁-C₈-alkylamino, di-(C₁-C₈-alkyl)amino, C₁-C₈-alkylthio or C₂-C₈-alkenylthio, each of which is optionally mono- to trisubstituted by halogen, or represent phenyl, phenoxy or phenylthio, each of which is optionally mono- to trisubstituted by halogen, nitro, cyano, C₁-C₄-alkoxy, C₁-C₄-haloalkoxy, C₁-C₄-alkylthio, C₁-C₄-haloalkylthio, C₁-C₄-alkyl or C₁-C₄-haloalkyl,
R⁶ and R⁷ independently of one another preferably represent hydrogen, represent C₁-C₈-alkyl, C₃-C₈-cycloalkyl, C₁-C₈-alkoxy, C₃-C₈-alkenyl or C₁-C₈-alkoxy-C₂-C₈-alkyl, each of which is optionally mono- to trisubstituted by halogen, represent phenyl or benzyl, each of which is optionally mono- to trisubstituted by halogen, C₁-C₈-alkyl, C₁-C₈-haloalkyl or C₁-C₈-alkoxy or together represent a C₃-C₆-alkylene radical which is optionally mono- or disubstituted by C₁-C₄-alkyl and in which optionally one methylene group is replaced by oxygen or sulphur,
R¹³ preferably represents C₁-C₆-alkyl, C₃-C₆-alkenyl, C₃-C₆-alkynyl or C₁-C₄-alkoxy-C₂-C₄-alkyl, each of which is optionally mono- to trisubstituted by halogen, or represents C₃-C₆-cycloalkyl which is optionally mono- or disubstituted by halogen, C₁-C₂-alkyl or C₁-C₂-alkoxy and in which optionally one or two not directly adjacent methylene groups are replaced oxygen,
R^{13′} preferably represents hydrogen, C₁-C₆-alkyl or C₃-C₆-alkenyl, then
A preferably represents hydrogen, represents C₁-C₈-alkyl, C₂-C₈-alkenyl, C₁-C₆-alkoxy-C₁-C₄-alkyl or C₁-C₆-alkylthio-C₁-C₄-alkyl, each of which is optionally mono- to trisubstituted by halogen, represents C₃-C₈-Cycloalkyl which is optionally mono- to trisubstituted by halogen, C₁-C₆-alkyl or C₁-C₆-alkoxy,
B preferably represents hydrogen, C₁-C₆-alkyl or C₁-C₄-alkoxy-C₁-C₂-alkyl,
D preferably represents hydrogen, represents C₁-C₈-alkyl, C₁-C₈-alkenyl, C₁-C₆-alkoxy-C₂-C₄-alkyl or C₁-C₆-alkylthio-C₂-C₄-alkyl, each of which is optionally mono- to trisubstituted by halogen, represents C₃-C₈-cycloalkyl which is optionally mono- to trisubstituted by halogen, C₁-C₄-alkyl, C₁-C₄-alkoxy or C₁-C₂-haloalkyl, or
A and D together preferably represent a C₃-C₆-alkanediyl or C₃-C₆-alkenediyl group in which in each case optionally one methylene group is replaced by oxygen or sulphur and which are in each case optionally mono- or disubstituted by halogen, hydroxyl, C₁-C₄-alkyl or C₁-C₄-alkoxy or by a further C₃-C₆-alkanediyl, C₃-C₆-alkenediyl or C₄-C₆-alkanedienediyl group which forms a fused-on ring.

In the radical definitions mentioned as being preferred, halogen represents fluorine, chlorine, bromine and iodine, in particular fluorine, chlorine and bromine.

If
G particularly preferably represents hydrogen (a), then
A particularly preferably represents hydrogen or C₁-C₆-alkyl,
B particularly preferable represents hydrogen or C₁-C₄-alkyl,
D particularly preferably represents C₁-C₆-alkyl, C₃-C₆-alkenyl, C₁-C₄-alkoxy-C₂-C₃-alkyl or C₁-C₄-alkylthio-C₂-C₃-alkyl, each of which is optionally mono- to trisubstituted by fluorine or chlorine, represents C₃-C₆-cycloalkyl which is optionally mono- or disubstituted by fluorine, chlorine, C₁-C₂-alkyl, C₁-C₂-alkoxy or trifluoromethyl, or
A and D together particularly preferably represent a C₃-C₅-alkanediyl group in which optionally one methylene group is replaced oxygen or sulphur and which is optionally mono- or disubstituted by C₁-C₂-alkyl or C₁-C₂-alkoxy,
or A and D together with the atoms to which they are attached represent one of the groups AD-1 to AD-10 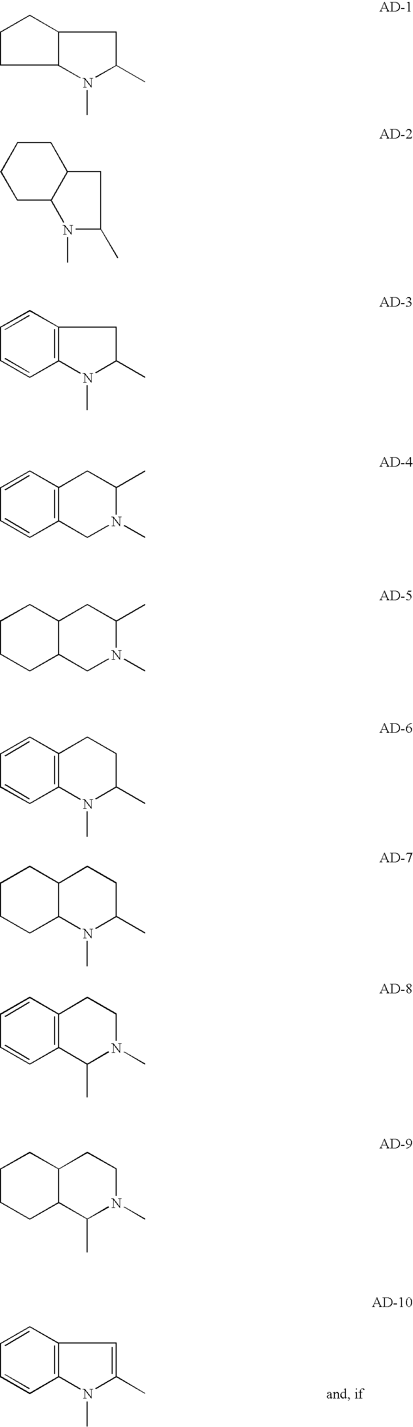
G particularly preferably represents one of the groups 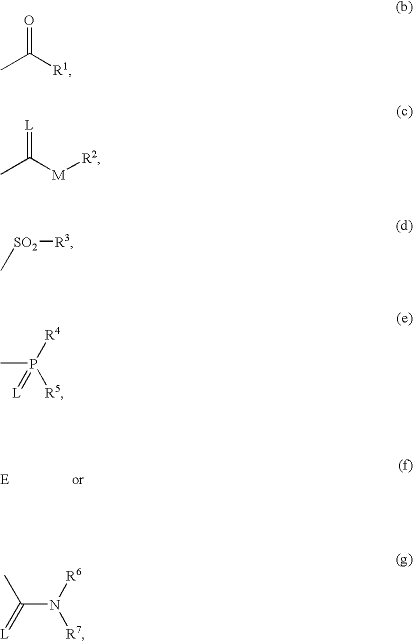
in which
   E represents a metal ion equivalent or an ammonium ion,
   L represents oxygen or sulphur and
   M represents oxygen or sulphur,
R¹ particularly preferably represents C₁-C₁₀-alkyl, C₂-C₁₀-alkenyl, C₁-C₄-alkoxy-C₁-C₂-alkyl, poly-C₁-C₃-alkoxy-C₁-C₂-alkyl or C₁-C₄-alkylthio-C₁-C₂-alkyl, each of which is optionally mono- to pentasubstituted by fluorine or chlorine, monosubstituted by cyano or monosubstituted by CO—R¹³, C═N—OR¹³ or CO₂R¹³, or represents C₃-C₆-cycloalkyl which is optionally mono- or disubstituted by fluorine, chlorine, C₁-C₂-alkyl or C₁-C₂-alkoxy and in which optionally one or two not directly adjacent methylene groups are replaced by oxygen,
   represents phenyl or benzyl, each of which is optionally mono- or disubstituted by fluorine, chlorine, bromine, cyano, nitro, C₁-C₄-alkyl, C₁-C₄-alkylthio, C₁-C₄-alkylsulfinyl, C₁-C₄-alkylsulfonyl, C₁-C₄-alkoxy, C₁-C₂-haloalkyl or C₁-C₂-haloalkoxy,
   represents pyrazolyl, thiazolyl, pyridyl, pyrimidyl, furanyl or thienyl, each of which is optionally mono- or disubstituted by fluorine, chlorine, bromine or C₁-C₂-alkyl,
R² particularly preferably represents C₁-C₁₀-alkyl, C₂-C₁₀-alkenyl, C₁-C₄-alkoxy-C₂-C₄-alkyl or poly-C₁-C₄-alkoxy-C₂-C₄-alkyl, each of which is optionally mono- to trisubstituted by fluorine or chlorine,
   represents C₃-C₇-cycloalkyl which is optionally monosubstituted by C₁-C₂-alkyl or C₁-C₂-alkoxy or
   represents phenyl or benzyl, each of which is optionally mono- or disubstituted by fluorine, chlorine, bromine, cyano, nitro, C₁-C₄-alkyl, methoxy, trifluoromethyl or trifluoromethoxy,
R³ particularly preferably represents C₁-C₄-alkyl which is optionally mono- to trisubstituted by fluorine or chlorine or represents phenyl or benzyl, each of which is optionally monosubstituted by fluorine, chlorine, bromine, C₁-C₄-alkyl, C₁-C₄-alkoxy, trifluoromethyl, trifluoromethoxy, cyano or nitro,
R⁴ and R⁵ independently of one another particularly preferably represent C₁-C₆-alkyl, C₁-C₆-alkoxy, C₁-C₆-alkylamino, di(C₁-C₆-alkyl)amino, C₁-C₆-alkylthio or C₃-C₄-alkenylthio, each of which is optionally mono- to trisubstituted by fluorine or chlorine, or represent phenyl, phenoxy or phenylthio, each of which is optionally mono- or disubstituted by fluorine, chlorine, bromine, nitro, cyano, C₁-C₃-alkoxy, trifluoromethoxy, C₁-C₃-alkylthio, C₁-C₃-alkyl or trifluoromethyl,
R⁶ and R⁷ independently of one another particularly preferably represent hydrogen, represent C₁-C₆-alkyl, C₃-C₆-Cycloalkyl, C₁-C₄-alkoxy, C₃-C₆-alkenyl or C₁-C₆-alkoxy-C₂-C₆-alkyl, each of which is optionally mono- to trisubstituted by fluorine or chlorine, represent phenyl which is optionally mono- or disubstituted by fluorine, chlorine, bromine, trifluoromethyl, C₁-C₄-alkyl or C₁-C₄-alkoxy or together represent a C₅-C₆-alkylene radical which is optionally mono- or disubstituted by methyl and in which optionally one methylene group is replaced oxygen,
R¹³ represents C₁-C₄-alkyl, C₃-C₄-alkenyl, C₃-C₄-alkynyl or C₁-C₄-alkoxy-C₂-C₃-alkyl or represents C₃-C₆-cycloalkyl in which optionally one methylene group is replaced by oxygen, then
A particularly preferably represents hydrogen, represents C₁-C₆-alkyl, C₂-C₆-alkenyl, C₁-C₄-alkoxy-C₁-C₃-alkyl or C₁-C₄-alkylthio-C₁-C₃-alkyl, each of which is optionally mono- to trisubstituted by fluorine or chlorine, or represents C₃-C₆-cycloalkyl which is optionally mono- or disubstituted by fluorine, chlorine, C₁-C₂-alkyl or C₁-C₂-alkoxy,
B particularly preferably represents hydrogen, C₁-C₄-alkyl or C₁-C₄-alkoxy-C₁-C₂-alkyl,
D particularly preferably represents hydrogen or
D particularly preferably also represents C₁-C₆-alkyl, C³-C₆-alkenyl, C₁-C₄-alkoxy-C₂-C₃-alkyl or C₁-C₄-alkylthio-C₂-C₃-alkyl, each of which is optionally mono- to trisubstituted by fluorine or chlorine, represents C₃-C₆-cycloalkyl which is optionally mono- or disubstituted by fluorine, chlorine, C₁-C₂-alkyl, C₁-C₂-alkoxy or trifluoromethyl, with the proviso that in this case
   A only represents hydrogen or C₁-C₃-alkyl, or
A and D together particularly preferably represent a C₃-C₅-alkanediyl group in which optionally one methylene group is replaced by oxygen or sulphur and which is optionally mono- or disubstituted by C₁-C₂-alkyl or C₁-C₂-alkoxy,
or A and D together with the atoms to which they are attached represent one of the groups AD-1 to AD-10

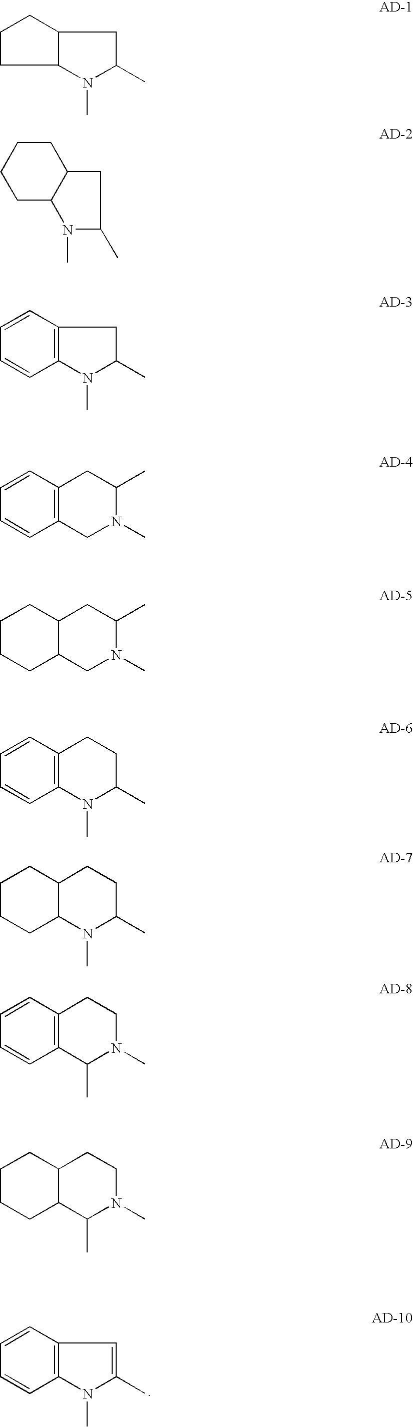

In the radical definitions mentioned as being particularly preferred, halogen represents fluorine, chlorine and bromine, in particular fluorine and chlorine.

If
G very particularly preferably represents hydrogen (a), then
A very particularly preferably represents hydrogen, methyl or ethyl,
B very particularly preferably represents hydrogen,
D very particularly preferably represents methyl, ethyl, n-propyl, isopropyl, n-butyl, sec-butyl, isobutyl, cyclopropyl, cyclopentyl or cyclohexyl,
A and D together very particularly preferably represent a C₃-C₄-alkanediyl group in which optionally one methylene group is replaced by oxygen or sulphur and which is optionally mono- or disubstituted by methyl,
   or A and D together with the atoms to which they are attached represent the following group: 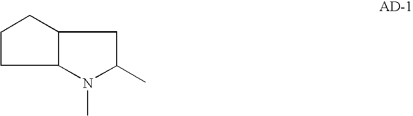
   and, if
G very particularly preferably represents one of the groups 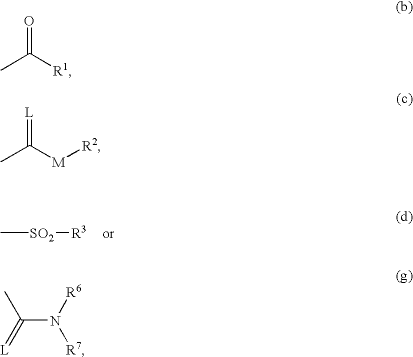
in which
   L represents oxygen and
   M represents oxygen or sulphur,
R¹ very particularly preferably represents C₁-C₆-alkyl, C₂-C₆-alkenyl, C₁-C₂-alkoxy-C₁-C₂-alkyl, C₁-C₂-alkylthio-C₁-C₂-alkyl or poly-C₁-C₂-alkoxy-C₁-C₂-alkyl, each of which is optionally mono- to trisubstituted by fluorine or chlorine, or represents cyclopropyl, cyclopentyl or cyclohexyl, each of which is optionally monosubstituted by fluorine, chlorine, methyl, ethyl or methoxy,
   represents phenyl which is optionally monosubstituted by fluorine, chlorine, bromine, cyano, nitro, methyl, ethyl, n-propyl, isopropyl, methoxy, ethoxy, methylthio, ethylthio, methylsulfinyl, ethylsulfinyl, methylsulfonyl, ethylsulfonyl, trifluoromethyl or trifluoromethoxy,
   represents furanyl, thienyl or pyridyl, each of which is optionally monosubstituted by chlorine, bromine or methyl,
R² very particularly preferably represents C₁-C₈-alkyl, C₂-C₆-alkenyl or C₁-C₃-alkoxy-C₂-C₃-alkyl, cyclopentyl or cyclohexyl,
   or represents phenyl or benzyl, each of which is optionally monosubstituted by fluorine, chlorine, bromine, cyano, nitro, methyl, methoxy, trifluoromethyl or trifluoromethoxy,
R³ very particularly preferably represents C₁-C₄-alkyl which is optionally mono- to trisubstituted by fluorine or chlorine, or represents phenyl or benzyl, each of which is optionally monosubstituted by fluorine, chlorine, bromine, C₁-C₄-alkyl, C₁-C₄-alkoxy, trifluoromethyl, trifluoromethoxy, cyano or nitro,
R⁶ very particularly preferably represents hydrogen, represents C₁-C₄-alkyl, C₃-C₆-cycloalkyl or allyl, represents phenyl which is optionally monosubstituted by fluorine, chlorine, bromine, methyl, methoxy or trifluoromethyl,
R⁷ very particularly preferably represents methyl, ethyl, n-propyl, isopropyl or allyl,
R⁶ and R⁷ together very particularly preferably represent a C₅-C₆-alkylene radical in which optionally one methylene group is replaced by oxygen, then
A very particularly preferably represents hydrogen, methyl, ethyl, n-propyl, isopropyl, n-butyl, isobutyl, sec-butyl, tert-butyl, trifluoromethyl, cyclopropyl, cyclopentyl or cyclohexyl,
B very particularly preferably represents hydrogen, methyl or ethyl,
D very particularly preferably represents hydrogen or
D very particularly preferably also represents methyl, ethyl, n-propyl, isopropyl, n-butyl, sec-butyl, isobutyl, cyclopropyl, cyclopentyl or cyclohexyl, with the proviso that in this case
A only represents hydrogen, methyl or ethyl,
A and D together very particularly preferably represent a C₃-C₄-alkanediyl group in which optionally one methylene group is replaced by oxygen or sulphur and which is optionally mono- or disubstituted by methyl, or
A and D together with the atoms to which they are attached represent the group below:

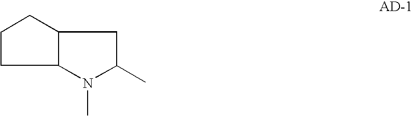

If
G especially represents hydrogen (a), then
A especially represents hydrogen, methyl or ethyl,
B especially represents hydrogen,
D especially represents methyl, ethyl or cyclopropyl, cyclopentyl,
A and D together with the atoms to which they are attached especially represent the group below: 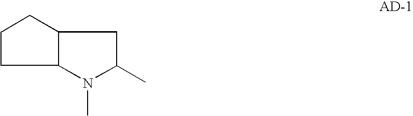
and, if
G especially represents one of the groups 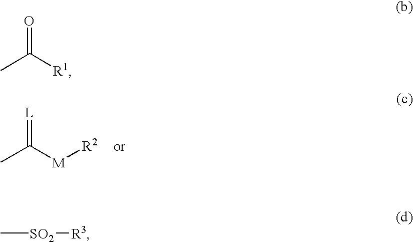
in which
   L represents oxygen and
   M represents oxygen,
R¹ especially represents C₁-C₆-alkyl or C₁-C₂-alkoxy-C₁-C₂-alkyl,
R² especially represents C₁-C₈-alkyl,
R³ especially represents C₁-C₄-alkyl, then
A especially represents hydrogen, methyl, ethyl, n-propyl, isopropyl or isobutyl,
B especially represents hydrogen, methyl or ethyl,
D especially represents hydrogen or
D also especially represents methyl, ethyl or cyclopropyl, with the proviso that in this case
A only represents hydrogen, methyl or ethyl,
A and D together with the atoms to which they are attached especially represent the group below:

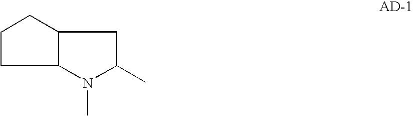

The general or preferred radical definitions or illustrations listed above can be combined with one another as desired, i.e. including combinations between the respective ranges and preferred ranges. They apply to the end products and, correspondingly, to the precursors and intermediates.

Preference according to the invention is given to the compounds of the formula (I) which contain a combination of the meanings listed above as being preferred (preferable).

Particular preference according to the invention is given to the compounds of the formula (I) which contain a combination of the meanings listed above as being particularly preferred.

Very particular preference according to the invention is given to the compounds of the formula (I) which contain a combination of the meanings listed above as being very particularly preferred.

Especially preferred according to the invention are the compounds of the formula (I) which contain a combination of the meanings listed above as being especially preferred.

Saturated or unsaturated hydrocarbon radicals, such as alkyl, alkanediyl or alkenyl, can in each case be straight-chain or branched as far as this is possible, including in combination with heteroatoms, such as, for example, in alkoxy.

Optionally substituted radicals can be mono- or polysubstituted, where in the case of polysubstitution the substituents can be identical or different.

In addition to the compounds mentioned in the Preparation Examples, the following compounds of the formulae (I-b) and (I-c) may be specifically mentioned:

TABLE 1

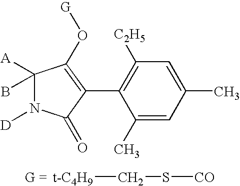

| A | B | D |
|---|---|---|
| CH₃ | H | H |
| C₂H₅ | H | H |
| C₃H₇ | H | H |
| i-C₃H₇ | H | H |
| C₄H₉ | H | H |
| i-C₄H₉ | H | H |
| s-C₄H₉ | H | H |
| t-C₄H₉ | H | H |
| CH₃ | CH₃ | H |
| C₂H₅ | CH₃ | H |
| C₃H₇ | CH₃ | H |
| i-C₃H₇ | CH₃ | H |
| C₄H₉ | CH₃ | H |
| i-C₄H₉ | CH₃ | H |
| s-C₄H₉ | CH₃ | H |
| t-C₄H₉ | CH₃ | H |
| C₂H₅ | C₂H₅ | H |
| 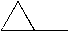 | CH₃ | H |
| 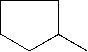 | CH₃ | H |
| 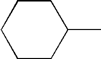 | CH₃ | H |
| A | D | B |
| —(CH₂)₃— | | H |
| —(CH₂)₄— | | H |
| —CH₂—CHCH₃—CH₂— | | H |
| —CH₂—CH₂—CHCH₃— | | H |
| —CH₂—CHCH₃—CHCH₃— | | H |
| —CH₂—S—(CH₂)₂— | | H |
| 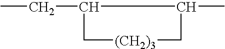 | | H |
| H | CH₃ | H |
| H | C₂H₅ | H |
| H | C₃H₇ | H |
| H | i-C₃H₇ | H |
| H | 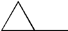 | H |
| H | 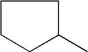 | H |
| H | 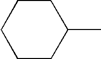 | H |
| CH₃ | CH₃ | H |
| CH₃ | C₂H₅ | |
| CH₃ | C₃H₇ | |
| CH₃ | i-C₃H₇ | |
| CH₃ | 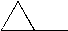 | H |
| CH₃ | 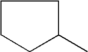 | H |
| CH₃ | 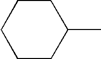 | H |
| C₂H₅ | CH₃ | H |
| C₂H₅ | C₂H₅ | H |

A, B and D as in Table 1

| | |
|---|---|
| Table 2 | G = CH₃—CO |
| Table 3 | G = C₂H₅—CO |
| Table 4 | G = C₃H₇—CO |
| Table 5 | G = i-C₃H₇—CO |
| Table 6 | G = C₄H₉—CO |
| Table 7 | G = i-C₄H₉—CO |
| Table 8 | G = s-C₄H₉—CO |
| Table 9 | G = t-C₄H₉—CO |
| Table 10 | 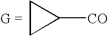 |
| Table 11 | G = H₃C—O—CH₂—CO |
| Table 12 | G = H₅C₂—O—CH₂—CO |
| Table 13 | G = H₃C—S—CH₂—CO |
| Table 14 | G = H₅C₂—S—CH₂—CO |
| Table 15 | G = CH₃—O—CO |
| Table 16 | G = C₂H₅—O—CO |
| Table 17 | G = C₃H₇—O—CO |
| Table 18 | G = i-C₃H₇—O—CO |
| Table 19 | G = C₄H₉—O—CO |
| Table 20 | G = i-C₄H₉—O—CO |
| Table 21 | G = s-C₄H₉—O—CO |
| Table 22 | G = t-C₄H₉—O—CO |
| Table 23 | G = t-C₄H₉—CH₂—O—CO |
| Table 24 | G = C₆H₅—CH₂—O—CO |
| Table 25 | G = C₆H₅—O—CO |
| Table 26 | G = CH₃—S—CO |
| Table 27 | G = C₂H₅—S—CO |
| Table 28 | G = C₃H₇—S—CO |
| Table 29 | G = i-C₃H₇—S—CO |
| Table 30 | G = C₄H₉—S—CO |
| Table 31 | G = i-C₄H₉—S—CO |
| Table 32 | G = s-C₄H₉—S—CO |
| Table 33 | G = t-C₄H₉—S—CO |
| Table 34 | G = C₆H₅—CH₂—S—CO |
| Table 35 | G = C₆H₅—S—CO |

In addition to the compounds mentioned in the Preparation Examples, the following compounds of the formula (I-a) may also be specifically mentioned:

| | |
|---|---|
| | (I-a) |
| 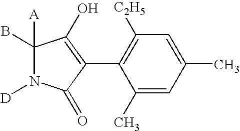 | |

| | | |
|---|---|---|
| A | D | B |

| —(CH₂)₃— | | H |
|---|---|---|
| —(CH₂)₄— | | H |
| —CH₂—CHCH₃—CH₂— | | H |
| —CH₂—CH₂—CHCH₃— | | H |
| —CH₂—CHCH₃—CHCH₃— | | H |
| —CH₂—S—(CH₂)₂— | | H |
| 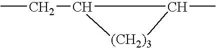 | | H |
| H | CH₃ | H |
| H | C₂H₅ | H |
| H | C₃H₇ | H |
| H | i-C₃H₇ | H |
| H | 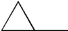 | H |
| H | 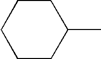 | H |
| CH₃ | CH₃ | H |
| CH₃ | C₂H₅ | |
| CH₃ | C₃H₇ | |
| CH₃ | i-C₃H₇ | |
| CH₃ | 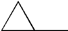 | H |
| CH₃ | 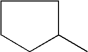 | H |
| CH₃ | 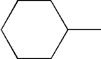 | H |
| C₂H₅ | CH₃ | H |
| C₂H₅ | C₂H₅ | H |

Preferred meanings of the cyclic ketoenols of the formula (I-a′) mentioned above as active compounds in combination with the crop plant compatibility-improving compounds (herbicide safeners) are defined below.

Preference is given to compounds of the formula (I-a′), in which
G preferably represents hydrogen (a),
A preferably represents hydrogen, C₁-C₈-alkyl, C₁-C₄-haloalkyl or represents C₃-C₈-alkyl which is optionally mono- to trisubstituted by halogen, C₁-C₆-alkyl or C₁-C₆-alkoxy or represents C₃-C₆-cycloalkyl which is optionally mono- to trisubstituted by halogen, C₁-C₄-alkyl or C₁-C₄-alkoxy,
B preferably represents hydrogen, C₁-C₆-alkyl or C₁-C₆-alkoxy-C₁-C₄-alkyl.

Particular preference is given to compounds of the formula (I-a′), in which
G particularly preferably represents hydrogen (a),
A particularly preferably represents hydrogen, C₁-C₆-alkyl, C₁-C₂-haloalkyl having one to three fluorine atoms or C₁-C₄-alkoxy-C₁-C₃-alkyl or represents C₃-C₆-cycloalkyl which is optionally mono- or disubstituted by fluorine, chlorine, C₁-C₂-alkyl or C₁-C₂-alkoxy,
B particularly preferably represents hydrogen, C₁-C₂-alkyl or C₁-C₄-alkoxy-C₁-C₂-alkyl.

Very particular preference is given to compounds of the formula (I-a′) in which
G very particularly preferably represents hydrogen (a),
A very particularly preferably represents methyl, ethyl, n-propyl, isopropyl, n-butyl, isobutyl, sec-butyl, tert-butyl, trifluoromethyl, cyclopropyl, cyclopentyl or cyclohexyl,
B very particularly preferably represents hydrogen, methyl or ethyl.

Especially preferred are the compounds of the formula (I-a′) mentioned in the table below:

TABLE

| | |
|---|---|
| | (I-a′) |
| 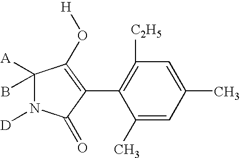 | |

| A | B | D |
|---|---|---|
| CH₃ | H | H |
| C₂H₅ | H | H |
| C₃H₇ | H | H |
| i-C₃H₇ | H | H |
| C₄H₉ | H | H |
| i-C₄H₉ | H | H |
| s-C₄H₉ | H | H |
| t-C₄H₉ | H | H |
| CH₃ | CH₃ | H |
| C₂H₅ | CH₃ | H |
| C₃H₇ | CH₃ | H |
| i-C₃H₇ | CH₃ | H |
| C₄H₉ | CH₃ | H |
| i-C₄H₉ | CH₃ | H |
| s-C₄H₉ | CH₃ | H |
| t-C₄H₉ | CH₃ | H |
| C₂H₅ | C₂H₅ | H |
| 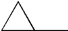 | CH₃ | H |
| 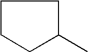 | CH₃ | H |
| 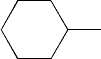 | CH₃ | H |

Preferred meanings of the groups listed above in connection with the crop plant compatibility-improving compounds (“herbicide safeners”) of the formulae (IIa), (IIb), (IIc), (IId) and (IIe) are defined below.
m preferably represents the number 0, 1, 2, 3 or 4,
A¹ preferably represents one of the divalent heterocyclic groupings shown below 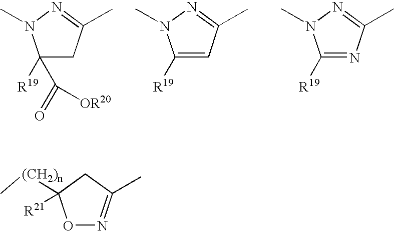
n preferably represents the number 0, 1, 2, 3 or 4.
A² preferably represents in each case optionally methyl-, ethyl-, methoxycarbonyl-, ethoxycarbonyl- or allyloxycarbonyl-substituted methylene or ethylene,
R¹⁴ preferably represents hydroxyl, mercapto, amino, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino or diethylamino,
R¹⁵ preferably represents hydroxyl, mercapto, amino, methoxy, ethoxy, n- or i-propoxy, n-, i-, s- or t-butoxy, 1-methylhexyloxy, allyloxy, 1-allyloxymethylethoxy, methylthio, ethylthio, n- or i-propylthio, n-, i-, s- or t-butylthio, methylamino, ethylamino, n- or i-propylamino, n-, i-, s- or t-butylamino, dimethylamino or diethylamino,
R¹⁶ preferably represents in each case optionally fluorine-, chlorine- and/or bromine-substituted methyl, ethyl, n- or i-propyl,
R¹⁷ preferably represents hydrogen, in each case optionally fluorine- and/or chlorine-substituted methyl, ethyl, n- or i-propyl, n-, i-, s- or t-butyl, propenyl, butenyl, propynyl or butynyl, methoxymethyl, ethoxymethyl, methoxyethyl, ethoxyethyl, dioxolanylmethyl, furyl, furylmethyl, thienyl, thiazolyl, piperidinyl, or optionally fluorine-, chlorine-, methyl-, ethyl-, n- or i-propyl-, n-, i-, s- or t-butyl-substituted phenyl,
R¹⁸ preferably represents hydrogen, in each case optionally fluorine- and/or chlorine-substituted methyl, ethyl, n- or i-propyl, n-, i-, s- or t-butyl, propenyl, butenyl, propynyl or butynyl, methoxymethyl, ethoxymethyl, methoxyethyl, ethoxyethyl, dioxolanylmethyl, furyl, furylmethyl, thienyl, thiazolyl, piperidinyl, or optionally fluorine-, chlorine-, methyl-, ethyl-, n- or i-propyl-, n-, i-, s- or t-butyl-substituted phenyl, or together with R¹⁷ represents one of the radicals —CH₂—O—CH₂—CH₂— and —CH₂—CH₂—O—CH₂—CH₂— which are optionally substituted by methyl, ethyl, furyl, phenyl, a fused benzene ring or by two substituents which, together with the C atom to which they are attached, form a 5- or 6-membered carbocycle,
R¹⁹ preferably represents hydrogen, cyano, fluorine, chlorine, bromine, or represents in each case optionally, fluorine-, chlorine- and/or bromine-substituted methyl, ethyl, n- or i-propyl, cyclopropyl, cyclobutyl, cyclopentyl, cyclohexyl or phenyl,
R²⁰ preferably represents hydrogen, optionally hydroxyl-, cyano-, fluorine-, chlorine-, methoxy-, ethoxy-, n- or i-propoxy-substituted methyl, ethyl, n- or i-propyl, n-, i-, s- or t-butyl,
R²¹ preferably represents hydrogen, cyano, fluorine, chlorine, bromine, or represents in each case optionally fluorine-, chlorine- and/or bromine-substituted methyl, ethyl, n- or i-propyl, n-, i-, s- or t-butyl, cyclopropyl, cyclobutyl, cyclopentyl, cyclohexyl or phenyl,
X¹ preferably represents nitro, cyano, fluorine, chlorine, bromine, methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, n- or i-propoxy, difluoromethoxy or trifluoromethoxy,
X² preferably represents hydrogen, nitro, cyano, fluorine, chlorine, bromine, methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, n- or i-propoxy, difluoromethoxy or trifluoromethoxy,
X³ preferably represents hydrogen, nitro, cyano, fluorine, chlorine, bromine, methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, n- or i-propoxy, difluoromethoxy or trifluoromethoxy,
t preferably represents the number 0, 1, 2, 3 or 4,
v preferably represents the number 0, 1, 2, 3 or 4,
R²² preferably represents hydrogen, methyl, ethyl, n- or i-propyl,
R²³ preferably represents hydrogen, methyl, ethyl, n- or i-propyl,
R²⁴ preferably represents hydrogen, in each case optionally cyano-, fluorine-, chlorine-, methoxy-, ethoxy-, n- or i-propoxy-substituted methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylamino, ethylamino, n- or i-propylamino, n-, i-, s- or t-butylamino, dimethylamino or diethylamino, or in each case optionally cyano-, fluorine-, chlorine-, bromine-, methyl-, ethyl-, n- or i-propyl-substituted cyclopropyl, cyclobutyl, cyclopentyl, cyclohexyl, cyclopropyloxy, cyclobutyloxy, cyclopentyloxy, cyclohexyloxy, cyclopropylthio, cyclobutylthio, cyclopentylthio, cyclohexylthio, cyclopropylamino, cyclobutylamino, cyclopentylamino or cyclohexylamino,
R²⁵ preferably represents hydrogen, in each case optionally cyano-, hydroxyl-, fluorine-, chlorine-, methoxy-, ethoxy-, n- or i-propoxy-substituted methyl, ethyl, n- or i-propyl, n-, i- or s-butyl, in each case optionally cyano-, fluorine-, chlorine- or bromine-substituted propenyl, butenyl, propynyl or butynyl, or in each case optionally cyano-, fluorine-, chlorine-, bromine-, methyl-, ethyl-, n- or i-propyl-substituted cyclopropyl, cyclobutyl, cyclopentyl or cyclohexyl,
R²⁶ preferably represents hydrogen, in each case optionally cyano-, hydroxyl-, fluorine-, chlorine-, methoxy-, ethoxy-, n- or i-propoxy-substituted methyl, ethyl, n- or i-propyl, n-, i- or s-butyl, in each case optionally cyano-, fluorine-, chlorine- or bromine-substituted propenyl, butenyl, propynyl or butynyl, in each case optionally cyano-, fluorine-, chlorine-, bromine-, methyl-, ethyl-, n- or i-propyl-substituted cyclopropyl, cyclobutyl, cyclopentyl or cyclohexyl, or optionally nitro-, cyano-, fluorine-, chlorine-, bromine-, methyl-, ethyl-, n- or i-propyl-, n-, i-, s- or t-butyl-, trifluoromethyl-, methoxy-, ethoxy-, n- or i-propoxy-, difluoromethoxy- or trifluoromethoxy-substituted phenyl, or together with R²⁵ represents in each case optionally methyl- or ethyl-substituted butane-1,4-diyl (trimethylene), pentane-1,5-yl, 1-oxabutane-1,4-diyl or 3-oxapentane-1,5-diyl,
X⁴ preferably represents nitro, cyano, carboxyl, carbamoyl, formyl, sulphamoyl, hydroxyl, amino, fluorine, chlorine, bromine, methyl, ethyl, n- or i-propyl, n-, i-, s- or t-butyl, trifluoromethyl, methoxy, ethoxy, n- or i-propoxy, difluoromethoxy or trifluoromethoxy,
X⁵ preferably represents nitro, cyano, carboxyl, carbamoyl, formyl, sulphamoyl, hydroxyl, amino, fluorine, chlorine, bromine, methyl, ethyl, n- or i-propyl, n-, i-, s- or t-butyl, trifluoromethyl, methoxy, ethoxy, n- or i-propoxy, difluoromethoxy or trifluoromethoxy.

Examples of the compounds of the formula (IIa) which are very particularly preferred as herbicide safeners according to the invention are listed in Table 2 below.

TABLE 2
Examples of the compounds of the formula (IIa)

| | |
|---|---|
| | (IIa) |
| 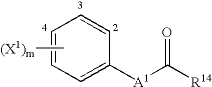 | |

| Example | (positions) | | |
|---|---|---|---|
| No. | (X¹)ₘ | A¹ | R¹⁴ |
| IIa-1 | (2) Cl, (4) Cl | 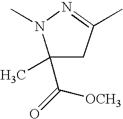 | OCH₃ |
| IIa-2 | (2) Cl, (4) Cl | 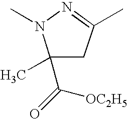 | OCH₃ |
| IIa-3 | (2) Cl, (4) Cl | 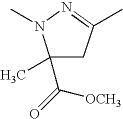 | OC₂H₅ |
| IIa-4 | (2) Cl, (4) Cl | 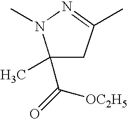 | OC₂H₅ |
| IIa-5 | (2) Cl | 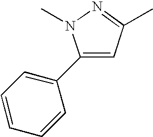 | OCH₃ |
| IIa-6 | (2) Cl, (4) Cl | 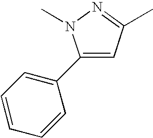 | OCH₃ |
| IIa-7 | (2) F | 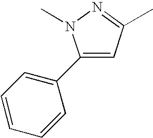 | OCH₃ |
| IIa-8 | (2) F | 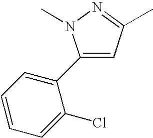 | OCH₃ |
| IIa-9 | (2) Cl, (4) Cl | 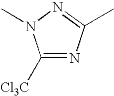 | OC₂H₅ |
| IIa-10 | (2) Cl, (4) CF₃ | 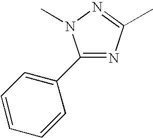 | OCH₃ |
| IIa-11 | (2) Cl | 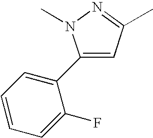 | OCH₃ |
| IIa-12 | — | 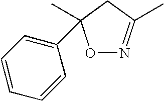 | OC₂H₅ |
| IIa-13 | (2) Cl, (4) Cl | 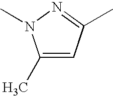 | OC₂H₅ |
| IIa-14 | (2) Cl, (4) Cl | 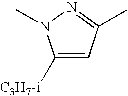 | OC₂H₅ |
| IIa-15 | (2) Cl, (4) Cl | 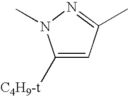 | OC₂H₅ |
| IIa-16 | (2) Cl, (4) Cl | 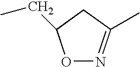 | OC₂H₅ |
| IIa-17 | (2) Cl, (4) Cl | 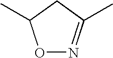 | OC₂H₅ |
| IIa-18 | — | 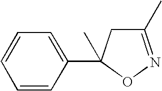 | OH |

Examples of the compounds of the formula (IIb) which are very particularly preferred as herbicide safeners according to the invention are listed in Table 3 below.

TABLE 3
Examples of the compounds of the formula (IIb)

| | |
|---|---|
| | (IIb) |
| 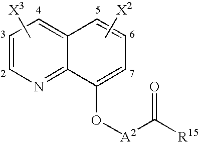 | |

| | (po- | | | |
|---|---|---|---|---|
| | si- | | | |
| Example | tion) | (position) | | |
| No. | X² | X³ | A² | R¹⁵ |
| IIb-1 | (5) | — | CH₂ | OH |
| | Cl | | | |
| IIb-2 | (5) | — | CH₂ | OCH₃ |
| | Cl | | | |
| IIb-3 | (5) | — | CH₂ | OC₂H₅ |
| | Cl | | | |
| IIb-4 | (5) | — | CH₂ | OC₃H₇-n |
| | Cl | | | |
| IIb-5 | (5) | — | CH₂ | OC₃H₇-i |
| | Cl | | | |
| IIb-6 | (5) | — | CH₂ | OC₄H₉-n |
| | Cl | | | |
| IIb-7 | (5) | — | CH₂ | OCH(CH₃)C₅H₁₁-n |
| | Cl | | | |
| IIb-8 | (5) | (2) | CH₂ | OH |
| | Cl | F | | |
| IIb-9 | (5) | (2) | CH₂ | OH |
| | Cl | Cl | | |
| IIb-10 | (5) | — | CH₂ | OCH₂CH═CH₂ |
| | Cl | | | |
| IIb-11 | (5) | — | CH₂ | OC₄H₉-i |
| | Cl | | | |
| IIb-12 | (5) Cl | — | CH₂ | 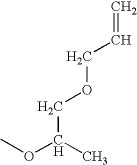 |
| IIb-13 | (5) Cl | — | 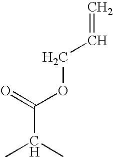 | OCH₂CH═CH₂ |
| IIb-14 | (5) Cl | — | 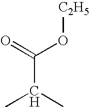 | OC₂H₅ |
| IIb-15 | (5) Cl | — | 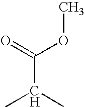 | OCH₃ |

Examples of the compounds of the formula (IIc) which are very particularly preferred as herbicide safeners according to the invention are listed in Table 4 below.

TABLE 4
Examples of the compounds of the formula (IIc)

| | |
|---|---|
| | (IIc) |
| 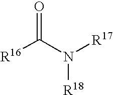 | |

| Example | | |
|---|---|---|
| No. | R¹⁶ | N(R¹⁷, R¹⁸) |
| IIc-1 | CHCl₂ | N(CH₂CH═CH₂)₂ |
| IIc-2 | CHCl₂ | 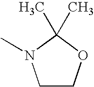 |
| IIc-3 | CHCl₂ | 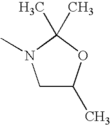 |
| IIc-4 | CHCl₂ | 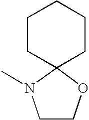 |
| IIc-5 | CHCl₂ | 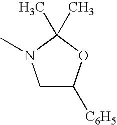 |
| IIc-6 | CHCl₂ | 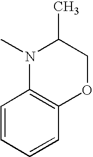 |
| IIc-7 | CHCl₂ | 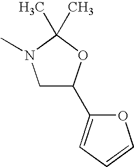 |

Examples of the compounds of the formula (IId) which are very particularly preferred as herbicide safeners according to the invention are listed in Table 5 below.

TABLE 5
Examples of the compounds of the formula (IId)

| | |
|---|---|
| | (IId) |
| 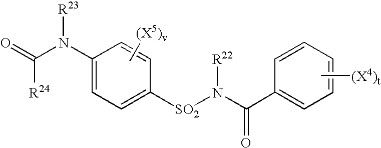 | |

| Example | | | | (positions) | (positions) |
|---|---|---|---|---|---|
| No. | R²² | R²³ | R²⁴ | (X⁴)ₜ | (X⁵)ᵥ |
| IId-1 | H | H | CH₃ | (2) OCH₃ | — |
| IId-2 | H | H | C₂H₅ | (2) OCH₃ | — |
| IId-3 | H | H | C₃H₇-n | (2) OCH₃ | — |
| IId-4 | H | H | C₃H₇-i | (2) OCH₃ | — |
| IId-5 | H | H | 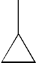 | (2) OCH₃ | — |
| IId-6 | H | H | CH₃ | (2) OCH₃ | — |
| | | | | (5) CH₃ | |
| IId-7 | H | H | C₂H₅ | (2) OCH₃ | — |
| | | | | (5) CH₃ | |
| IId-8 | H | H | C₃H₇-n | (2) OCH₃ | — |
| | | | | (5) CH₃ | |
| IId-9 | H | H | C₃H₇-i | (2) OCH₃ | — |
| | | | | (5) CH₃ | |
| IId-10 | H | H | 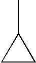 | (2) OCH₃(5) CH₃ | — |
| IId-11 | H | H | OCH₃ | (2) OCH₃ | — |
| | | | | (5) CH₃ | |
| IId-12 | H | H | OC₂H₅ | (2) OCH₃ | — |
| | | | | (5) CH₃ | |
| IId-13 | H | H | OC₃H₇-i | (2) OCH₃ | — |
| | | | | (5) CH₃ | |
| IId-14 | H | H | SCH₃ | (2) OCH₃ | — |
| | | | | (5) CH₃ | |
| IId-15 | H | H | SC₂H₅ | (2) OCH₃ | — |
| | | | | (5) CH₃ | |
| IId-16 | H | H | SC₃H₇-i | (2) OCH₃ | — |
| | | | | (5) CH₃ | |
| IId-17 | H | H | NHCH₃ | (2) OCH₃ | — |
| | | | | (5) CH₃ | |
| IId-18 | H | H | NHC₂H₅ | (2) OCH₃ | — |
| | | | | (5) CH₃ | |
| IId-19 | H | H | NHC₃H₇-i | (2) OCH₃ | — |
| | | | | (5) CH₃ | |
| IId-20 | H | H | 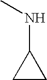 | (2) OCH₃(5) CH₃ | — |
| IId-21 | H | H | NHCH₃ | (2) OCH₃ | — |
| IId-22 | H | H | NHC₃H₇-i | (2) OCH₃ | — |
| IId-23 | H | H | N(CH₃)₂ | (2) OCH₃ | — |
| IId-24 | H | H | N(CH₃)₂ | (3) CH₃ | — |
| | | | | (4) CH₃ | |
| IId-25 | H | H | CH₂—O—CH₃ | (2) OCH₃ | — |

Examples of the compounds of the formula (IIe) which are very particularly preferred as herbicide safeners according to the invention are listed in Table 6 below.

TABLE 6
Examples of the compounds of the formula (IIe)

| | |
|---|---|
| | (IIe) |
| 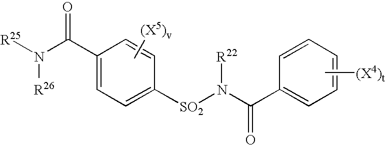 | |

| Example | | | | (positions) | (positions) |
|---|---|---|---|---|---|
| No. | R²² | R²⁵ | R²⁶ | (X⁴)ₜ | (X⁵)ᵥ |
| IIe-1 | H | H | CH₃ | (2) OCH₃ | — |
| IIe-2 | H | H | C₂H₅ | (2) OCH₃ | — |
| IIe-3 | H | H | C₃H₇-n | (2) OCH₃ | — |
| IIe-4 | H | H | C₃H₇-i | (2) OCH₃ | — |
| IIe-5 | H | H | 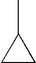 | (2) OCH₃ | — |
| IIe-6 | H | CH₃ | CH₃ | (2) OCH₃ | — |
| IIe-7 | H | H | CH₃ | (2) OCH₃ | — |
| | | | | (5) CH₃ | |
| IIe-8 | H | H | C₂H₅ | (2) OCH₃ | — |
| | | | | (5) CH₃ | |
| IIe-9 | H | H | C₃H₇-n | (2) OCH₃ | — |
| | | | | (5) CH₃ | |
| IIe-10 | H | H | C₃H₇-i | (2) OCH₃ | - — |
| | | | | (5) CH₃ | |
| IIe-11 | H | H | 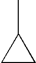 | (2) OCH₃(5) CH₃ | — |
| IIe-12 | H | CH₃ | CH₃ | (2) OCH₃ | — |
| | | | | (5) CH₃ | |

Most preferred as crop plant compatibility-improving compounds [component c′)] are cloquintocet-mexyl, fenchlorazole-ethyl, isoxadifen-ethyl, mefenpyr-diethyl, furilazole, fenclorim, cumyluron, dymron, dimepiperate and the compounds IIe-5 and IIe-11, and particular emphasis is given to cloquintocet-mexyl and mefenpyr-diethyl.

Examples of the selective herbicidal combinations according to the invention comprising in each case one active compound of the formula (I) and one of the safeners defined above are listed in Table 7 below.

TABLE 7
Examples of the combinations according to the invention

| Active compounds of the formula (I) | Safeners |
|---|---|
| I-a | cloquintocet-mexyl |
| I-a | fenchlorazole-ethyl |
| I-a | isoxadifen-ethyl |
| I-a | mefenpyr-diethyl |
| I-a | furilazole |
| I-a | fenclorim |
| I-a | cumyluron |
| I-a | daimuron/dymron |
| I-a | dimepiperate |
| I-a | IIe-11 |
| I-a | IIe-5 |
| I-b | cloquintocet-mexyl |
| I-b | fenchlorazole-ethyl |
| I-b | isoxadifen-ethyl |
| I-b | mefenpyr-diethyl |
| I-b | furilazole |
| I-b | fenclorim |
| I-b | cumyluron |
| I-b | daimuron/dymron |
| I-b | dimepiperate |
| I-b | IIe-11 |
| I-b | IIe-5 |
| I-c | cloquintocet-mexyl |
| I-c | fenchlorazole-ethyl |
| I-c | isoxadifen-ethyl |
| I-c | mefenpyr-diethyl |
| I-c | furilazole |
| I-c | fenclorim |
| I-c | cumyluron |
| I-c | daimuron/dymron |
| I-c | dimepiperate |
| I-c | IIe-5 |
| I-c | IIe-11 |
| I-d | cloquintocet-mexyl |
| I-d | fenchlorazole-ethyl |
| I-d | isoxadifen-ethyl |
| I-d | mefenpyr-diethyl |
| I-d | furilazole |
| I-d | fenclorim |
| I-d | cumyluron |
| I-d | daimuron/dymron |
| I-d | dimepiperate |
| I-d | IIe-11 |
| I-d | IIe-5 |
| I-e | cloquintocet-mexyl |
| I-e | fenchlorazole-ethyl |
| I-e | isoxadifen-ethyl |
| I-e | mefenpyr-diethyl |
| I-e | funlazole |
| I-e | fenclorim |
| I-e | cumyluron |
| I-e | daimuron/dymron |
| I-e | dimepiperate |
| I-e | IIe-5 |
| I-e | IIe-11 |
| I-f | cloquintocet-mexyl |
| I-f | fenchlorazole-ethyl |
| I-f | isoxadifen-ethyl |
| I-f | mefenpyr-diethyl |
| I-f | furilazole |
| I-f | fenclorim |
| I-f | cumyluron |
| I-f | daimuron/dymron |
| I-f | dimepiperate |
| I-f | IIe-5 |
| I-f | IIe-11 |
| I-g | cloquintocet-mexyl |
| I-g | fenchlorazole-ethyl |
| I-g | isoxadifen-ethyl |
| I-g | mefenpyr-diethyl |
| I-g | furilazole |
| I-g | fenclorim |
| I-g | cumyluron |
| I-g | daimuron/dymron |
| I-g | dimepiperate |
| I-g | IIe-5 |
| I-g | IIe-11 |
| I-a′ | cloquintocet-mexyl |
| I-a′ | fenchlorazole-ethyl |
| I-a′ | isoxadifen-ethyl |
| I-a′ | mefenpyr-diethyl |
| I-a′ | furilazole |
| I-a′ | fenclorim |
| I-a′ | cumyluron |
| I-a′ | daimuron/dymron |
| I-a′ | dimepiperate |
| I-a′ | II-5 |
| I-a′ | IIe-11 |

The compounds of the general formula (IIa) to be used as safeners according to the invention are known and/or can be prepared by processes known per se (cf. WO-A-91/07874, WO-A-95/07897).

The compounds of the general formula (IIb) to be used as safeners according to the invention are known and/or can be prepared by processes known per se (cf. EP-A-191736).

The compounds of the general formula (IIc) to be used as safeners according to the invention are known and/or can be prepared by processes known per se (cf. DE-A-2218097, DE-A-2350547).

The compounds of the general formula (IId) to be used as safeners according to the invention are known and/or can be prepared by processes known per se (cf. DE-A-19621522/U.S. Pat. No. 6,235,680).

The compounds of the general formula (IIe) to be used as safeners according to the invention are known and/or can be prepared by processes known per se (cf. WO-A-99/66795/U.S. Pat. No. 6,251,827).

Surprisingly, it has now been found that the active compound combinations defined above of substituted ketoenols of the general formulae (I) and (I-a′) and safeners (antidotes) from the component (c′) listed above, while having very good compatibility with useful plants, have a particularly high herbicidal activity and can be used in various crops, in particular in cereals (especially wheat), but also in soya beans, potatoes, maize and rice, for the selective control of weeds.

Here, it has to be considered to be surprising that, from the large number of known safeners or antidotes capable of antagonizing the damaging effect of a herbicide on the crop plants, it is in particular the compounds of component c′ listed above which are suitable to compensate the damaging effect of substituted cyclic ketoenols on the crop plants almost completely, without negatively affecting the herbicidal activity against the weeds to any considerable extent.

Emphasis may be given here to the particularly advantageous effect of the particularly preferred and most preferred combination partners from component c′, in particular with respect to sparing cereal plants, such as, for example, wheat, barley and rye, but also maize and rice, as crop plants.

Using, for example according to process (A) ethyl N-[(2-ethyl-4,6-dimethyl)phenylacetyl]prolinate as starting material, the course of the process according to the invention can be represented by the reaction scheme below:

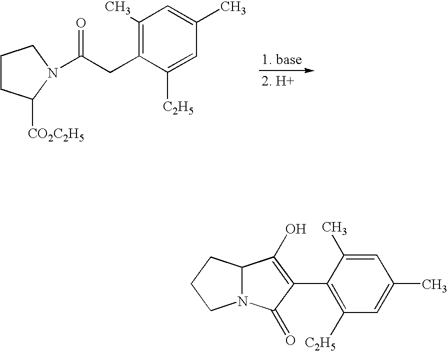

Using, for example, according to process (Bα) 3-[(2-ethyl-4,6-dimethyl)phenyl]-5-ethyl-5-methylpyrrolidine-2,4-dione and pivaloyl chloride as starting materials, the course of the process according to the invention can be represented by the reaction scheme below:

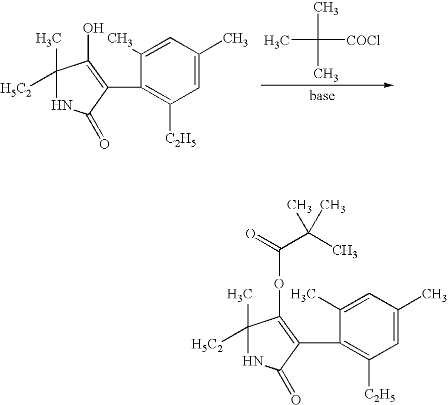

Using, for example, according to process (B) (variant β) 3-[(2-ethyl-4,6-dimethyl)phenyl]-5-propyl-5-methylpyrrolidine-2,4-dione and acetic anhydride as starting materials, the course of the process according to the invention can be represented by the reaction scheme below:

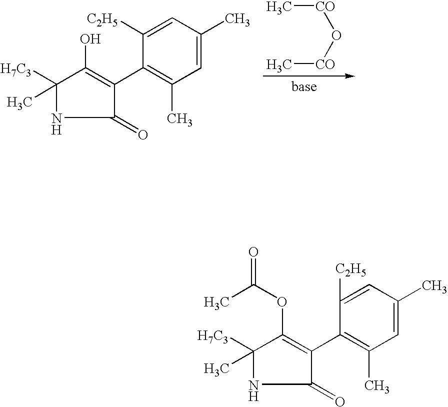

Using, for example, according to process (C) 3-[(2-ethyl-4,6-dimethyl)phenyl]-1,5-tetramethylenepyrrolidone-2,4-dione and ethyl chloroformate as starting materials, the course of the process according to the invention can be represented by the reaction scheme below:

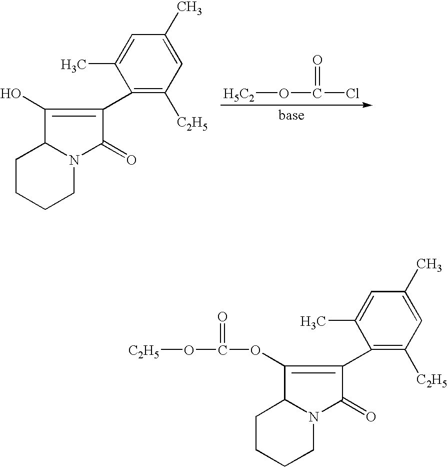

Using, for example, according to process (D), variant α, 3-[(2-ethyl-4,6-dimethyl)phenyl]-5-ethyl-5-methylpyrrolidone-2,4-dione and methyl chloromonothioformate as starting materials, the course of the reaction can be represented as follows:

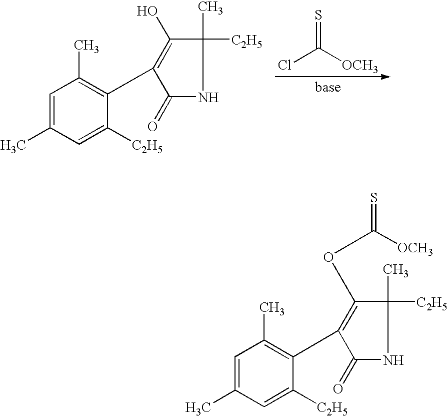

Using, for example, according to process (D), variant β, 3-[(2-ethyl-4,6-dimethyl)phenyl]-5-isopropyl-5-methylpyrrolidine-2,4-dione, carbon disulfide and methyl iodide as starting materials, the course of the reaction can be represented as below:

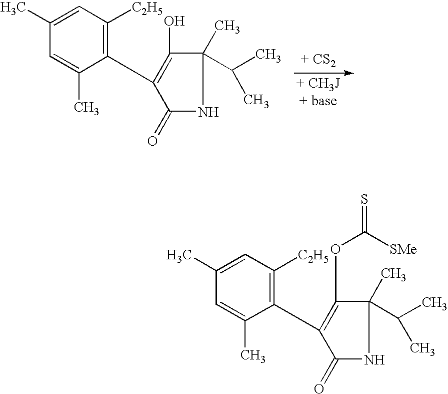

Using, for example, according to process (E) 3-[(2-ethyl-4,6-dimethyl)phenyl]-1,5-trimethylenepyrrolidine-2,4-dione and methanesulfonyl chloride as starting materials, the course of the reaction can be represented by the reaction scheme below:

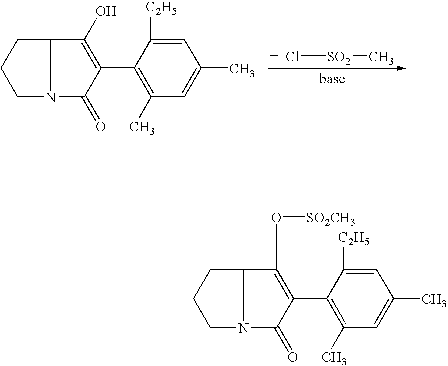

Using, for example, according to process (F) 3-[(2-ethyl-4,6-dimethyl)phenyl]-5-isopropyl-5-methylpyrrolidine-2,4-dione and 2,2,2-trifluoroethyl methanethiophosphonyl chloride as starting materials, the course of the reaction can be represented by the reaction scheme below:

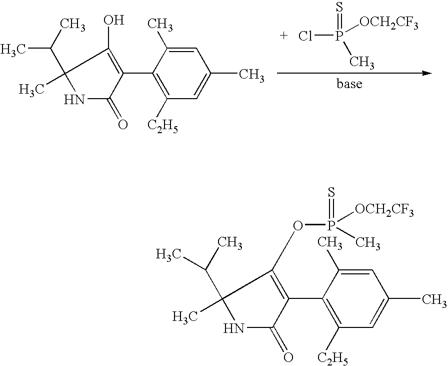

Using, for example, according to process (G) 3-[(2-ethyl-4,6-dimethyl)phenyl]-5-cyclopropyl-5-methylpyrrolidine-2,4-dione and NaOH as components, the course of the process according to the invention can be represented by the reaction scheme below:

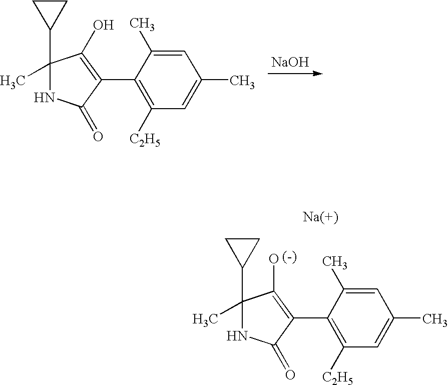

Using, for example, according to process (H), variant α, 3-[(2-ethyl-4,6-dimethyl)phenyl]-5-propyl-5-methylpyrrolidone-2,4-dione and ethyl isocyanate as starting materials, the course of the reaction can be represented by the reaction below:

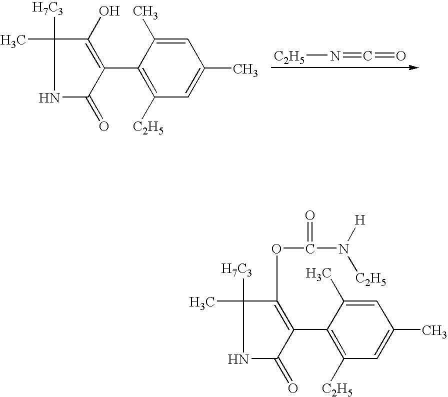

Using, for example, according to process (H), variant β, 3-[(2-ethyl-4,6-dimethyl)phenyl]-5-cyclopropyl-5-methylpyrrolidine-2,4-dione and dimethylcarbamoyl chloride as staring materials, the course of the reaction can be represented by the scheme below:

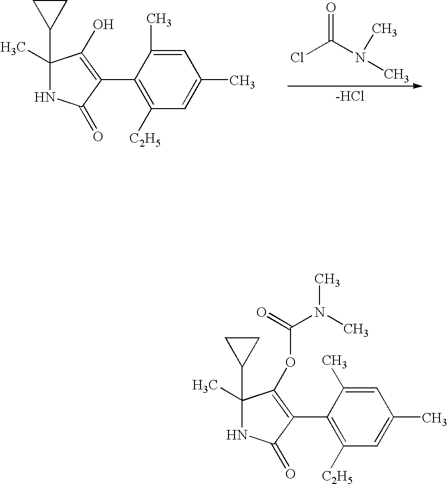

The compounds, required as starting materials in the process (A) according to the invention, of the formula (II), 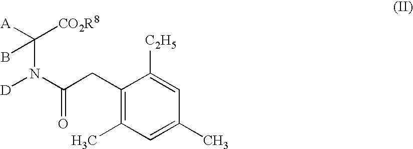
in which
D, A, B and R⁸ are as defined above,
   where D≠H, are novel.

Some compounds where D=H are known from WO 01/74770, or they can be prepared by the processes described therein.

The acylamino acid esters of the formula (II) are obtained, for example, when amino acid derivatives of the formula (XIV), 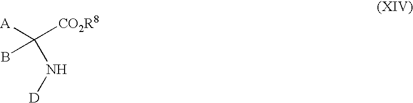
in which
D, A, B and R⁸ are as defined above and D≠H
   are acylated with 2-ethyl-4,6-dimethylphenylacetic acid derivatives of the formula (XV), 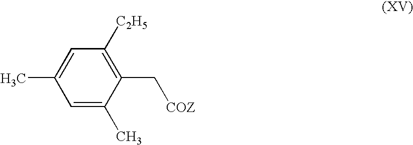
   in which
Z is a leaving group introduced by reagents which activate carboxylic acids, such as carbonyldiimidazole, carbonyldiimides (such as, for example, dicyclohexylcarbodiimide), phosphorylating agents (such as, for example, POCl₃, BOP—Cl), halogenating agents, such as, for example, thionyl chloride, oxalyl chloride, phosgene or chloroformic esters,
   (Chem. Reviews 52, 237-416 (1953); Bhattacharya, Indian J. Chem. 6, 341-5, 1968, patent literature cited at the outset, for example WO 01/74770)
   or when acylamino acids of the formula (XVI), 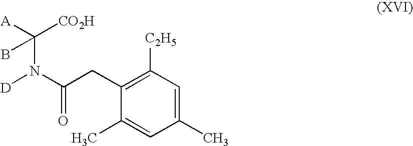
   in which
D, A and B are as defined above, and D≠H
   are esterified (Chem. Ind. (London) 1568 (1968)).

The compounds of the formula (XVI), 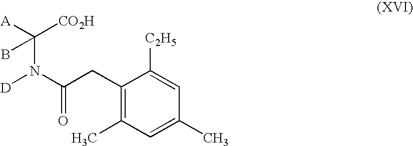
in which
D, A and B are as defined above, and D≠H
   are likewise novel.

The compounds of the formula (XVI) are obtained when amino acids of the formula (XVII), 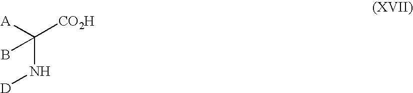
in which
D, A and B are as defined above, and D≠H,
   are acylated according to Schotten-Baumann with 2-ethyl-4,6-dimethylphenylacetoc acid derivatives of the formula (XV), 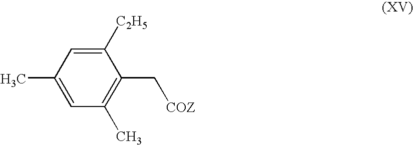
   in which
Z is as defined above,
   (Organikum, VEB Deutscher Verlag der Wissenschaften, Berlin 1977, p. 505).

The compounds of the formula (XV) are obtained, for example, by reacting substituted 2-ethyl-4,6-dimethylphenylacetic acids of the formula (XVIII) 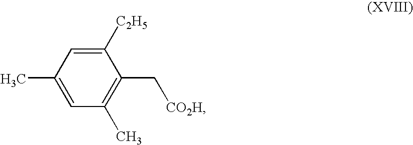
known from WO 01/74770,
with halogenating agents (for example thionyl chloride, thionyl bromide, oxalyl chloride, phosgene, phosphorus trichloride, phosphorus tribromide or phosphorus pentachloride), phosphonylating agents (such as, for example, POCl₃, BOP—Cl), carbonyldiimidazole, carbonyldiimides (for example dicyclohexylcarbonyldiimide), if appropriate in the presence of a diluent (for example optionally chlorinated aliphatic or aromatic hydrocarbons, such as toluene or methylene chloride, or ethers, for example tetrahydrofuran, dioxane, methyl tert-butyl ether), at temperature of from −20° C. to 150° C., preferably from −10° C. to 100° C.

The compound of the formula (XVIII) can furthermore, in principle, be prepared analogously to G. C. Lloyd-Jones, Angew. Chem. Int. Ed. 2002, 41, 953-956; S. L. Buchwald, W. A. Moradi, JACS 2001, 123, 7996-8002 and S. Lee, N. A. Beare, J. F. Hartwig, JACS 2001, 123, 8410-8411:

The process is characterized in that 2-ethyl-4,6-dimethylbromobenzene is reacted with tert-butyl acetate, if appropriate in the presence of a diluent, if appropriate in the presence of a base, if appropriate in the presence of a phosphine ligand and if appropriate in the presence of palladium(0) compounds, and then reacted with an acid.

Suitable diluents for the process according to the invention are all solvents which are inert towards the reagents. Preference is given to using hydrocarbons, such as benzine, benzene, toluene, xylene and tetraline, furthermore ethers, such as diethyl ether, dimethoxyethane, tetrahydrofuran and dioxane.

Suitable bases for the reaction according to the process according to the invention are lithium amide bases, preferably lithium hexyldisilazide, lithium diisopropylamide, lithium dicyclohexylamide.

Suitable phosphine ligands are, for example, tri-tert-butylphosphine and 2-dicyclohexyl-phosphino(2′-N,N-dimethylamino)biphenyl, in particular.

A palladium(0) compound which may be mentioned is, for example, bis(dibenzylideneacetone)palladium.

Acids which may be mentioned are organic acids, for example formic acid, or inorganic acids, for example hydrochloric acid and sulphuric acid.

The reaction temperature in the process according to the invention can be varied within a relatively wide range. In general, the process is carried out at temperatures between −80° C. and +150° C., preferably between 0° C. and 100° C.

When carrying out the process according to the invention, the starting materials are generally each employed in approximately equivalent amounts.

Some of the compounds of the formulae (XIV) and (XVII) are commercially available, some are known, and/or they can be prepared by known processes (see, for example, Compagnon, Miocque Ann. Chim. (Paris) [14] 5, pp. 11-22, 23-27 (1970)).

Some of the compounds, also required for processes (B) to (H), of the formula (I-a′) in which A and B are as defined above are known from WO 01/74770, or they can be prepared by the processes described therein.

The acid halides of the formula (III), carboxylic anhydrides of the formula (IV), chloroformic esters or chloroformic thioesters of the formula (V), chloromonothioformic esters or chlorodithioformic esters of the formula (VI), alkyl halides of the formula (VII), sulphonyl chlorides of the formula (VIII), phosphorus compounds of the formula (IX) and metal hydroxides, metal alkoxides or amines of the formulae (X) and (XI), respectively, and isocyanates of the formula (XII) and carbamoyl chlorides of the formula (XIII) furthermore required as starting materials for carrying out the processes (B), (C), (D), (E), (F), (G) and (H) according to the invention are generally known compounds of organic or inorganic chemistry.

In addition, the compounds of the formulae (XIV) and (XVII) are known from the patent applications cited at the outset and/or can be prepared by the methods given therein.

The process (A) is characterized in that compounds of the formula (II) in which A, B and R⁸ are as defined above and D≠H are subjected to an intramolecular condensation in the presence of a diluent and in the presence of a base.

Suitable diluents for the process (A) according to the invention are all organic solvents which are inert towards the reactants. Preference is given to using hydrocarbons, such as toluene and xylene, furthermore ethers, such as dibutyl ether, tetrahydrofuran, dioxane, glycol dimethyl ether and diglycol dimethyl ether, moreover polar solvents, such as dimethyl sulphoxide, sulpholane, dimethyl-formamide and N-methylpyrrolidone, and also alcohols, such as methanol, ethanol, propanol, isopropanol, butanol, isobutanol and tert-butanol.

Suitable bases (deprotonating agents) for carrying out the process (A) according to the invention are all customary proton acceptors. Preference is given to using alkali metal and alkaline earth metal oxides, hydroxides and carbonates, such as sodium hydroxide, potassium hydroxide, magnesium oxide, calcium oxide, sodium carbonate, potassium carbonate and calcium carbonate, which can also be used in the presence of phase-transfer catalysts, such as, for example, triethylbenzylammonium chloride, tetrabutylammonium bromide, Adogen 464 (=methyltrialkyl(C₈-C₁₀)ammonium chloride) or TDA 1 (=tris(methoxyethoxyethyl)amine). It is furthermore possible to use alkali metals, such as sodium or potassium. Also suitable are alkali metal and alkaline earth metal amides and hydrides, such as sodium amide, sodium hydride and calcium hydride, and additionally also alkali metal alkoxides, such as sodium methoxide, sodium ethoxide and potassium tert-butoxide.

When carrying out the process (A) according to the invention, the reaction temperature can be varied within a relatively wide range. In general, the process is carried out at temperatures between 0° C. and 250° C., preferably between 50° C. and 150° C.

The process (A) according to the invention is generally carried out under atmospheric pressure.

When carrying out the process (A) according to the invention, the reaction component of the formula (II) and the deprotonating base are generally employed in equimolar to about doubly equimolar amounts. However, it is also possible to use a relatively large excess (up to 3 mol) of one component or the other.

The process (Bα) is characterized in that compounds of the formula (I-a) or (I-a′) are in each case reacted with carbonyl halides of the formula (III), if appropriate in the presence of a diluent and if appropriate in the presence of an acid binder.

Suitable diluents for the process (Bα) according to the invention are all solvents which are inert towards the acid halides. Preference is given to using hydrocarbons, such as benzine, benzene, toluene, xylene and tetralin, furthermore halogenated hydrocarbons, such as methylene chloride, chloroform, carbon tetrachloride, chlorobenzene and o-dichlorobenzene, moreover ketones, such as acetone and methyl isopropyl ketone, furthermore ethers, such as diethyl ether, tetrahydrofuran and dioxane, additionally carboxylic esters, such as ethyl acetate, and also strongly polar solvents, such as dimethyl sulphoxide and sulpholane. The hydrolytic stability of the acid halide permitting, the reaction can also be carried out in the presence of water.

Suitable acid binders for the reaction according to process (Bα) according to the invention are all customary acid acceptors. Preference is given to using tertiary amines, such as triethylamine, pyridine, diazabicyclooctane (DABCO), diazabicycloundecene (DBU), diazabicyclononene (DBN), Hünig base and N,N-dimethylaniline, furthermore alkaline earth metal oxides, such as magnesium oxide and calcium oxide, moreover alkali metal and alkaline earth metal carbonates, such as sodium carbonate, potassium carbonate and calcium carbonate, and also alkali metal hydroxides, such as sodium hydroxide and potassium hydroxide.

In the process (Bα) according to the invention, the reaction temperature can be varied within a relatively wide range. In general, the process is carried out at temperatures between −20° C. and +150° C., preferably between 0° C. and 100° C.

When carrying out the process (Bα) according to the invention, the starting materials of the formula (I-a) or (I-a′) and the carbonyl halide of the formula (III) are generally each employed in approximately equivalent amounts. However, it is also possible to use a relatively large excess (up to 5 mol) of the carbonyl halide. Work-up is carried out by customary methods.

The process (Bβ) is characterized in that compounds of the formula (I-a) or (I-a′) are in each case reacted with carboxylic anhydrides of the formula (IV), if appropriate in the presence of a diluent and if appropriate in the presence of an acid binder.

Suitable diluents for the process (Bβ) according to the invention are preferably those diluents which are also preferred when using acid halides. Furthermore, excess carboxylic anhydride may simultaneously act as diluent.

The acid binders which are added, if appropriate, in the process (Bβ) are preferably those acid binders which are also preferred when using acid halides.

The reaction temperature in the process (Bβ) according to the invention may be varied within a relatively wide range. In general, the process is carried out at temperatures between −20° C. and +150° C., preferably between 0° C. and 100° C.

When carrying out the process (Bβ) according to the invention, the starting materials of the formula (I-a) or (I-a′) and the carboxylic anhydride of the formula (IV) are generally each employed in approximately equivalent amounts. However, it is also possible to use a relatively large excess (up to 5 mol) of the carboxylic anhydride. Work-up is carried out by customary methods.

In general, diluent and excess carboxylic anhydride and the carboxylic acid formed are removed by distillation or by washing with an organic solvent or with water.

The process (C) is characterized in that compounds of the formula (I-a) or (I-a′) are in each case reacted with chloroformic esters or chloroformic thioesters of the formula (V) if appropriate in the presence of a diluent and if appropriate in the presence of an acid binder.

Suitable acid binders for the process (C) according to the invention are all customary acid acceptors. Preference is given to using tertiary amines, such as triethylamine, pyridine, DABCO, DBU, DBA, Hünig base and N,N-dimethylaniline, furthermore alkaline earth metal oxides, such as magnesium oxide and calcium oxide, moreover alkali metal and alkaline earth metal carbonates, such as sodium carbonate, potassium carbonate and calcium carbonate, and also alkali metal hydroxides, such as sodium hydroxide and potassium hydroxide.

Suitable diluents for the process (C) according to the invention are all solvents which are inert towards the chloroformic esters or chloroformic thioesters. Preference is given to using hydrocarbons, such as benzine, benzene, toluene, xylene and tetralin, furthermore halogenated hydrocarbons, such as methylene chloride, chloroform, carbon tetrachloride, chlorobenzene and o-dichlorobenzene, moreover ketones, such as acetone and methyl isopropyl ketone, furthermore ethers, such as diethyl ether, tetrahydrofuran and dioxane, additionally carboxylic esters, such as ethyl acetate, and also strongly polar solvents, such as dimethyl sulphoxide and sulpholane.

When carrying out the process (C) according to the invention, the reaction temperature can be varied within a relatively wide range. In general, the reaction temperature is between −20° C. and +100° C., preferably between 0° C. and 50° C.

The process (C) according to the invention is generally carried out under atmospheric pressure.

When carrying out the process (C) according to the invention, the starting materials of the formula (I-a) or (I-a′) and the appropriate chloroformic ester or chloroformic thioester of the formula (V) are generally each employed in approximately equivalent amounts. However, it is also possible to use a relatively large excess (up to 2 mol) of one component or the other. Work-up is carried out by customary methods. In general, precipitated salts are removed and the reaction mixture which remains is concentrated by removing the diluent under reduced pressure.

The process (D) according to the invention is characterized in that compounds of the formula (I-a) or (I-a′) are in each case reacted with (Dα) compounds of the formula (VI) in the presence of a diluent and, if appropriate, in the presence of an acid binder or (Dβ) carbon disulphide and then with alkyl halides of the formula (VII), if appropriate in the presence of a diluent and if appropriate in the presence of a base.

In preparation process (Dα), about 1 mol of chloromonothioformic ester or chlorodithioformic ester of the formula (VI) is reacted per mole of starting material of the formula (I-a) or (I-a′), at from 0 to 120° C., preferably from 20 to 60° C.

Suitable diluents, which are added, if appropriate, are all inert polar organic solvents, such as ethers, esters, amides, sulphones, sulphoxides, but also halogenated alkanes.

Preference is given to using dimethyl sulphoxide, ethyl acetate, tetaahydrofuran, dimethylformamide or methylene chloride.

If, in a preferred embodiment, the enolate salt of the compounds (I-a) or (I-a′) is prepared by adding strong deprotonating agents, such as, for example, sodium hydride or potassium tert-butoxide, the further addition of acid binders may be dispensed with.

If acid binders are used, these are customary inorganic or organic bases, for example sodium hydroxide, sodium carbonate, potassium carbonate, pyridine and triethylamine.

The reaction can be carried out at atmospheric pressure or under elevated pressure and is preferably carried out at atmospheric pressure. Work-up is carried out by customary methods.

In preparation process (Dβ), in each case the equimolar amount or an excess of carbon disulphide is added per mole of starting material of the formula (I-a) or (I-a′). The process is preferably carried out at temperatures of from 0 to 50° C. and in particular at from 20 to 30° C.

Frequently, it is expedient to prepare initially the corresponding salt from the compounds of the formula (I-a) by adding a base (such as, for example, potassium tert-butoxide or sodium hydride). In each case, the compound (I-a) is reacted with carbon disulphide until the formation of the intermediate has ended, for example after several hours of stirring at room temperature.

Suitable bases for the process (Dβ) are all customary proton acceptors. Preference is given to using alkali metal hydrides, alkali metal alkoxides, alkali metal and alkaline earth metal carbonates or bicarbonates or nitrogen bases. Examples which may be mentioned are sodium hydride, sodium methoxide, sodium hydroxide, calcium hydroxide, potassium carbonate, sodium bicarbonate, triethylamine, dibenzylamine, diisopropylethylamine, pyridine, quinoline, diazabicyclooctane (DABCO), diazabicyclononene (DBN) and diazabicycloundecene (DBU).

Suitable diluents are all solvents which are customary for this process.

Preference is given to using aromatic hydrocarbons, such as benzene or toluene, alcohols, such as methanol, ethanol, isopropanol or ethylene glycol, nitrites, such as acetonitrile, ethers, such as tetrahydrofuran or dioxane, amides, such as dimethylformamide, or other polar solvents, such as dimethyl sulphoxide or sulpholane.

The further reaction with the alkyl halide of the formula (VII) is preferably carried out at from 0 to 70° C. and in particular at from 20 to 50° C. Here, at least an equimolar amount of alkyl halide is used.

The process is carried out at atmospheric pressure or under elevated pressure, preferably at atmospheric pressure.

Work-up is again carried out by customary methods.

The process (E) according to the invention is characterized in that compounds of the formula (I-a) or (I-a′) are in each case reacted with sulphonyl chlorides of the formula (VIII), if appropriate in the presence of a diluent and if appropriate in the presence of an acid binder.

In the preparation process (E), about 1 mol of sulphonyl chloride of the formula (VIII) is reacted per mole of starting material of the formula (I-a) or (I-a′), at from −20 to 150° C., preferably from 20 to 70° C.

The process (E) is preferably carried out in the presence of a diluent.

Suitable diluents are all inert polar organic solvents, such as ethers, esters, amides, nitrites, sulphones, sulphoxides or halogenated hydrocarbons, such as methylene chloride.

Preference is given to using dimethyl sulphoxide, tetrahydrofuran, ethyl acetate, dimethylformamide, methylene chloride.

If, in a preferred embodiment, the enolate salt of the compounds (I-a) or (I-a′) is prepared by adding strong deprotonating agents (such as, for example, sodium hydride or potassium tert-butoxide), the further addition of acid binders may be dispensed with.

If acid binders are used, these are customary inorganic or organic bases, for example sodium hydroxide, sodium carbonate, potassium carbonate, pyridine and triethylamine.

The reaction can be carried out at atmospheric pressure or under elevated pressure and is preferably carried out at atmospheric pressure. Work-up is carried out by customary methods.

The process (F) according to the invention is characterized in that compounds of the formula (I-a) or (I-a′) are in each case reacted with phosphorus compounds of the formula (IX), if appropriate in the presence of a diluent and if appropriate in the presence of an acid binder.

In preparation process (F), to obtain compounds of the formula (I-e), 1 to 2, preferably 1 to 1.3, mol of the phosphorus compound of the formula (IX) are reacted per mole of the compounds (I-a) or (I-a′), at temperatures between −40° C. and 150° C., preferably between −10 and 110° C.

The process (F) is preferably carried out in the presence of a diluent.

Suitable diluents are all inert polar organic solvents, such as ethers, esters, amides, nitriles, sulphides, sulphones, sulphoxides, etc.

Preference is given to using acetonitrile, ethyl acetate, dimethyl sulphoxide, tetrahydrofuran, dimethylformamide, methylene chloride.

Suitable acid binders, which are added if appropriate, are customary inorganic or organic bases, such as hydroxides, carbonates or amines. Examples which may be mentioned are sodium hydroxide, sodium carbonate, potassium carbonate, pyridine and triethylamine.

The reaction can be carried out at atmospheric pressure or under elevated pressure and is preferably carried out at atmospheric pressure. Work-up is carried out by customary methods of organic chemistry. The end products are preferably purified by crystallization, chromatographic purification or by “incipient distillation”, i.e. removal of the volatile components under reduced pressure.

The process (G) is characterized in that compounds of the formula (I-a) or (I-a′) are in each case reacted with metal hydroxides or metal alkoxides of the formula (X) or amines of the formula (XI), if appropriate in the presence of a diluent.

Suitable diluents for the process (G) according to the invention are preferably ethers, such as tetrahydrofuran, dioxane, diethyl ether or else alcohols, such as methanol, ethanol, isopropanol, but also water. The process (G) according to the invention is generally carried out under atmospheric pressure. The reaction temperature is generally between −20° C. and 100° C., preferably between 0° C. and 50° C.

The process (H) according to the invention is characterized in that compounds of the formula (I-a) or (I-a′) are in each case reacted with (Hα) compounds of the formula (XII), if appropriate in the presence of a diluent and if appropriate in the presence of a catalyst, or (Hβ) with compounds of the formula (XIII), if appropriate in the presence of a diluent and if appropriate in the presence of an acid binder.

In preparation process (Hα), about 1 mol of isocyanate of the formula (XII) is reacted per mole of starting material of the formula (I-a) or (I-a′), at from 0 to 100° C., preferably at from 20 to 50° C.

The process (Hα) is preferably carried out in the presence of a diluent.

Suitable diluents are all inert organic solvents, such as ethers, esters, amides, nitriles, sulphones or sulphoxides.

If appropriate, catalysts may be added to accelerate the reaction. Suitable for use as catalysts are, very advantageously, organotin compounds, such as, dibutyltin dilaurate.

The process is preferably carried out at atmospheric pressure.

In the preparation process (Hβ), about 1 mol of carbamoyl chloride of the formula (XIII) is reacted per mole of starting material of the formula (I-a) or (I-a′), at from 0 to 150° C., preferably at from 20 to 70° C.

Suitable diluents which are added, if appropriate, are all inert polar organic solvents, such as ethers, esters, amides, sulphones, sulphoxides or halogenated hydrocarbons.

Preference is given to using dimethyl sulphoxide, ethyl acetate, tetrahydrofuran, dimethylformamide or methylene chloride.

If, in a preferred embodiment, the enolate salt of the compounds (I-a) or (I-a′) is prepared by adding strong deprotonating agents (such as, for example, sodium hydride or potassium tert-butoxide), the further addition of acid binders may be dispensed with.

If acid binders are used, these are customary inorganic or organic bases, for example sodium hydroxide, sodium carbonate, potassium carbonate, triethylamine or pyridine.

The reaction can be carried out at atmospheric pressure or under elevated pressure and is preferably carried out at atmospheric pressure. Work-up is carried out by customary methods.

The active compounds are well tolerated by plants and have advantageous toxicity to warm-blooded species; they can be employed for controlling animal pests, in particular insects, arachnids and nematodes encountered in agriculture, forests, in the protection of stored products and materials and in the hygiene sector. They are preferably used as crop protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae and Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp., Damalinia spp.

From the order of the Thysanoptera, for example, Hercinothrips femoralis, Thrips tabaci, Thrips palmi, Frankliniella occidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniaius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp. and Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilacina varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., *Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorphoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

If appropriate, the compounds or active compound combinations according to the invention may also be used in certain concentrations or application rates to act as herbicides. If appropriate, they can also be employed as intermediates or precursors for the synthesis of further active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits and seeds and also roots, tubers and rhizomes. The plant parts also include harvested material, and vegetative and generative propagation material, for example cuttings, tubers, rhizomes, offsets and seeds.

The treatment according to the invention of the plants and plant parts with the active compounds or active compound combinations is carried out directly or by allowing the compounds to act on their surroundings, habitat or storage space by the customary treatment methods, for example by immersion, spraying, atomizing, evaporation, fogging, scattering or painting on and, in the case of propagation material, in particular in the case of seeds, also by applying one or more coats.

The active compounds or active compound combinations can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

Suitable solid carriers are:
for example ammonium salts and ground natural minerals such as kaolins, clays, talc, chalk, quartz, attapulgite, montmorillonite or diatomaceous earth, and ground synthetic materials such as highly-disperse silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates; suitable dispersants are: for example lignin-sulphite waste liquors and methylcellulose.

Tackifiers such as carboxymethylcellulose and natural and synthetic polymers in the form of powders, granules or lattices, such as gum arabic, polyvinyl alcohol and polyvinyl acetate, or else natural phospholipids such as cephalins and lecithins and synthetic phospholipids can be used in the formulations. Other additives can be mineral and vegetable oils.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention, as such or in their formulations, can also be used as a mixture with known fungicides, bactericides, acaricides, nematicides or insecticides, for example in order to widen the spectrum of action or to prevent the development of resistances in this way. In many cases, synergistic effects result, i.e. the activity of the mixture exceeds the activity of the individual components.

Compounds which are suitable as components in the mixtures are, for example, the following:

Fungicides:

aldimorph, ampropylfos, ampropylfos-potassium, andoprim, anilazine, azaconazole, azoxystrobin,
benalaxyl, benodanil, benomyl, benzamacril, benzamacril-isobutyl, bialaphos, binapacryl, biphenyl, bitertanol, blasticidin-S, bromuconazole, bupirimate, buthiobate,
calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,
debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,
edifenphos, epoxiconazole, etaconazole, ethirimol, etridiazole,
famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox,
guazatine,
hexachlorobenzene, hexaconazole, hymexazole,
imazalil, imibenconazole, iminoctadine, iminoctadine albesilate, iminoctadine triacetate, iodocarb, ipconazole, iprobenfos (IBP), iprodione, irumamycin, isoprothiolane, isovaledione,
kasugamycin, kresoxim-methyl, copper preparations, such as: copper hydroxide, copper naphthenate, copper oxychloride, copper sulphate, copper oxide, oxine-copper and Bordeaux mixture,
mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,
nickel dimethyldithiocarbamate, nitrothal-isopropyl, nuarimol,
ofurace, oxadixyl, oxamocarb, oxolinic acid, oxycarboxim, oxyfenthiin,
paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur,
quinconazole, quintozene (PCNB),
sulphur and sulphur preparations,
tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole,
uniconazole,
validamycin A, vinclozolin, viniconazole,
zarilamide, zineb, ziram and also
Dagger G,
OK-8705,
OK-8801,
α-(1,1-dimethylethyl)-β-(2-phenoxyethyl)-1H-1,2,4-triazole-1-ethanol,
α-(2,4-dichlorophenyl)-β-fluoro-β-propyl-1H-1,2,4-triazole-1-ethanol,
α-(2,4-dichlorophenyl)-β-methoxy-α-methyl-1H-1,2,4-triazole-1-ethanol,
α-(5-methyl-1,3-dioxan-5-yl)-β-[[4-(trifluoromethyl)phenyl]methylene]-1H-1,2,4-triazole-1-ethanol,
(5RS,6RS)-6-hydroxy-2,2,7,7-tetramethyl-5-(1H-1,2,4-triazol-1-yl)-3-octanone,
(E)-α-(methoxyimino)-N-methyl-2-phenoxyphenylacetamide,
1-isopropyl{2-methyl-1-[[[1-4-methylphenyl)ethyl]amino]carbonyl]propyl}carbamate,
1-(2,4-dichlorophenyl)-2-(1H-1,2,4-triazol-1-yl)ethanone O-(phenylmethyl)oxime,
1-(2-methyl-1-naphthalenyl)-1H-pyrrole-2,5-dione,
1-(3,5-dichlorophenyl)-3-(2-propenyl)-2,5-pyrrolidinedione,
1-[(diiodomethyl)sulphonyl]4-methylbenzene,
1-[[2-(2,4-dichlorophenyl)-1,3-dioxolan-2-yl]methyl]-1H-imidazole,
1-[[2-(4-chlorophenyl)-3-phenyloxiranyl]methyl]-1H-1,2,4-triazole,
1-[1-[2-[(2,4-dichlorophenyl)methoxy]phenyl]ethenyl]-1H-imidazole,
1-methyl-5-nonyl-2-(phenylmethyl)-3-pyrrolidinol,
2′,6′-dibromo-2-methyl-4′-trifluoromethoxy-4′-trifluoromethyl-1,3-thiazole-5-carboxanilide,
2,2-dichloro-N-[1-(4-chlorophenyl)ethyl]-1-ethyl-3-methylcyclopropanecarboxamide,
2,6-dichloro-5-(methylthio)-4-pyrimidinyl thiocyanate,
2,6-dichloro-N-(4-trifluoromethylbenzyl)benzamide,
2,6-dichloro-N-[[4-(trifluoromethyl)phenyl]methyl]benzamide;
2-(2,3,3-triiodo-2-propenyl)-2H-tetrazole,
2-[(1-methylethyl)sulphonyl]-5-(trichloromethyl)-1,3,4-thiadiazole,
2-[[6-deoxy-4-O-(4-O-methyl-β-D-glycopyranosyl)-α-D-glucopyranosyl]amino]-4-methoxy-1H-pyrrolo[2,3-d]pyrimidine-5-carbonitrile,
2-aminobutane,
2-bromo-2-(bromomethyl)pentanedinitrile,
2-chloro-N-(2,3-dihydro-1,1,3-trimethyl-1H-inden-4-yl)-3-pyridinecarboxamide,
2-chloro-N-(2,6-dimethylphenyl)-N-(isothiocyanatomethyl)acetamide,
2-phenylphenol (OPP),
3,4-dichloro-1-[4-(difluoromethoxy)phenyl]-1H-pyrrole-2,5-dione,
3,5-dichloro-N-[cyano-[(1-methyl-2-propynyl)oxy]methyl]benzamide,
3-(1,1-dimethylpropyl)-1-oxo-1H-indene-2-carbonitrile,
3-[2-(4-chlorophenyl)-5-ethoxy-3-isoxazolidinyl]pyridine,
4-chloro-2-cyano-N,N-dimethyl-5-(4-methylphenyl)-1H-imidazole-1-sulphonamide,
4-methyltetrazolo[1,5-a]quinazolin-5(4H)-one,
8-(1,1-dimethylethyl)-N-ethyl-N-propyl-1,4-dioxaspiro[4.5]decane-2-methanamine,
8-hydroxyquinoline sulphate,
9H-xanthene-2-[(phenylamino)carbonyl]-9-carboxylic hydrazide,
bis-(1-methylethyl)-3-methyl-4-[(3-methylbenzoyl)oxy]-2,5-thiophene dicarboxylate,
cis-1-(4-chlorophenyl)-2-(1H-1,2,4-triazol-1-yl)cycloheptanol,
cis-4-[3-[4-(1,1-dimethylpropyl)phenyl-2-methylpropyl]-2,6-dimethylmorpholine hydrochloride,
ethyl [(4-chlorophenyl)azo]cyanoacetate,
potassium bicarbonate,
methanetetrathiol sodium salt,
methyl 1-(2,3-dihydro-2,2-dimethyl-1H-inden-1-yl)-1H-imidazole-5-carboxylate,
methyl N-(2,6-dimethylphenyl)-N-(5-isoxazolylcarbonyl)-DL-alaninate,
methyl N-(chloroacetyl)-N-(2,6-dimethylphenyl)-DL-alaninate,
N-(2,3-dichloro-4-hydroxyphenyl)-1-methylcyclohexanecarboxamide,
N-(2,6-dimethylphenyl)-2-methoxy-N-(tetrahydro-2-oxo-3-furanyl)acetamide,
N-(2,6-dimethylphenyl)-2-methoxy-N-(tetrahydro-2-oxo-3-thienyl)acetamide,
N-(2-chloro-4-nitrophenyl)-4-methyl-3-nitrobenzenesulphonamide,
N-(4-cyclohexylphenyl)-1,4,5,6-tetrahydro-2-pyrimidinamine,
N-(4-hexylphenyl)-1,4,5,6-tetrahydro-2-pyrimidinamine,
N-(5-chloro-2-methylphenyl)-2-methoxy-N-(2-oxo-3-oxazolidinyl)acetamide,
N-(6-methoxy)-3-pyridinylcyclopropanecarboxamide,
N-[2,2,2-trichloro-1-[(chloroacetyl)amino]ethyl]benzamide,
N-[3-chloro-4,5-bis(2-propinyloxy)phenyl]-N′-methoxymethanimidamide,
N-formyl-N-hydroxy-DL-alanine sodium salt,
O,O-diethyl [2-(dipropylamino)-2-oxoethyl]ethylphosphoramidothioate,
O-methyl S-phenyl phenylpropylphosphoramidothioate,
S-methyl 1,2,3-benzothiadiazole-7-carbothioate,
spiro[2H]-1-benzopyrane-2,1′(3′H)-isobenzofuran-3′-one,
4-[(3,4-dimethoxyphenyl)-3-(4-fluorophenyl)acryloyl]morpholine.
   Bactericides:
bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other.copper preparations.
   Insecticides/acaricides/nematicides:
abamectin, acephate, acetamiprid, acrinathrin, alanycarb, aldicarb, aldoxycarb, alphacypermethrin, alphamethrin, amitraz, avermectin, AZ 60541, azadirachtin, azamethiphos, azinphos A, azinphos M, azocyclotin,
Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben,
cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine,
deltamethrin, demeton M, demeton S, demeton-S-mrethyl, diafenthiuron, diazinon, dichlorvos, dicofol, diflubenzuron, dimethoate, dimethylvinphos, diofenolan, disulfoton, docusat-sodium, dofenapyn,
eflusilanate, emamectin, empenthrin, endosulfan, Entomopfthora spp., esfenvalerate, ethiofencarb, ethion, ethoprophos, etofenprox, etoxazole, etrimfos,
fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluozinam, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb,
granulosis viruses,
halofenozide, HCH, heptenophos, hexaflumuron, hexythiazox, hydroprene,
imidacloprid, indoxacarb, isazofos, isofenphos, isoxathion, ivermectin,
nuclear polyhedrosis viruses,
lambda-cyhalothrin, lufenuron,
malathion, mecarbam, metaldehyde, methamidophos, Metharhizium anisopliae, Metharhizium flavoviride, methidathion, methiocarb, methoprene, methomyl, methoxyfenozide, metolcarb, metoxadiazone, mevinphos, milbemectin, milbemycin, monocrotophos,
naled, nitenpyram, nithiazine, novaluron,
omethoate, oxamyl, oxydemethon M,
Paecilomyces fumosoroseus, parathion A, parathion M, permethrin, phenthoate, phorate, phosalone, phosmet, phosphamidon, phoxim, pirimicarb, pirimiphos A, pirimiphos M, profenofos, promecarb, propargite, propoxur, prothiofos, prothoate, pymetrozine, pyraclofos, pyresmethrin, pyrethrum, pyridaben, pyridathion, pyrimidifen, pyriproxyfen,
quinalphos,
ribavirin,
salithion, sebufos, silafluofen, spinosad, spirodiclofen, sulfotep, sulprofos,
tau-fluvalinate, tebufenozide, tebufenpyrad, tebupirimiphos, teflubenzuron, tefluthrin, temephos, temivinphos, terbufos, tetrachlorvinphos, tetradifon, theta-cypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazurone, trichlophenidine, trichlorfon, triflumuron, trimethacarb,
vamidothion, vaniliprole, Verticillium lecanii,
YI 5302,
zeta-cypermethrin, zolaprofos
(1R-cis)-[5-(phenylmethyl)-3-furanyl]methyl 3-[(dihydro-2-oxo-3 (2H)-furanylidene)methyl]-2,2-dimethylcyclopropanecarboxylate,
(3-phenoxyphenyl)methyl 2,2,3,3-tetramethylcyclopropanecarboxylate,
1-[(2-chloro-5-thiazolyl)methyl]tetrahydro-3,5-dimethyl-N-nitro-1,3,5-triazine-2(1H)-imine,
2-(2-chloro-6-fluorophenyl)-4-[4-(1,1-dimethylethyl)phenyl]4,5-dihydrooxazole,
2-(acetyloxy)-3-dodecyl-1,4-naphthalenedione,
2-chloro-N-[[[4-(1-phenylethoxy)phenyl]amino]carbonyl]benzamide,
2-chloro-N-[[[4-(2,2-dichloro-1,1-difluoroethoxy)phenyl]amino]carbonyl]benzamide,
3-methylphenyl propylcarbamate,
4-[4-(4-ethoxyphenyl)-4-methylpentyl]-1-fluoro-2-phenoxybenzene,
4-chloro-2-(1,1-dimethylethyl)-5-[[2-(2,6-dimethyl-4-phenoxyphenoxy)ethyl]thio]-3(2H)-pyridazinone,
4-chloro-2-(2-chloro-2-methylpropyl)-5-[(6-iodo-3-pyridinyl)methoxy]-3(2H)-pyridazin,
4-chloro-5-[(6-chloro-3-pyridinyl)methoxy]-2-(3,4-dichlorophenyl)-3(2H)-pyridazinone,
Bacillus thuringiensis strain EG-2348,
[2-benzoyl-1-(1,1-dimethylethyl)]hydrazinobenzoic acid,
2,2-dimethyl-3-(2,4-dichlorophenyl)-2-oxo-1-oxaspiro[4.5]dec-3-en-4-yl butanoate,
[3-[(6-chloro-3-pyridinyl)methyl]-2-thiazolidinylidene]cyanamide,
dihydro-2-(nitromethylene)-2H-1,3-thiazine-3(4H)-carboxaldehyde,
ethyl [2-[[1,6-dihydro-6-oxo-1-(phenylmethyl)-4-pyridazinyl]oxy]ethyl]carbamate,
N-(3,4,4-trifluoro-1-oxo-3-butenyl)glycine,
N-(4-chlorophenyl)-3-[4-(difluoromethoxy)phenyl]4,5-dihydro-4-phenyl-1H-pyrazole-1-carboxamide,
N-[(2-chloro-5-thiazolyl)methyl]-N′-methyl-N″-nitroguanidine,
N-methyl-N′-(1-methyl-2-propenyl)-1,2-hydrazinedicarbothioamide,
N-methyl-N′-2-propenyl-1,2-hydrazinedicarbothioamide,
O,O-diethyl [2-(dipropylamino)-2-oxoethyl]ethylphosphoramidothioate,
N-cyanomethyl-4-trifluoromethylnicotinamide,
3,5-dichloro-1-(3,3-dichloro-2-propenyloxy)-4-[3-(5-trifluoromethylpyridin-2-yloxy)propoxy]benzene.

A mixture with other known active compounds, such as herbicides, or with fertilizers and growth regulators is also possible.

When used as insecticides in their commercially available formulations and in the use forms prepared with these formulations, the active compounds according to the invention can furthermore exist in the form of a mixture with synergists. Synergists are compounds by which the activity of the active compounds is increased without it being necessary for the synergist added to be active itself.

The active compound content of the use forms prepared from the commercially available formulations can vary within wide ranges. The active compound concentration of the use forms can be from 0.0000001 up to 95% by weight of active compound, preferably between 0.0001 and 1% by weight.

They are applied in a customary manner adapted to suit the use forms.

When used against hygiene pests and pests of stored products, the active compound or active compound combinations is/are distinguished by excellent residual action on wood and clay as well as good stability to alkali on limed substrates.

As already mentioned above, it is possible to treat all plants or their parts in accordance with the invention. In a preferred embodiment, wild plant species or plant varieties and plant cultivars which have been obtained by traditional biological breeding methods, such as hybridization or protoplast fusion, and the parts of these varieties and cultivars are treated. In a further preferred embodiment, transgenic plants and plant cultivars which have been obtained by recombinant methods, if appropriate in combination with conventional methods (genetically modified organisms), and their parts are treated. The term “parts” or “parts of plants” or “plant parts” has been explained above.

Plants which are treated particularly preferably in accordance with the invention are those of the plant cultivars which are in each case commercially available or in use. Plant cultivars are understood as meaning plants with new traits which have been bred either by conventional breeding, by mutagenesis or by recombinant DNA techniques. They may take the form of cultivars, biotypes and genotypes.

Depending on the plant species or plant cultivars, their location and growth conditions (soils, climate, vegetation period, nutrition), the treatment according to the invention may also result in superadditive (“synergistic”) effects. Thus, for example, reduced application rates and/or a widened activity spectrum and/or an increase in the activity of the substances and compositions which can be used in accordance with the invention, better plant growth, increased tolerance to high or low temperatures, increased tolerance to drought or to salinity in the water or soil, increased flowering performance, facilitated harvesting, accelerated maturation, higher yields, higher quality and/or better nutritional value of the harvested products, better storage characteristics and/or processibility of the harvested products are possible which exceed the effects which were actually to be expected.

The preferred transgenic plants or plant cultivars (those obtained by recombinant methods) to be treated in accordance with the invention include all those plants which, owing to the process of recombinant modification, were given genetic material which confers particular, advantageous, valuable traits to these plants. Examples of such properties are better plant growth, increased tolerance to high or low temperatures, increased tolerance to drought or to salinity in the water or soil, increased flowering performance, facilitated harvesting, accelerated maturation, higher yields, higher quality and/or higher nutritional value of the harvested products, better storage characteristics and/or processibility of the harvested products. Further examples of such traits, examples which must be mentioned especially, are better defence of the plants against animal and microbial pests, such as against insects, mites, phytopathogenic fungi, bacteria and/or viruses and an increased tolerance of the plants to certain herbicidal active compounds. Examples of transgenic plants which may be mentioned are the important crop plants, such as cereals (wheat, rice), maize, soya beans, potato, cotton, oilseed rape, beet, sugar cane and fruit plants (with the fruits apples, pears, citrus fruits and grapes), with particular emphasis on maize, soya beans, potatoes, cotton and oilseed rape. Traits which are especially emphasized are the increased defence of the plants against insects, owing to toxins being formed in the plants, in particular toxins which are generated in the plants by the genetic material of Bacillus thuringiensis (for example by the genes CryIA(a), CryIA(b), CryIA(c), CryIIA, CryIIIA, CryIIIB2, Cry9c Cry2Ab, Cry3Bb and CryIF and their combinations; hereinbelow “Bt plants”). Other traits which are particularly emphasized are the increased defence of plants against fungi, bacteria and viruses by the systemic acquired resistance (SAR), systemin, phytoalexins, elicitors and resistance genes and correspondingly expressed proteins and toxins. Other traits which are especially emphasized are the increased tolerance of the plants to certain herbicidal active compounds, for example imidazolinones, sulphonylureas, glyphosate or phosphinothricin (for example “PAT” gene). The genes which confer the desired traits in each case may also be present in the transgenic plants in combination with one another. Examples of “Bt plants” which may be mentioned are maize cultivars, cotton cultivars, soya bean cultivars and potato cultivars which are commercially available under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize cultivars, cotton cultivars and soya bean cultivars which are commercially available under the trade names Roundup Ready® (tolerance to glyphosate, for example maize, cotton, soya bean), Liberty Link® (tolerance to phosphinothricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include also the varieties commercially available under the name Clearfield® (for example maize). Naturally, these statements also apply to plant cultivars having these genetic traits or genetic traits still to be developed, which plant cultivars will be developed and/or marketed in the future.

The plants listed can be treated particularly advantageously with the compounds according to the invention or the active compound mixtures according to the invention. The preferred ranges stated above for the active compounds and mixtures also apply to the treatment of these plants. Particular emphasis may be given to the treatment of plants with the compounds or mixtures specifically mentioned in the present text.

The active compounds or active compound combinations according to the invention are not only active against plant, hygiene and stored-product pests, but also, in the veterinary medicine sector, against animal parasites (ectoparasites), such as ixodid ticks, argasid ticks, scab mites, trombi-culid mites, flies (stinging and sucking), parasitic fly larvae, lice, hair lice, bird lice and fleas. These parasites include:

From the order of the Anoplurida, for example, Haematopinus spp., Linognathus spp., Pediculus spp., Phtirus spp., Solenopotes spp.

From the order of the Mallophagida and the sub-orders Amblycerina and Ischnocerina, for example, Trimenopon spp., Menopon spp., Trinoton spp., Bovicola spp., Wemeckiella spp., Lepikentron spp., Damalina spp., Trichodectes spp., Felicola spp.

From the order of the Diptera and the sub-orders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopyslla spp. and Ceratophyllus spp.

From the order of the Heteropterida, for example, Cimex spp., Triatoma spp., Rhodnius spp. and Panstrongylus spp. From the order of the Blattarida, for example, Blatta orientalis, Periplaneta americana, Blattella germanica and Supella spp.

From the sub-class of the Acaria (Acarida) and the orders of the Meta- and Mesostigmata, for example, Arga ssp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds or active compound combinations according to the invention are also suitable for controlling arthropods which attack agricultural livestock, such as, for example, cattle, sheep, goats, horses, pigs, donkeys, camels, buffaloes, rabbits, chickens, turkeys, ducks, geese, honeybees, other domestic animals, such as, for example, dogs, cats, cage birds, aquarium fish, and so-called experimental animals, such as, for example, hamsters, guinea pigs, rats and mice. By combating these arthropods, it is intended to reduce deaths and decreased performances (in meat, milk, wool, hides, eggs, honey and the like), so that more economical and simpler animal keeping is made possible by using the active compounds according to the invention.

In the veterinary sector, the active compounds or active compound combinations according to the invention are used in a known manner by enteral administration, for example in the form of tablets, capsules, drinks, drenches, granules, pastes, boli, the feed-through method, suppositories, by parenteral administration, such as, for example, by means of injections (intramuscular, subcutaneous, intravenous, intraperitoneal and the like), implants, by nasal application, by dermal administration, for example in the form of dipping or bathing, spraying, pouring-on and spotting-on, washing, dusting, and with the aid of shaped articles which comprise active compound, such as collars, ear tags, tail marks, limb bands, halters, marking devices and the like.

When administered to livestock, poultry, domestic animals and the like, the active compounds or active compound combinations can be used as formulations (for example powders, emulsions, flowables) which comprise the active compounds in an amount of 1 to 80% by weight, either directly or after dilution by a factor of 100 to 10 000, or they may be used in the form of a chemical bath.

Furthermore, it has been found that the compounds or active compound combinations according to the invention have a potent insecticidal action against insects which destroy industrial materials.

The following insects may be mentioned by way of example and as being preferred, but without any limitation:

Beetles, such as

Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutus.
   Dermapterans, such as
Sirex juvencus, Urocerus gigas, Urocerus gigas taignus, Urocerus augur.
   Termites, such as
Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus.
   Bristletails, such as Lepisma saccharina.

Industrial materials are to be understood as meaning, in the present context, non-live materials, such as, preferably, synthetic materials, glues, sizes, paper and board, leather, wood and timber products, and paint.

The materials to be very particularly preferably protected against attack by insects are wood and timber products.

Wood and timber products which can be protected by the composition according to the invention or mixtures comprising such a composition are to be understood as meaning, for example:
construction timber, wooden beams, railway sleepers, bridge components, jetties, wooden vehicles, boxes, pallets, containers, telephone poles, wood cladding, windows and doors made of wood, plywood, particle board, joiner's articles, or wood products which, quite generally, are used in the construction of houses or in joinery.

The active compounds or active compound combinations can be used as such, in the form of concentrates or generally customary formulations, such as powders, granules, solutions, suspensions, emulsions or pastes.

The formulations mentioned can be prepared in a manner known per se, for example by mixing the active compounds with at least one solvent or diluent, emulsifier, dispersant and/or binder or fixative, water repellent, if appropriate desiccants and UV stabilizers and, if appropriate, colorants and pigments and other processing auxiliaries.

The insecticidal compositions or concentrates used for the protection of wood and wooden materials comprise the active compound according to the invention in a concentration of 0.0001 to 95% by weight, in particular 0.001 to 60% by weight.

The amount of the compositions or concentrates employed depends on the species and the occurrence of the insects and on the medium. The optimum rate of application can be determined upon use in each case by a test series. However, in general, it suffices to employ 0.0001 to 20% by weight, preferably 0.001 to 10% by weight, of the active compound, based on the material to be protected.

The solvent and/or diluent used is an organochemical solvent or solvent mixture and/or an oily or oil-type organochemical solvent or solvent mixture of low volatility and/or a polar organochemical solvent or solvent mixture and/or water and, if appropriate, an emulsifier and/or wetting agent.

Organochemical solvents which are preferably employed are oily or oil-type solvents having an evaporation number of above 35 and a flashpoint of above 30° C., preferably above 45° C. Substances which are used as such oily and oil-type solvents which have low volatility and are insoluble in water are suitable mineral oils or their aromatic fractions, or mineral-oil-containing solvent mixtures, preferably white spirit, petroleum and/or alkylbenzene.

Substances which are advantageously used are mineral oils with a boiling range of 170 to 220° C., white spirit with a boiling range of 170 to 220° C., spindle oil with a boiling range of 250 to 350° C., petroleum or aromatics of boiling range 160 to 280° C., essence of turpentine and the like.

In a preferred embodiment, liquid aliphatic hydrocarbons with a boiling range of 180 to 210° C. or high-boiling mixtures of aromatic and aliphatic hydrocarbons with a boiling range of 180 to 220° C. and/or spindle oil and/or monochloronaphthalene, preferably α-monochloronaphthalene, are used.

The organic oily or oil-type solvents of low volatility having an evaporation number of above 35 and a flashpoint of above 30° C., preferably above 45° C., can be partially replaced by organochemical solvents of high or medium volatility, with the proviso that the solvent mixture also has an evaporation number of above 35 and a fashpoint of above 30° C., preferably above 45° C., and that the insecticide/fungicide mixture is soluble or emulsifiable in this solvent mixture.

In a preferred embodiment, part of the organochemical solvent or solvent mixture is replaced by an aliphatic polar organochemical solvent or solvent mixture. Substances which are preferably used are aliphatic organochemical solvents having hydroxyl and/or ester and/or ether groups, such as, for example, glycol ethers, esters and the like.

The organochemical binders used within the scope of the present invention are the synthetic resins and/or binding drying oils which are known per se and can be diluted with water and/or are soluble or dispersible or emulsifiable in the organochemical solvents employed, in particular binders composed of, or comprising, an acrylate resin, a vinyl resin, for example polyvinyl acetate, polyester resin, polycondensation or polyaddition resin, polyurethane resin, alkyd resin or modified alkyd resin, phenol resin, hydrocarbon resin, such as indene/cumarone resin, silicone resin, drying vegetable and/or drying oils and/or physically drying binders based on a natural and/or synthetic resin.

The synthetic resin used as the binder can be employed in the form of an emulsion, dispersion or solution. Up to 10% by weight of bitumen or bituminous substances can also be used as binders. In addition, colorants, pigments, water repellents, odour-masking substances and inhibitors or anticorrosives known per se and the like can also be employed.

The composition or the concentrate preferably comprises, in accordance with the invention, at least one alkyd resin or modified alkyd resin and/or a drying vegetable oil as the organochemical binder. Preferably used according to the invention are alkyd resins with an oil content of over 45% by weight, preferably 50 to 68% by weight.

All or some of the abovementioned binder can be replaced by a fixative (mixture) or a plasticizer (mixture). These additives are intended to prevent volatilization of the active compounds and crystallization or precipitation. They preferably replace 0.01 to 30% of the binder (based on 100% of binder employed).

The plasticizers are from the chemical classes of the phthalic esters, such as dibutyl phthalate, dioctyl phthalate or benzyl butyl phthalate, the phosphoric esters, such as tributyl phosphate, the adipic esters, such as di-(2-ethylhexyl) adipate, the stearates, such as butyl stearate or amyl stearate, the oleates, such as butyl oleate, the glycerol ethers or relatively high-molecular-weight glycol ethers, glycerol esters and p-toluenesulphonic esters.

Fixatives are chemically based on polyvinyl alkyl ethers, such as, for example, polyvinyl methyl ether, or ketones, such as benzophenone or ethylenebenzophenone.

Particularly suitable as a solvent or diluent is also water, if appropriate as a mixture with one or more of the abovementioned organochemical solvents or diluents, emulsifiers and dispersants.

Particularly effective protection of wood is achieved by large-scale industrial impregnation processes, for example vacuum, double-vacuum or pressure processes.

If appropriate, the ready-to-use compositions can additionally comprise other insecticides and, if appropriate, additionally one or more fungicides.

Suitable additional components which may be admixed are, preferably, the insecticides and fungicides mentioned in WO 94/29 268. The compounds mentioned in that document are expressly part of the present application.

Very particularly preferred components which may be admixed are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyphenoxid and triflumuron,
and fungicides, such as epoxyconazole, hexaconazole, azaconazole, propiconazole, tebuconazole, cyproconazole, metconazole, imazalil, dichlofluanid, tolylfluanid, 3-iodo-2-propynylbutyl carbamate, N-octylisothiazolin-3-one and 4,5-dichloro-N-octylisothiazolin-3-one.

The compounds or active compound combinations according to the invention can at the same time be employed for protecting objects which come into contact with salt water or brackish water, in particular hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis(trialkyltin) sulphides, tri-n-butyltin laurate, tri-n-butyltin chloride, copper(I) oxide, triethyltin chloride, tri-n-butyl-(2-phenyl-4-chlorophenoxy)tin, tributyltin oxide, molybdenum disulphide, antimony oxide, polymeric butyl titanate, phenyl(bispyridine)bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combination with the antifouling compositions according to the invention are:

algicides such as

2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;
   fungicides such as
benzo[b]thiophenecarboxylic acid cyclohexylamide S,S-dioxide, dichlofluanid, fluorfolpet, 3-iodo-2-propynyl butylcarbamate, tolylfluanid and azoles such as
azaconazole, cyproconazole, epoxyconazole, hexaconazole, metconazole, propiconazole and tebuconazole;
molluscicides such as
fentin acetate, metaldehyde, methiocarb, niclosamid, thiodicarb and trimethacarb;
   or conventional antifouling active compounds such as
4,5-dichloro-2-octyl-4-isothiazolin-3-one, diiodomethylparatryl sulphone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-etrachloro-4-(methylsulphonyl)pyridine, 2,4,5,6-tetrachloroisophthalonitrile, tetramethylthiuram disulphide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as rosin to allow controlled release of the active compounds. Furthermore, the paints may comprise plasticizers, modifiers which affect the theological properties and other conventional constituents. The compounds according to the invention or the abovementioned mixtures may also be incorporated into self-polishing antifouling systems.

The active compounds or active compound combinations are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:

From the order of the Scorpionidea, for example, Buthus occitanus.

From the order of the Acarina, for example, Argas persicus, Argas reflexus, Bryobia ssp., Dermanyssus gattimae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalis, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

They are used in the household insecticides sector alone or in combination with other suitable active compounds such as phosphoric esters, carbamates, pyrethroids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The active compounds or active compound combinations according to the invention can also be used as defoliants, desiccants, haulm killers and, in particular, as weed killers. Weeds in the broadest sense are understood as meaning all plants which grow at locations where they are undesired. Whether the substances according to the invention act as nonselective or selective herbicides depends essentially on the application rate.

The active compounds or active compound combinations according to the invention can be used for example in the following plants:
Dicotyledonous weeds of the genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindemia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.
Dicotyledonous crops of the genera: Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia.
Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.
Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds or active compound combinations according to the invention is in no way restricted to these genera, but extends in the same manner to other plants.

Depending on the concentration, the active compounds or active compound combinations according to the invention are suitable for the nonselective weed control on, for example, industrial terrains and railway tracks and on paths and locations with and without trees. Likewise the active compounds according to the invention can be employed for controlling weeds in perennial crops, for example forests, ornamental tree plantings, orchards, vineyards, citrus groves, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The compounds or active compound combinations according to the invention have strong herbicidal activity and a broad activity spectrum when used on the soil and on aerial plant parts. To a certain extent, they are also suitable for the selective control of monocotyledonous and dicotyledonous weeds in monocotyledonous and dicotyledonous crops, both pre- and post-emergence.

At certain concentrations or application rates, the active compounds or active compound combinations according to the invention can also be employed for controlling animal pests and fungal or bacterial plant diseases. If appropriate, they can also be used as intermediates or precursors for the synthesis of other active compounds.

The active compounds or active compound combinations can be converted into the customary formulations, such as solutions, emulsions, wettable powders, suspensions, powders, dusting agents, pastes, soluble powders, granules, suspoemulsion concentrates, natural and synthetic materials impregnated with active compound, and microcapsules in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers.

If the extender used is water, it is also possible to use, for example, organic solvents as auxiliary solvents. Suitable liquid solvents are essentially: aromatics, such as xylene, toluene or alkylnaphthalenes, chlorinated aromatics and chlorinated aliphatic hydrocarbons, such as chlorobenzenes, chloroethylenes or methylene chloride, aliphatic hydrocarbons, such as cyclohexane or paraffins, for example mineral oil fractions, mineral and vegetable oils, alcohols, such as butanol or glycol, and also their ethers and esters, ketones, such as acetone, methyl ethyl ketone, methyl isobutyl ketone or cyclohexanone, strongly polar solvents, such as dimethylformamide and dimethyl sulphoxide, and also water.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates, suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; suitable emulsifiers and/or foam-formers are: for example non-ionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and protein hydrolysates; suitable dispersants are: for example lignosulphite waste liquors and methylcellulose.

Tackifiers such as carboxymethylcellulose and natural and synthetic polymers in the form of powders, granules or lattices, such as gum arabic, polyvinyl alcohol and polyvinyl acetate, and also natural phospholipids, such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalo-cyanine dyestuffs, and trace nutrients such as salts of iron, manganese, boron, copper, cobalt, molybdenum and zinc.

The formulations generally comprise between 0.1 and 95 per cent by weight of active compound, preferably between 0.5 and 90%.

The active compounds according to the invention, as such or in their formulations, can also be used for weed control purposes as a mixture with known herbicides and/or with substances which improve crop plant compatibility (“safeners”), ready mixes or tank mixes being possible. Mixtures with herbicide products which contain one or more known herbicides and a safener are hence also possible.

Herbicides which are suitable for the mixtures are known herbicides, for example
acetochlor, acifluorfen (-sodium), aclonifen, alachlor, alloxydim (-sodium), ametryne, amicarbazone, amidochlor, amidosulfuron, anilofos, asulam, atrazine, azafenidin, azimsulfuron, beflubutamid, benazolin (-ethyl), benfuresate, bensulfuron (-methyl), bentazone, benzfendizone, benzobicyclon, benzofenap, benzoylprop (-ethyl), bialaphos, bifenox, bispyribac (-sodium), bromobutide, bromofenoxim, bromoxynil, butachlor, butafenacil (-allyl), butroxydim, butylate, cafenstrole, caloxydim, carbetamide, carfentrazone (-ethyl), chlomethoxyfen, chloramben, chloridazon, chlorimuron (-ethyl), chlornitrofen, chlorsulfuron, chlortoluron, cinidon (-ethyl), cinmethylin, cinosulfuron, clefoxydim, clethodim, clodinafop (-propargyl), clomazone, clomeprop, clopyralid, clopyrasulfuron (-methyl), cloransulam (-methyl), cumyluron, cyanazine, cybutryne, cycloate, cyclosulfamuron, cycloxydim, cyhalofop (-butyl), 2,4-D, 2,4-DB, desmedipham, diallate, dicamba, dichlorprop (-P), diclofop (-methyl), diclosulam, diethatyl (-ethyl), difenzoquat, diflufenican, diflufenzopyr, dimefuron, dimepiperate, dimethachlor, dimethametryn, dimethenamid, dimexyflam, dinitramine, diphenamid, diquat, dithiopyr, diuron, dymron, epropodan, EPTC, esprocarb, ethalfluralin, ethametsulfuron (-methyl), ethofumesate, ethoxyfen, ethoxysulfuron, etobenzanid, fenoxaprop (-P-ethyl), fentrazamide, flamprop (-isopropyl, -isopropyl-L, -methyl), flazasulfuron, florasulam, fluazifop (-P-butyl), fluazolate, flucarbazone (-sodium), flufenacet, flumetsulam, flumiclorac (-pentyl), flumioxazin, flumipropyn, flumetsulam, fluometuron, fluorochloridone, fluoroglycofen (-ethyl), flupoxam, flupropacil, flurpyrsulfuron (-methyl, -sodium), flurenol (-butyl), fluridone, fluroxypyr (-butoxypropyl, -meptyl), flurprimidol, flurtamone, fluthiacet (-methyl), fluthiamide, fomesafen, foramsulfuron, glufosinate (-ammonium), glyphosate (-isopropylammonium), halosafen, haloxyfop (-ethoxyethyl, -P-methyl), hexazinone, imazamethabenz (-methyl), imazamethapyr, imazamox, imazapic, imazapyr, imazaquin, imazethapyr, imazosulfuron, iodosulfuron (-methyl, -sodium), ioxynil, isopropalin, isoproturon, isouron, isoxaben, isoxachlortole, isoxaflutole, isoxapyrifop, lactofen, lenacil, linuron, MCPA, mecoprop, mefenacet, mesosulfurone, mesotrione, metamitron, metazachlor, methabenzthiazuron, metobenzuron, metobromuron, (alpha-) metolachlor, metosulam, metoxuron, metribuzin, metsulfuron (-methyl), molinate, monolinuron, naproanilide, napropamide, neburon, nicosulfuron, norflurazon, orbencarb, oryzalin, oxadiargyl, oxadiazon, oxasulfuron, oxaziclomefone, oxyfluorfen, paraquat, pelargonic acid, pendimethalin, pendralin, pentoxazone, phenmedipham, picolinafen, pinoxaden, piperophos, pretilachlor, primisulfuron (-methyl), profluazol, prometryn, propachlor, propanil, propaquizafop, propisochlor, propoxycarbazone (-sodium), propyzamide, prosulfocarb, prosulfuron, pyraflufen (-ethyl), pyrazogyl, pyrazolate, pyrazosulfuron (-ethyl), pyrazoxyfen, pyribenzoxim, pyributicarb, pyridate, pyridatol, pyriftalide, pyriminobac (-methyl), pyrithiobac (-sodium), quinchlorac, quinmerac, quinoclamine, quizalofop (-P-ethyl, -P-tefuryl), rimsulfuron, sethoxydim, simazine, simetryn, sulcotrione, sulfentrazone, sulfometuron (-methyl), sulfosate, sulfosulfuron, tebutam, tebuthiuron, tepraloxydim, terbuthylazine, terbutryn, thenylchlor, thiafluamide, thiazopyr, thidiazimin, thifensulfuron (-methyl), thiobencarb, tiocarbazil, tralkoxydim, triallate, triasulfuron, tribenuron (-methyl), triclopyr, tridiphane, trifluralin, trifloxysulfuron, triflusulfuron (-methyl), tritosulfuron.

A mixture with other known active compounds, such as fungicides, insectides, acaricides, nematicides, bird repellents, plant nutrients and soil conditioners, is also possible.

The active compounds or active compound combinations can be applied as such, in the form of their formulations or the use forms prepared therefrom by further dilution, such as ready-to-use solutions, suspensions, emulsions, powders, pastes and granules. They are applied in the customary manner, for example by pouring, spraying, atomizing, spreading.

The active compounds or active compound combinations according to the invention can be applied both before and after plant emergence. They can also be incorporated into the soil prior to sowing.

The application rate of active compound can vary within a substantial range. Essentially, it depends on the nature of the desired effect. In general, the application rates are between 1 g and 10 kg of active compound per hectare of soil area, preferably between 5 g and 5 kg per ha.

The advantageous effect of the compatibility with crop plants of the active compound combinations according to the invention is particularly pronounced at certain concentration ratios. However, the weight ratios of the active compounds in the active compound combinations can be varied within relatively wide ranges. In general, from 0.001 to 1000 parts by weight, preferably from 0.01 to 100 parts by weight, particularly preferably 0.05 to 20 parts by weight, of one of the compounds which improves crop plant compatibility (antidotes/safeners) mentioned above under (c′) are present per part by weight of active compound of the formula (I).

The active compound combinations according to the invention are generally applied in the form of finished formulations. However, the active compounds contained in the active compound combinations can, as individual formulations, also be mixed during use, i.e. be applied in the form of tank mixes.

For certain applications, in particular by the post-emergence method, it may furthermore be advantageous to include, as further additives in the formulations, mineral or vegetable oils which are compatible with plants (for example the commercial preparation “Rako Binol”), or ammonium salts, such as, for example, ammonium sulphate or ammonium thiocyanate.

The novel active compound combinations can be used as such, in the form of their formulations or the use forms prepared therefrom by further dilution, such as ready-to-use solutions, suspensions, emulsions, powders, pastes and granules. Application is in the customary manner, for example by watering, spraying, atomizing, dusting or scattering.

The application rates of the active compound combinations according to the invention can be varied within a certain range; they depend, inter alia, on the weather and on soil factors. In general, the application rates are between 0.001 and 5 kg per ha, preferably between 0.005 and 2 kg per ha, particularly preferably between 0.01 and 0.5 kg per ha.

The active compound combinations according to the invention can be applied before and after emergence of the plants, that is to say by the pre-emergence and post-emergence method.

Depending on their properties, the safeners to be used according to the invention can be used for pretreating the seed of the crop plant (seed dressing) or can be introduced into the seed furrows prior to sowing or be used separately prior to the herbicide or together with the herbicide, before or after emergence of the plants.

Preparation and use of the active compounds according to the invention are illustrated in the examples below.

### PREPARATION EXAMPLES

### Example I-a-1

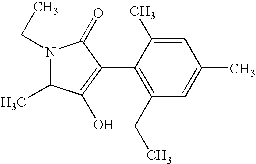

2-Ethyl-4,6-dimethylphenylacetic acid (1.5 g, 7.8 mmol), 1.36 g of ethyl 2-ethylaminopropionate (1.2 eq) and 4.3 ml of triethylamine (3.9 eq) were initially charged in 60 ml of tetrahydrofuran (THF) and stirred at room temperature for 10 min. 0.44 ml of phosphorus oxychloride was added, and the mixture was heated under reflux to the boil for 6 h. The solution was concentrated under reduced pressure, the residue was then taken up in the ethyl acetate and the residue formed was filtered off. After drying of the organic phase with sodium sulphate and removal of the solvent under reduced pressure, the resulting product A was reacted without further purification.

To this end, 1.22 g of potassium tert-butoxide was initially charged in a solution of 18 ml of DMF, and A, in a solution of 35 ml of DMF, was added dropwise over a period of one hour. The mixture was stirred at room temperature for 6 h and then poured into water. The solution was adjusted to pH=2 using hydrochloric acid and stirred for 20 min. The precipitate formed was filtered off, washed with n-heptane and dried under reduced pressure. Yield: 630 mg (2.3 mmol), 29.5% of theory, m.p. 205-207° C.

The following compounds of the formula (I-a) are obtained analogously to example (I-a-1) and in accordance with the general statements on the preparation:

(I-a)

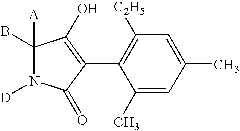

| Ex. No. | B | A | D | m.p. ° C. |
|---|---|---|---|---|
| I-a-2 | H | C₂H₅ | CH₃ | 231-234 |
| I-a-3 | H | H | 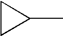 | 220-223 |

| | | | |
|---|---|---|---|
| I-a-4 | H | 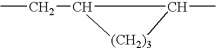 | 230 |

### Example I-b-1

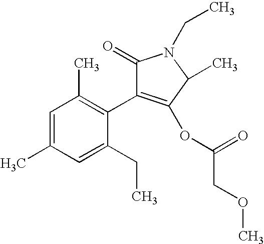

0.137 g of the compound of Example I-a-1 was initially charged in 9 ml of anhydrous ethyl acetate, and 0.08 ml of triethylamine was added dropwise. Under reflux, 0.05 ml of methoxyacetyl chloride in 3 ml of ethyl acetate was added dropwise, and the mixture was stirred under reflux for 4 h. After cooling, the mixture was washed with 10 ml of sodium bicarbonate solution, the aqueous phase was reextracted with ethyl acetate and the organic phases were combined, dried over magnesium sulphate and concentrated using a rotary evaporator.

Yield: 0.144 g, 79.2% of theory, ¹H-NMR, 300 MHz, CDCl₃: δ=2.13, 2.28 (2s, Ar CH₃), 3.32 (s, OCH₃) ppm.

The following compounds of the formula (I-b) are obtained analogously to Example (I-b-1) and in accordance with the general statements on the preparation:

| | |
|---|---|
| | (I-b) |
| 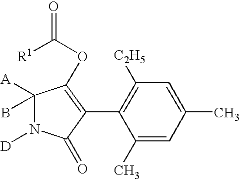 | |

| Ex. No. | B | A | D | R¹ | m. p. ° C. |
|---|---|---|---|---|---|
| I-b-2 | CH₃ | CH₃ | H | i-C₃H₇ | 122 |
| I-b-3 | CH₃ | i-C₃H₇ | H | t-C₄H₉ | *1.1 (t, 3H, Ar—CH₂—CH₃), 2.37 |
| | | | | | (m, 2H, H₃C—CH₂—Ar) |
| I-b-4 | H | i-C₃H₇ | H | t-C₄H₉ | 116 |
| I-b-5 | H | i-C₄H₉ | H | H₃CO—CH₂— | *0.95 (m, 6H, CH(CH₃)₂) |
| | | | | | 2.3 (s, 3H, Ar-4-CH₃) |
| I-b-6 | H | i-C₄H₉ | H | i-C₃H₇ | *1.80 (m, 1H, CH₂—CH(CH₃)₂) |
| | | | | | 2.10, 2.15 (2s, 3H, Ar-2-CH₃) |
| | | | | | 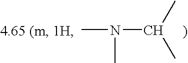 |
| I-b-7 | H | i-C₃H₇ | H | i-C₃H₇ | 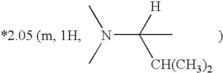 |
| | | | | | 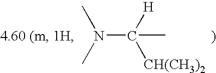 |
| I-b-8 | H | i-C₄H₉ | H | t-C₄H₉ | 67 |
| I-b-9 | CH₃ | CH₃ | H | CH₃ | 184-185 |
| I-b-10 | CH₃ | 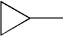 | H | i-C₃H₇ | 124-130 |
| I-b-11 | CH₃ | C₂H₅ | H | i-C₃H₇ | 133-135 |
| I-b-12 | CH₃ | CH₃ | H | H₃CO—CH₂— | 137 |
| I-b-13 | CH₃ | i-C₄H₉ | H | H₃CO—CH₂— | *1.40, 1.45 |
| | | | | | 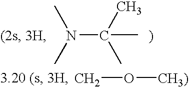 |
| I-b-14 | CH₃ | i-C₄H₉ | H | t-C₄H₉ | 144 |
| I-b-15 | CH₃ | i-C₃H₇ | H | i-C₃H₇ | 148 |
| I-b-16 | CH₃ | CH₃ | H | H₅C₂O—CH₂— | 107-109 |
| I-b-17 | CH₃ | C₂H₅ | H | H₅C₂O—CH₂— | *2.49 (m, 2H, CH₂—Ar) |
| | | | | | 4.01 (dq, 2H, CO—CH₂O) |
| I-b-18 | C₂H₅ | C₂H₅ | H | i-C₃H₇ | 145-147 |

*¹H-NMR, 300 MHz, CDCl₃: δ = ppm.

### Example I-c-1

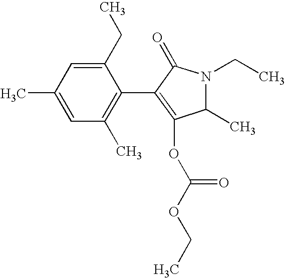

0.137 g (0.5 mmol) of the compound of Example I-a-1 and 0.056 g (0.55 mmol) of triethylamine in 8 ml of anhydrous dichloromethane are stirred at room temperature for 15 minutes, and 0.08 g (0.55 mmol) of ethyl chloroformate is then added. The mixture is stirred and monitored by thin-layer chromatography. After cooling, 5 ml of sodium carbonate solution are added, and the organic phase is then separated off. The solvent is distilled off and the residue is purified by silica gel column chromatography (ethyl acetate/n-heptane, 1:1).

Yield: 116.1 mg, 44.8% of theory. (¹H-NMR, CDCl₃, 2.43 ppm (m, CH₂-Aryl), 1.11 ppm CH₃—CH₂-aryl, 6.86 ppm (s broad 2H aryl-H).

The following compounds of the formula (I-c) are obtained analogously to Example (I-c-1) and in accordance with the general statements on the preparation:

| | |
|---|---|
| | (I-c) |
| 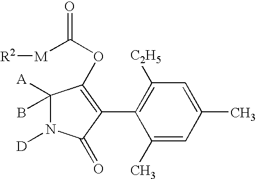 | |

| Ex. No. | B | A | D | M | R² | m. p. ° C. |
|---|---|---|---|---|---|---|
| I-c-2 | CH₃ | CH₃ | H | O | C₂H₅ | 83 |
| I-c-3 | CH₃ | i-C₃H₇ | H | O | C₂H₅ | Resin |
| I-c-4 | H | H | 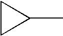 | O | C₂H₅ | *2.26 (s, 3H; 4-CH₃—Ar), 4.32 (s, 2H; —NCH₂—), 4.21 (q, 2H, O—CH₂CH₃) |
| I-c-5 | H | 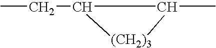 | | O | C₂H₅ | *2.29 (s, 3H, 4-CH₃—Ar), 4.17 (m, 2H, O—CH₂—CH₃) |
| I-c-6 | CH₃ | C₃H₇ | H | O | C₂H₅ | *2.26 (s, 3H, 4-CH₃—Ar), 1.45, (d, 3H |
| | | | | | | —N—C—CH₃), 3.98 (m, 2H, O—CH₂—CH₃) |
| I-c-7 | CH₃ | 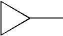 | H | O | C₂H₅ | *2.26 (s, 3H, 4-CH₃—Ar), 1.52 (d, 3H, —N—C—CH₃), 4.01 (q, 2H, O—CH₂—CH₃) |
| I-c-8 | CH₃ | C₂H₅ | H | O | C₂H₅ | 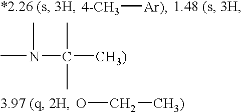 |
| I-c-9 | C₂H₅ | C₂H₅ | H | O | C₂H₅ | 117-121 |
| I-c-10 | H | C₂H₅ | CH₃ | O | C₂H₅ | *2.16 (s, 3H, Ar—CH₃) |
| | | | | | | 4.17 (m, 2H, OCH₂CH₃) |
| I-c-11 | CH₃ | i-C₄H₉ | H | O | C₂H₅ | 118 |

*¹H-NMR (300 MHz in CDCl₃), shifts δ in ppm.

### Example I-d-1

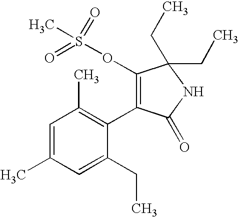

0.076 g (0.265 mmol) of the compound of Ex. I-a′-5 is initially charged in 10 ml of dichloromethane, and 0.04 ml of triethylamine is added. 0.02 ml of methylsulfonide chloride is added a little at a time, and the mixture is stirred at room temperature for 24 h. 5% strength NaHCO₃ solution is added, the organic phase is separated off and the aqueous phase is extracted with dichloromethane. The combined organic phases are dried with sodium sulphate and then, under reduced pressure, evaporated to dryness, and the residue formed is purified chromatographically using ethyl acetate/n-heptane (gradient 1/4 to 2/1).

Yield: 0.048 g (47% of theory), m.p. 187-190° C.

### Example I-d-2

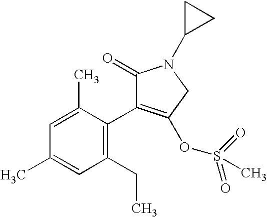

Example 1-d-2 of melting point 102° C. is obtained analogously to Ex. I-d-1.

Examples (I-a′) which may be mentioned are the following compounds:

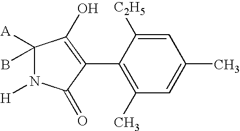

| Ex. No. | A | B | m. p. ° C. |
|---|---|---|---|
| I-a′-1 | CH₃ | CH₃ | >220° C. |
| I-a′-2 | i-C₃H₇ | CH₃ | *2.00 (m, 1H, CH(CH₃)₂, 6.90 |
| | | | (s, br, 2H, Ar H) |
| I-a′-3 | i-C₃H₇ | H | *2.20 (m, 1H, CH(CH₃)₂, 3.95 |
| | | | (m, 1H, CH—CH(CH₃)₂) |
| I-a′-4 | i-C₄H₉ | H | *0.95 (2d, 6H, CH(CH₃)₂, 4.00 |
| | | | (d, br, 1H, CH-i-C₄H₉) |
| I-a′-5 | C₂H₅ | C₂H₅ | 182-183 |
| I-a′-6 | C₃H₇ | CH₃ | 203 |
| I-a′-7 | 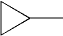 | CH₃ | 132 |
| I-a′-8 | C₂H₅ | CH₃ | 131 |
| I-a′-9 | i-C₄H₉ | CH₃ | 80 |

*¹H-NMR (300 MHz in CDCl₃), shifts δ in ppm.

### Example II-1

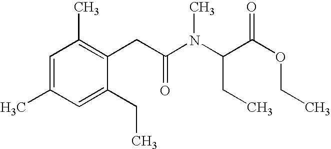

1.5 g of 2-ethyl-4,6-dimethylphenylacetic acid (7.8 mmol) and 1.4 g of 2-ethyl 2-ethylsarcosine hydrochloride and 4.24 ml of triethylamine are initially charged in 60 ml of THF and stirred at room temperature for 10 min. 0.44 ml of phosphorus oxychloride is added, and the mixture is then heated under reflux for 6 h. The reaction solution is concentrated under reduced pressure and taken up in ethyl acetate, and the insoluble residue is separated off. The organic solution is washed with water and then dried with sodium sulphate and concentrated. After filtration over silica gel using ethyl acetate/n-heptane (1/4) the 1.68 g obtained are directly reacted to prepare Example I-a-2.

Further intermediates of the formula (II) were prepared analogously to Example (II-1) and in accordance with the general statements on the preparation and, without further characterization, used directly for preparing compounds of the formula (I-a).

### Preparation of 2-ethyl-4,6-dimethylphenylacetic Acid (XVIII.1)

Component K

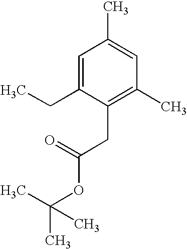

At 18° C., 2.998 g of tert-butyl acetate are added to 30.5 mmol of a freshly prepared lithium dicyclohexylamide solution. During the addition, the temperature increases from 18 to 31° C. After 5 min, at 28° C., a degassed mixture of 5 g of 2-ethyl-4,6-dimethylbromobenzene and 135 mg of bis(dibenzylideneacetone)palladium and 0.047 g of tri-tert-butylphosphine, as a 0.5 molar solution in toluene, is added. The temperature increases to 54° C., and the mixture is then cooled to room temperature. The mixture is poured into a mixture of 30 ml of hydrochloric acid and 200 ml of water and extracted twice with 600 ml of dichloromethane. The organic phase is separated off and dried using sodium sulphate. The 5.7 g of an viscous oil are reacted further without further purification.

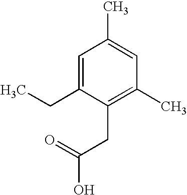

At room temperature 5.7 g of component K are stirred in 100 ml of formic acid for 2 h. The mixture is then poured into 500 ml of ice-water and stirred at room temperature for 30 min, and the solid formed is filtered off with suction. For drying, the residue is taken up in dichloromethane and this solution is dried with sodium phosphate. In this manner, 3.67 g of 2-ethyl-4,6-dimethylphenylacetic acid are obtained after concentration, which corresponds to a total yield of 81% over all steps. M.p. 128-129° C.

### USE EXAMPLES

### Example A

Herbicidal Post-emergence Action

Seeds of monocotyledonous and dicotyledonous weed and crop plants are placed into sandy loam in wood fibre pots, covered with soil and cultivated in a greenhouse under good growth conditions. 2-3 weeks after sowing, the test plants are treated at the one-leaf stage. The test compounds, formulated as wettable powders (WP), are, in various dosages with a water application rate of 600 l/ha (converted), with 0.2% of wetting agent added, sprayed onto the green parts of the plants. After the test plants were kept in the greenhouse under optimum growth conditions for about 3 weeks, the effect of the preparations is rated visually in comparison to untreated controls (herbicidal effect in per cent (%): 100% effect=the plants have died, 0% effect=like control plants).

| Greenhouse | g of a.i/ha | Avena sativa | Lolium | Setaria |
|---|---|---|---|---|
| Ex. I-c-3 | 320 | 90 | 100 | 90 |
| Ex. I-b-3 | 320 | 90 | 70 | 80 |
| Ex. I-a-1 | 320 | 80 | 100 | 90 |
| Ex. Ia-2 | 320 | 80 | 100 | 90 |
| Ex.-I-c-1 | 320 | 90 | 100 | 100 |

| Green- | | Avena | | | | |
|---|---|---|---|---|---|---|
| house | G of a.i./ha | sativa | Lolium | Setaria | Sinapis | Stellaria |
| Ex. I-b-2 | 320 | 100 | 100 | 100 | 70 | 80 |
| Ex. I-c-2 | 320 | 100 | 100 | 100 | 70 | 70 |
| Ex. I-a-3 | 320 | 80 | 100 | 90 | 70 | — |

### Example B

Herbicidal Post-emergence Action

Seeds of monocotyledonous and dicotyledonous weed and crop plants are placed into sandy loam in wood fibre pots or in plastic pots, covered with soil and cultivated in a greenhouse, during the vegetation period also outdoors outside of the greenhouse, under good growth conditions. 2-3 weeks after sowing, the test plants are treated at the one- to three-leaf stage. The test compounds, formulated as wettable powders (WP) or emulsifiable concentrates (EC) are, in various dosages with a water application rate of 300 l/ha (converted), with wetting agent (0.2 to 0.3%) added, sprayed onto the plants and the surface of the soil. 3-4 weeks after the treatment of the test plants, the effect of the preparations is rated visually in comparison to untreated controls (herbicidal effect in per cent (%): 100% effect=the plants have died, 0% effect=like control plants).

Use of Safeners

If it is additionally to be tested as to whether safeners can improve the plant compatibility of test substances in the case of crop plants, the following options are used for applying the safener:
seeds of the crop plants are, before sowing, dressed with the safener substance (the amount of safener is stated in per cent, based on the weight of the seed)
before the application of the test substances, the crop plants are sprayed with the safener at a certain application rate per hectare (usually 1 day before the application of the test substance)
the safener is applied together with the test substance as a tank mix (the amount of safener is stated in g/ha or as a ratio, based on the herbicide).

By comparing the effect of the test substances on crop plants without or with safener treatment, it is possible to assess the effect of the safener substance.

Vessel Trials with Cereals in the Greenhouse

Mefenpyr applied 1 day prior to the application of herbicide

| | Application rate | Summer barley | Summer wheat |
|---|---|---|---|
| | g of a.i./ha | observed (%) | observed (%) |
| Example | 100 | 20 | 25 |
| I-a′-2 | | | |
| Example | 100 + 100 | 10 | 5 |
| I-a′-2 + | | | |
| mefenpyr | | | |

| | Application rate | Summer barley | Summer wheat |
|---|---|---|---|
| | g of a.i./ha | observed (%) | observed (%) |
| Example I-c-3 | 100 | 30 | 20 |
| | 50 | 30 | 15 |
| | 25 | 10 | 10 |
| Example I-c-3 + | 100 + 100 | 10 | 10 |
| mefenpyr | 50 + 100 | 10 | 10 |
| | 25 + 100 | 5 | 5 |

| | Application rate | Summer barley |
|---|---|---|
| | g of a.i./ha | observed (%) |
| Example I-b-2 | 100 | 30 |
| | 50 | 20 |
| | 25 | 15 |
| Example I-b-2 + | 100 + 100 | 15 |
| mefenpyr | 50 + 100 | 15 |
| | 25 + 100 | 10 |

| | Application rate | Summer barley |
|---|---|---|
| | g of a.i./ha | observed (%) |
| Example I-a′-6 | 100 | 30 |
| | 50 | 10 |
| Example I-a′-6 + | 100 + 100 | 10 |
| mefenpyr | 50 + 100 | 0 |

| | Application rate | Summer barley |
|---|---|---|
| | g of a.i./ha | observed (%) |
| Example I-a′-6 | 100 | 70 |
| | 50 | 50 |
| | 25 | 30 |
| | 12.5 | 10 |
| Example I-a′-6 + | 100 + 100 | 30 |
| mefenpyr | 50 + 100 | 20 |
| | 25 + 100 | 10 |
| | 12.5 + 100 | 0 |

### Example C

Phaedon Test (Spray Treatment)

| | |
|---|---|
| Solvents | 78 parts by weight of acetone |
| | 1.5 parts by weight of dimethylformamide |
| Emulsifier | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Discs of Chinese cabbage (Brassica pekinensis) are sprayed with a preparation of active compound of the desired concentration and, after they have dried, populated with larvae of the mustard beetle (Phaedon cochleariae).

After the desired period of time, the activity in % is determined. 100% means that all beetle larvae have been killed; 0% means that none of the beetle larvae have been killed.

In this test, for example, the following compound of the Preparation Examples shows good activity:

TABLE C
Plant-damaging insects
Phaedon Test (spray treatment)

| | Concentration of active compound | Kill rate |
|---|---|---|
| Active compound | in g/ha | in % after 5 d |
| Example I-b-18 | 500 | 100 |
| Example I-b-10 | 500 | 100 |

### Example D

Tetranychus-Test (Spray Treatment)

| | |
|---|---|
| Solvents | 78 parts by weight of acetone |
| | 1.5 parts by weight of dimethylformamide |
| Emulsifier | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Discs of bean leaves (Phaseolus vulgaris) which are infested by all stages of the greenhouse red spider mite (Tetranychus urticae) are sprayed with a preparation of active compound of the desired concentration.

After the desired period of time, the activity in % is determined. 100% means that all spider mites have been killed; 0% means that none of the spider mites have been killed.

In this test, for example, the following compounds of the Preparation Examples show good activity:

TABLE D
Plant-damaging mites
Tetranychus Test (OP-resistant/spray treatment)

| | Concentration of active | Kill rate in % |
|---|---|---|
| Active compound | compound in g/ha | after 5 d |
| Example I-b-18 | 100 | 100 |
| Example I-b-10 | 100 | 90 |

### Example E

Critical Concentration Test/soil Insects-treatment of Transgenic Plants

| | |
|---|---|
| Test insect: | Diabrotica balteata-larvae in soil |
| Solvent: | 7 parts by weight of acetone |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

The preparation of active compound is poured onto the soil. Here the concentration of active compound in the preparation is virtually immaterial, only the amount by weight of active compound per unit volume of soil, which is stated in ppm (mg/l), matters. The soil is filled into 0.25 l pots, and they are allowed to stand at 20° C.

Immediately after the preparation, 5 pregerminated maize corns of the cultivar YIELD GUARD (trade mark of Monsanto Comp., USA) are placed into each pot. After 2 days, the appropriate test insects are placed into the treated soil. After a further 7 days, the efficacy of the active compound is determined by counting the maize plants that have emerged (1 plant=20% activity).

### Example F

Heliothis virescens Test—Treatment of Transgenic Plants

| | |
|---|---|
| Solvent: | 7 parts by weight of acetone |
| Emulsifier: | 1 part by weight of alkylaryl polyglycol ether |

To produce a suitable preparation of active compound, 1 part by weight of active compound is mixed with the stated amount of solvent and the stated amount of emulsifier, and the concentrate is diluted with water to the desired concentration.

Soya bean shoots (Glycine max) of the cultivar Roundup Ready (trade mark of Monsanto Comp. USA) are treated by being dipped into the preparation of active compound of the desired concentration and are populated with the tobacco bud worm Heliothis virescens while the leaves are still moist.

After the desired period of time, the kill of the insects is determined.

1. A compound of the formula (I) 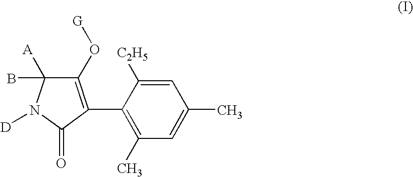
in which
G represents hydrogen (a), 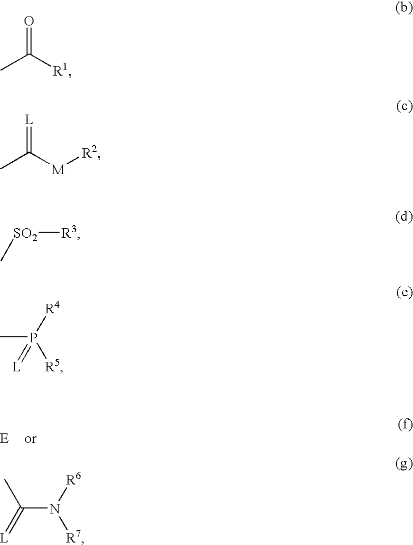
in which
E represents a metal ion equivalent or an ammonium ion,
L represents oxygen or sulphur,
M represents oxygen or sulphur,
R¹ represents in each case optionally substituted alkyl, alkenyl, alkoxyalkyl, alkylthioalkyl or polyalkoxyalkyl, or in each case optionally halogen-, alkyl-, or alkoxy-substituted cycloalkyl or heterocyclyl, or in each case optionally substituted phenyl, phenylalkyl, phenylalkenyl or heteroaryl,
R² represents in each case optionally halogen-substituted alkyl, alkenyl, alkoxyalkyl or polyalkoxyalkyl or in each case optionally substituted cycloalkyl, phenyl or benzyl,
R³, R⁴ and R⁵ independently of one another represent optionally halogen-substituted alkyl, alkoxy, alkylamino, dialkylamino, alkylthio, alkenylthio or cycloalkylthio or in each case optionally substituted phenyl, benzyl, phenoxy or phenylthio,
R⁶ and R⁷ independently of one another represent hydrogen, in each case optionally halogen-substituted alkyl, cycloalkyl, alkenyl, alkoxy, or alkoxyalkyl, in each case optionally substituted phenyl or benzyl or R⁶ and R⁷ together with the N atom to which they are attached form an optionally substituted cycle which optionally contains oxygen or sulphur,
A represents hydrogen, in each case optionally halogen-substituted alkyl, alkenyl, alkoxyalkyl or alkylthioalkyl or optionally substituted cycloalkyl,
B represents hydrogen, alkyl or alkoxyalkyl,
D represents hydrogen or an optionally substituted radical selected from the group consisting of alkyl, alkenyl, alkynyl, alkoxyalkyl, alkylthioalkyl, and cycloalkyl, or
A and D together with the atoms to which they are attached form a saturated or unsaturated cycle which optionally contains at least one heteroatom in the A,D moiety and which is unsubstituted or substituted in the A,D moiety,
provided that when
G represents hydrogen (a), then
A represents hydrogen or alkyl,
B represents hydrogen or alkyl,
D represents an optionally substituted radical selected from the group consisting of alkyl, alkenyl, alkynyl, alkoxyalkyl, alkylthioalkyl, and cycloalkyl, or
A and D together with the atoms to which they are attached form a saturated or unsaturated cycle which optionally contains at least one heteroatom in the A,D moiety and which is unsubstituted or substituted in the A,D moiety.

2. The compound according to claim 1, provided that when
G represents hydrogen (a), then
A represents hydrogen or C₁-C₈-alkyl,
B represents hydrogen or C₁-C₆-alkyl,
D represents C₁-C₈-alkyl, C₁-C₈-alkenyl, C₁-C₆-alkoxy-C₂-C₄-alkyl or C₁-C₆-alkylthio-C₂-C₄-alkyl, each of which is optionally mono- to pentasubstituted by halogen, or C₃-C₈-cycloalkyl optionally substituted with one, two or three substituents selected from the group consisting of halogen, C₁-C₄-alkyl, C₁-C₄-alkoxy and C₁-C₂-haloalkyl, or
A and D together represent a C₃-C₆-alkanediyl or C₃-C₆-alkenediyl group, wherein optionally one methylene group is replaced by oxygen or sulphur and wherein said C₃-C₆-alkanediyl or C₃-C₆-alkenediyl group is optionally substituted with one or two substituents selected from the group consisting of halogen, hydroxyl, C₁-C₄-alkyl and C₁-C₄-alkoxy, or wherein a C₃-C₆-alkanediyl, C₃-C₆-alkenediyl or C₄-C₆-alkanedienediyl group is optionally attached to two adjacent carbon atoms of said C₃-C₆-alkanediyl or C₃-C₆-alkenediyl group forming a fused ring system, or
provided that when
G represents one of the groups 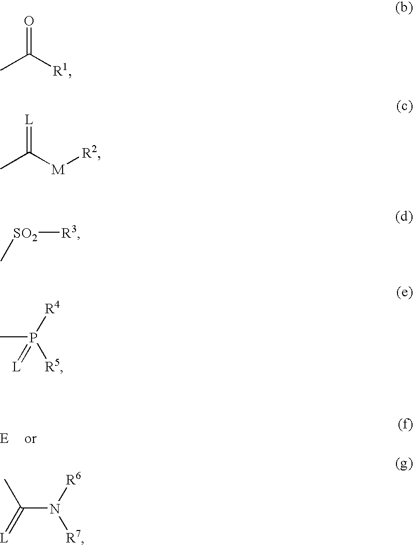
in which
E represents a metal ion equivalent or an ammonium ion,
L represents oxygen or sulphur,
M represents oxygen or sulphur,
R¹ represents C₁-C₂₀-alkyl, C₂-C₂₀-alkenyl, C₁-C₆-alkoxy-C₁-C₆-alkyl, C₁-C₆-alkylthio-C₁-C₆-alkyl or poly-C₁-C₄-alkoxy-C₁-C₄-alkyl, each of which is optionally mono- to heptasubstituted by halogen, mono- or disubstituted by cyano, monosubstituted by COR¹³, C═N—OR¹³, CO₂R¹³ or 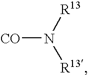
C₃-C₈-cycloalkyl optionally substituted with one, two or three substituents selected from the group consisting of halogen, C₁-C₄-alkyl and C₁-C₄-alkoxy, wherein one or two not directly adjacent methylene groups of said C₃-C₈-cycloalkyl are optionally replaced by oxygen or sulphur,
phenyl, phenyl-C₁-C₂-alkyl or phenyl-C₂-alkenyl, each of which is optionally substituted with one, two or three substituents selected from the group consisting of halogen, cyano, nitro, C₁-C₆-alkyl, C₁-C₆-alkoxy, C₁-C₆-haloalkyl, C₁-C₆-haloalkoxy, C₁-C₆-alkylthio, C₁-C₆-alkylsulfinyl and C₁-C₆-alkylsulfonyl, or
5- or 6-membered heteroaryl optionally substituted with one or two substituents selected from the group consisting of halogen and C₁-C₆-alkyl and contains one or two heteroatoms selected from the group consisting of oxygen, sulphur and nitrogen,
R¹ represents C₁-C₂₀-alkyl, C₂-C₂₀-alkenyl, C₁-C₆-alkoxy-C₂-C₆-alkyl or poly-C₁-C₆-alkoxy-C₂-C₆-alkyl, each of which is optionally mono- to trisubstituted by halogen,
C₃-C₈-cycloalkyl which is optionally substituted with one or two substituents selected from the group consisting of halogen, C₁-C₆-alkyl and C₁-C₆-alkoxy, or
phenyl or benzyl, each of which is optionally substituted with one, two or three substituents selected from the group consisting of halogen, cyano, nitro, C₁-C₆-alkyl, C₁-C₆-alkoxy, C₁-C₆-haloalkyl and C₁-C₆-haloalkoxy,
R³ represents C₁-C₈-alkyl which is optionally mono- or polysubstituted by halogen, or phenyl or benzyl, each of which is optionally substituted with one or two substituents selected from the group consisting of halogen, C₁-C₆-alkyl, C₁-C₆-alkoxy, C₁-C₄-haloalkyl, C₁-C₄-haloalkoxy, cyano and nitro,
R⁴ and R⁵ independently of one another represent C₁-C₈-alkyl, C₁-C₈-alkoxy, C₁-C₈-alkylamino, di(C₁-C₈-alkyl)amino, C₁-C₈-alkylthio or C₂-C₈-alkenylthio, each of which is optionally mono- to trisubstituted by halogen, or phenyl, phenoxy or phenylthio, each of which is optionally substituted with one, two or three substituents selected from the group consisting of halogen, nitro, cyano, C₁-C₄-alkoxy, C₁-C₄-haloalkoxy, C₁-C₄-alkylthio, C₁-C₄-haloalkylthio, C₁-C₄-alkyl and C₁-C₄-haloalkyl,
R⁶ and R⁷ independently of one another represent hydrogen, C₁-C₈-alkyl, C₃-C₈-cycloalkyl, C₁-C₈-alkoxy, C₃-C₈-alkenyl or C₁-C₈-alkoxy-C₂-C₈-alkyl, wherein said C₁-C₈-alkyl, C₃-C₈-cycloalkyl, C₁-C₈-alkoxy, C₃-C₈-alkenyl or C₁-C₈-alkoxy-C₂-C₈-alkyl is optionally mono- to trisubstituted by halogen, or phenyl or benzyl, each of which is optionally substituted with one, two or three substituents selected from the group consisting of halogen, C₁-C₈-alkyl, C₁-C₈-haloalkyl and C₁-C₈-alkoxy or R⁶ and R⁷ together represent a C₃-C₆-alkylene radical which is optionally mono- or disubstituted by C₁-C₄-alkyl and in which optionally one methylene group is replaced by oxygen or sulphur,
R¹³ represents C₁-C₆-alkyl, C₃-C₆-alkenyl, C₃-C₆-alkynyl or C₁-C₄-alkoxy-C₂-C₄-alkyl, each of which is optionally mono- to trisubstituted by halogen, or C₃-C₆-cycloalkyl which is optionally substituted with one or two substituents selected from the group consisting of halogen, C₁-C₂-alkyl and C₁-C₂-alkoxy and in which one or two not directly adjacent methylene groups are optionally replaced by oxygen, and
R^{13′} represents hydrogen, C₁-C₆-alkyl or C₃-C₆-alkenyl, then
A represents hydrogen, C₁-C₈-alkyl, C₂-C₈-alkenyl, C₁-C₆-alkoxy-C₁-C₄-alkyl or C₁-C₆-alkylthio-C₁-C₄-alkyl, wherein said C₁-C₈-alkyl, C₂-C₈-alkenyl, C₁-C₆-alkoxy-C₁-C₄-alkyl or C₁-C₆-alkylthio-C₁-C₄-alkyl is optionally mono- to trisubstituted by halogen, or C₃-C₈-cycloalkyl optionally substituted with one, two or three substituents selected from the group consisting of halogen, C₁-C₆-alkyl and C₁-C₆-alkoxy,
B represents hydrogen, C₁-C₆-alkyl or C₁-C₄-alkoxy-C₁-C₂-alkyl,
D represents hydrogen, C₁-C₈-alkyl, C₁-C₈-alkenyl, C₁-C₆-alkoxy-C₂-C₄-alkyl or C₁-C₆-alkylthio-C₂-C₄-alkyl, wherein said C₁-C₈-alkyl, C₁-C₈-alkenyl, C₁-C₆-alkoxy-C₂-₄-alkyl or C₁-C₆-alkylthio-C₂-C₄-alkyl is optionally mono- to trisubstituted by halogen, or C₃-C₈-cycloalkyl optionally substituted with one, two or three substituents selected from the group consisting of halogen, C₁-C₄-alkyl, C₁-C₄-alkoxy and C₁-C₂-haloalkyl, or
A and D together represent a C₃-C₆-alkanediyl or C₃-C₆-alkenediyl group, wherein optionally one methylene group is replaced by oxygen or sulphur and wherein said C₃-C₆-alkanediyl or C₃-C₆-alkenediyl group is optionally substituted with one or two substituents selected from the group consisting of halogen, hydroxyl, C₁-C₄-alkyl and C₁-C₄-alkoxy or wherein a C₃-C₆-alkanediyl, C₃-C₆-alkenediyl or C₄-C₆-alkanedienediyl group is optionally attached to two adjacent carbon atoms of said C₃-C₆-alkanediyl or C₃-C₆-alkenediyl group forming a fused ring system.

3. The compound according to claim 1, provided that when
G represents hydrogen (a), then
A represents hydrogen or C₁-C₆-alkyl,
B represents hydrogen or C₁-C₄-alkyl,
D represents C₁-C₆-alkyl, C₃-C₆-alkenyl, C₁-C₄-alkoxy-C₂-C₃-alkyl or C₁-C₄-alkylthio-C₂-C₃-alkyl, each of which is optionally substituted with one, two or three substituents selected from the group consisting of fluorine and chlorine, or C₃-C₆-cycloalkyl optionally substituted with one or two substituents selected from the group consisting of fluorine, chlorine, C₁-C₂-alkyl, C₁-C₂-alkoxy and trifluoromethyl, or
A and D together represent a C₃-C₅-alkanediyl group optionally substituted with one or two substituents selected from the group consisting of C₁-C₂-alkyl and C₁-C₂-alkoxy, and wherein one methylene group is optionally replaced by oxygen or sulphur,
or A and D together with the atoms to which they are attached form one of the groups AD-1 to AD-10 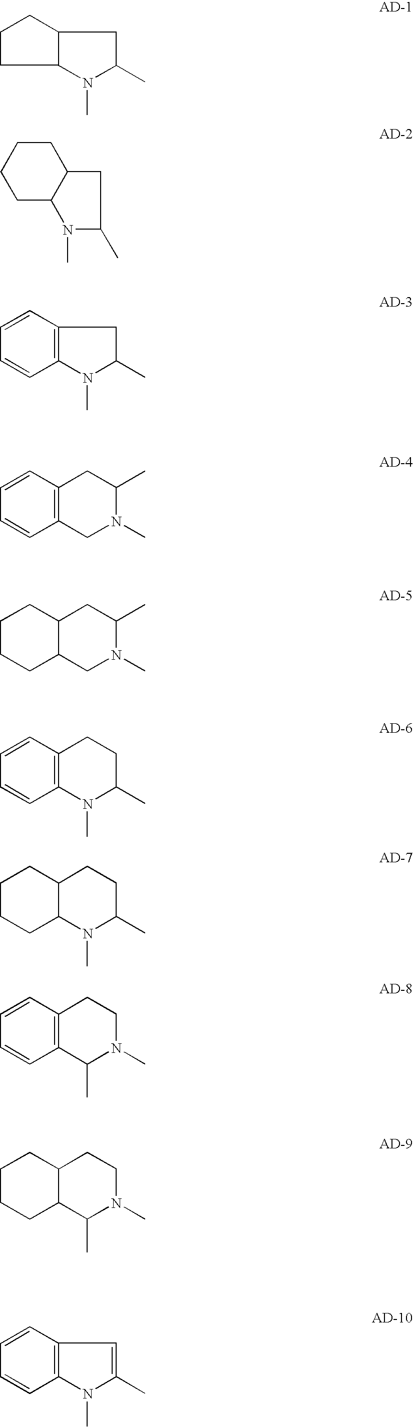
or provided that when
G represents one of the groups 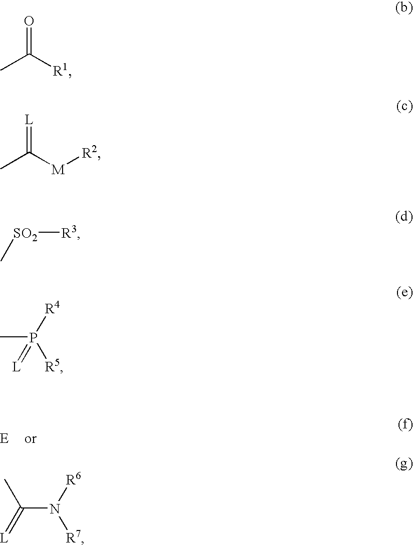
in which
E represents a metal ion equivalent or an ammonium ion,
L represents oxygen or sulphur,
M represents oxygen or sulphur,
R¹ represents C₁-C₁₀-alkyl, C₂-C₁₀-alkenyl, C₁-C₄-alkoxy-C₁-C₂-alkyl, poly-C₁-C₃-alkoxy-C₁-C₂-alkyl or C₁-C₄-alkylthio-C₁-C₂-alkyl, each of which is optionally substituted with one to five substituents selected from the group consisting of fluorine and chlorine, monosubstituted by cyano or monosubstituted by CO—R¹³, C═N—OR¹³ or CO₂R¹³, or C₃-C₆-cycloalkyl optionally substituted with one or two substituents selected from the group consisting of fluorine, chlorine, C₁-C₂-alkyl and C₁-C₂-alkoxy wherein one or two not directly adjacent methylene groups of said C₃-C₆-cycloalkyl are optionally replaced by oxygen,
phenyl or benzyl, each of which is optionally substituted with one or two substituents selected from the group consisting of fluorine, chlorine, bromine, cyano, nitro, C₁-C₄-alkyl, C₁-C₄-alkylthio, C₁-C₄-alkylsulfinyl, C₁-C₄-alkylsulfonyl, C₁-C₄-alkoxy, C₁-C₂-haloalkyl and C₁-C₂-haloalkoxy, or
pyrazolyl, thiazolyl, pyridyl, pyrimidyl, furanyl or thienyl, each of which is optionally substituted with one or two substituents selected from the group consisting of fluorine, chlorine, bromine and C₁-C₂-alkyl,
R² represents C₁-C₁₀-alkyl, C₂-C₁₀-alkenyl, C₁-C₄-alkoxy-C₂-C₄-alkyl or poly-C₁-C₄-alkoxy-C₂-C₄-alkyl, each of which is optionally substituted with one, two or three substituents selected from the group consisting of fluorine and chlorine,
C₃-C₇-cycloalkyl which is optionally monosubstituted by C₁-C₂-alkyl or C₁-C₂-alkoxy or
phenyl or benzyl, each of which is optionally substituted with one or two substituents selected from the group consisting of fluorine, chlorine, bromine, cyano, nitro, C₁-C₄-alkyl, methoxy, trifluoromethyl and trifluoromethoxy,
R³ represents C₁-C₄-alkyl which is optionally substituted with one, two or three substituents selected from the group consisting of fluorine and chlorine or phenyl or benzyl, each of which is optionally monosubstituted by fluorine, chlorine, bromine, C₁-C₄-alkyl, C₁-C₄-alkoxy, trifluoromethyl, trifluoromethoxy, cyano or nitro,
R⁴ and R⁵ independently of one another represent C₁-C₆-alkyl, C₁-C₆-alkoxy, C₁-C₆-alkylamino, di(C₁-C₆-alkyl)amino, C₁-C₆-alkylthio or C₃-C₄-alkenylthio, each of which is optionally substituted with one, two or three substituents selected from the group consisting of fluorine and chlorine, or phenyl, phenoxy or phenylthio, each of which is optionally substituted with one or two substituents selected from the group consisting of fluorine, chlorine, bromine, nitro, cyano, C₁-C₃-alkoxy, trifluoromethoxy, C₁-C₃-alkylthio, C₁-C₃-alkyl and trifluoromethyl,
R⁶ and R⁷ independently of one another represent hydrogen, C₁-C₆-alkyl, C₃-C₆-cycloalkyl, C₁-C₄-alkoxy, C₃-C₆-alkenyl or C₁-C₆-alkoxy-C₂-C₆-alkyl, wherein said C₁-C₆-alkyl, C₃-C₆-cycloalkyl, C₁-C₄-alkoxy, C₃-C₆-alkenyl or C₁-C₆-alkoxy-C₂-C₆-alkyl is optionally substituted with one, two or three substituents selected from the group consisting of fluorine and chlorine, phenyl optionally substituted with one or two substituents selected from the group consisting of fluorine, chlorine, bromine, trifluoromethyl, C₁-C₄-alkyl and C₁-C₄-alkoxy or R⁶ and R⁷ together represent a C₅-C₆-alkylene radical which is optionally mono- or disubstituted by methyl and in which optionally one methylene group is replaced by oxygen,
R¹³ represents C₁-C₄-alkyl, C₃-C₄-alkenyl, C₃-C₄-alkynyl, C₁-C₄-alkoxy-C₂-C₃-alkyl, or C₃-C₆-cycloalkyl wherein optionally one methylene group of said C₃-C₆-cycloalkyl is replaced by oxygen, then
A represents hydrogen, C₁-C₆-alkyl, C₂-C₆-alkenyl, C₁-C₄-alkoxy-C₁-C₃-alkyl or C₁-C₄-alkylthio-C₁-C₃-alkyl, wherein said C₁-C₆-alkyl, C₂-C₆-alkenyl, C₁-C₄-alkoxy-C₁-C₃alkyl, or C₁-C₄-alkylthio-C₁-C₃-alkyl is optionally substituted with one, two or three substituents selected from the group consisting of fluorine and chlorine, or C₃-C₆-cycloalkyl which is optionally substituted with one or two substituents selected from the group consisting of fluorine, chlorine, C₁-C₂-alkyl and C₁-C₂-alkoxy,
B represents hydrogen, C₁-C₄-alkyl or C₁-C₄-alkoxy-C₁-C₂-alkyl,
D represents hydrogen or
D represents C₁-C₆-alkyl, C₃-C₆-alkenyl, C₁-C₄-alkoxy-C₂-C₃-alkyl or C₁-C₄-alkylthio-C₂-C₃-alkyl, wherein said C₁-C₆-alkyl, C₃-C₆-alkenyl, C₁-C₄-alkoxy-C₂-C₃-alkyl or C₁-C₄-alkylthio-C₂-C₃-alkyl is optionally substituted with one, two or three substituents selected from the group consisting of fluorine and chlorine, or C₃-C₆-cycloalkyl optionally substituted with one or two substituents selected from the group consisting of fluorine, chlorine, C₁-C₂-alkyl, C₁-C₂-alkoxy and trifluoromethyl, provided that
A represents hydrogen or C₁-C₃-alkyl, or
A and D together represent a C₃-C₅-alkanediyl group in which optionally one methylene group is replaced by oxygen or sulphur and which is optionally substituted with one or two substituents selected from the group consisting of C₁-C₂-alkyl and C₁-C₂-alkoxy,
or A and D together with the atoms to which they are attached form one of the groups AD-1 to AD-10

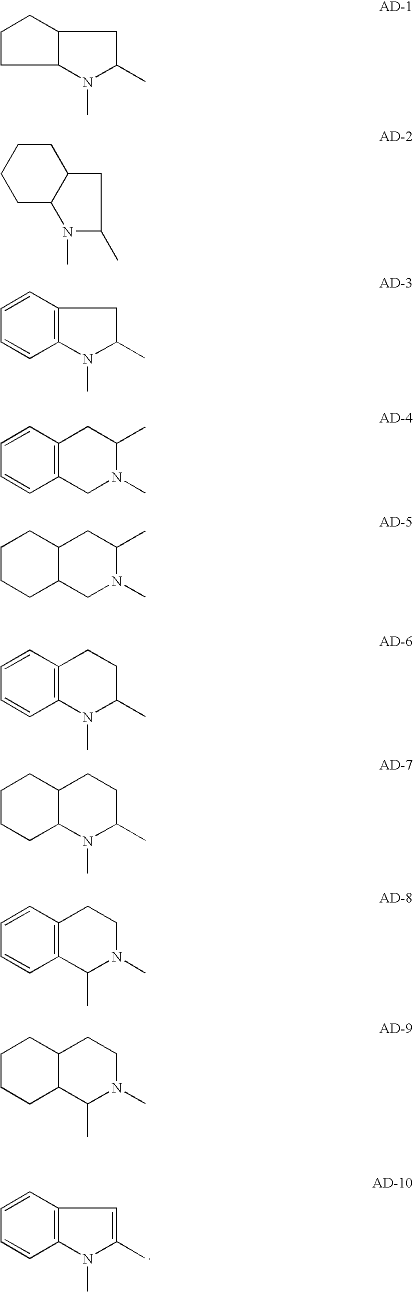

4. The compound according to claim 1, provided that when
G represents hydrogen (a), then
A represents hydrogen, methyl or ethyl,
B represents hydrogen,
D represents methyl, ethyl, n-propyl, isopropyl, n-butyl, sec-butyl, isobutyl, cyclopropyl, cyclopentyl or cyclohexyl, or
A and D together represent a C₃-C₄-alkanediyl group in which optionally one methylene group is replaced by oxygen or sulphur and which is optionally mono- or disubstituted by methyl,
or A and D together with the atoms to which they are attached form the following group: 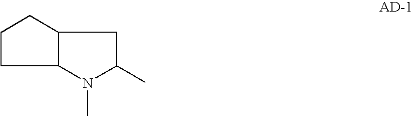
or provided that when
G represents one of the groups 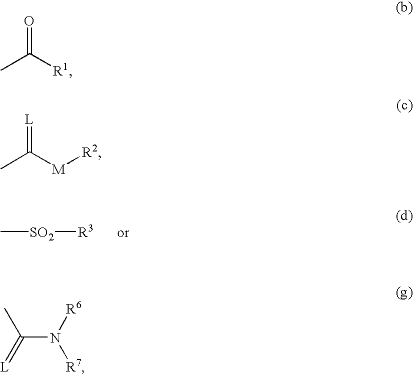
in which
L represent oxygen,
M represents oxygen or sulphur,
R¹ represents C₁-C₆-alkyl, C₂-C₆-alkenyl, C₁-C₂-alkoxy-C₁-C₂-alkyl, C₁-C₂-alkylthio-C₁-C₂-alkyl or poly-C₁-C₂-alkoxy-C₁-C₂-alkyl, each of which is optionally substituted with one, two or three substituents selected from the group consisting of fluorine and chlorine, or cyclopropyl, cyclopentyl or cyclohexyl, each of which is optionally monosubstituted by fluorine, chlorine, methyl, ethyl or methoxy,
phenyl which is optionally monosubstituted by fluorine, chlorine, bromine, cyano, nitro, methyl, ethyl, n-propyl, isopropyl, methoxy, ethoxy, methylthio, ethylthio, methylsulfinyl, ethylsulfinyl, methylsulfonyl, ethylsulfonyl, trifluoromethyl or trifluoromethoxy, or
furanyl, thienyl or pyridyl, each of which is optionally monosubstituted by chlorine, bromine or methyl,
R² represents C₁-C₈-alkyl, C₂-C₆-alkenyl, C₁-C₃-alkoxy-C₂-C₃-alkyl, cyclopentyl, cyclohexyl,
or phenyl or benzyl, wherein said phenyl or benzyl is optionally monosubstituted by fluorine, chlorine, bromine, cyano, nitro, methyl, methoxy, trifluoromethyl or trifluoromethoxy,
R³ represents C₁-C₄-alkyl which is optionally substituted with one, two or three substituents selected from the group consisting of fluorine and chlorine, or phenyl or benzyl, each of which is optionally monosubstituted by fluorine, chlorine, bromine, C₁-C₄-alkyl, C₁-C₄-alkoxy, trifluoromethyl, trifluoromethoxy, cyano or nitro,
R⁶ represents hydrogen, C₁-C₄-alkyl, C₃-C₆-cycloalkyl, allyl, or phenyl, wherein said phenyl is optionally monosubstituted by fluorine, chlorine, bromine, methyl, methoxy or trifluoromethyl,
R⁷ represents methyl, ethyl, n-propyl, isopropyl or allyl, or
R⁶ and R⁷ together represent a C₅-C₆-alkylene radical in which optionally one methylene group is replaced by oxygen, then
A represents hydrogen, methyl, ethyl, n-propyl, isopropyl, n-butyl, isobutyl, sec-butyl, tert-butyl, trifluoromethyl, cyclopropyl, cyclopentyl or cyclohexyl,
B represents hydrogen, methyl or ethyl,
D represents hydrogen or
D represents methyl, ethyl, n-propyl, isopropyl, n-butyl, sec-butyl, isobutyl, cyclopropyl, cyclopentyl or cyclohexyl, provided that
A represents hydrogen, methyl or ethyl, or
A and D together represent a C₃-C₄-alkanediyl group in which optionally one methylene group is replaced by oxygen or sulphur and which is optionally mono- or disubstituted by methyl, or
A and D together with the atoms to which they are attached form the group below:

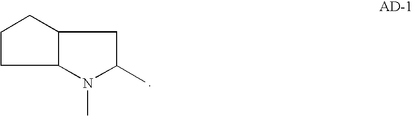

5. The compound according to claim 1, provided that when
G represents hydrogen (a), then
A represents hydrogen, methyl or ethyl,
B represents hydrogen,
D represents methyl, ethyl or cyclopropyl, or
A and D together with the atoms to which they are attached form the group below: 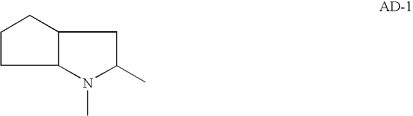
or provided that when
G represents one of the groups 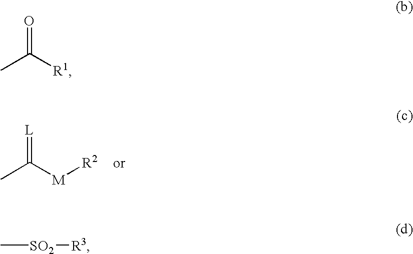
in which
L represents oxygen,
M represents oxygen,
R¹ represents C₁-C₆-alkyl or C₁-C₂-alkoxy-C₁-C₂-alkyl,
R² represents C₁-C₈-alkyl,
R³ represents C₁-C₄-alkyl, then
A represents hydrogen, methyl, ethyl, n-propyl, isopropyl or isobutyl,
B represents hydrogen, methyl or ethyl,
D represents hydrogen or
D represents methyl, ethyl or cyclopropyl, provided that
A represents hydrogen, methyl or ethyl, or
A and D together with the atoms to which they are attached form the group below:

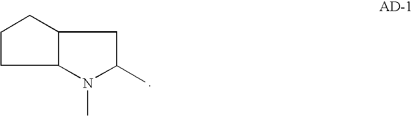

6. A process for preparing a compound of formula (I) according to claim 1, comprising
(A)
condensing intramolecularly a compound of the formula (II), 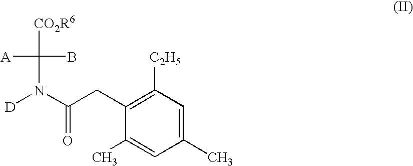
in which
A, B and D are as defined in claim 1, and
R⁸ represents alkyl,
in the presence of a diluent and in the presence of a base, to obtain a compound of the formula (I-a), 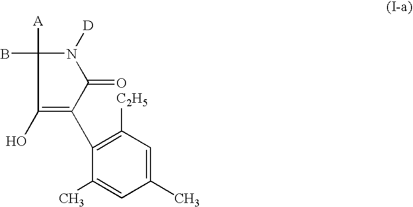
wherein A, B and D are as defined in claim 1,
(B)
reacting a compound of the formula (I-a) 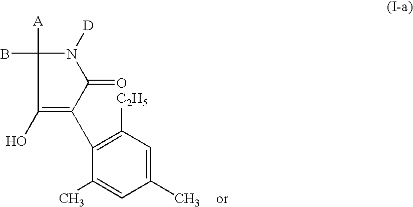
a compound of the formula (I-a′) 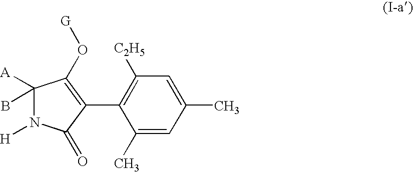
wherein A, B, and D are as defined in claim 1 and G is hydrogen,
α) with an acid halide of the formula (III), 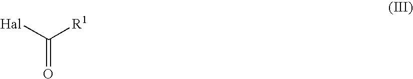
in which
R¹ is as defined in claim 1 and
Hal represents halogen, or
β) with a carboxylic anhydride of the formula (IV),
R¹—CO—O—CO—R¹ (IV)
in which
R¹ is as defined in claim 1,
optionally in the presence of a diluent and optionally in the presence of an acid binder, to obtain a compound of the formula (I-b) 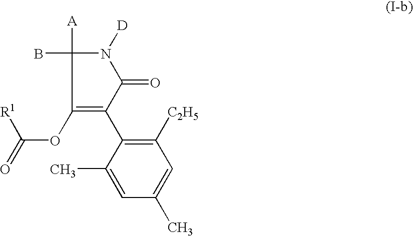
wherein A, B, D, and R¹ are as defined in claim 1,
(C)
reacting a compound of the formula (I-a) 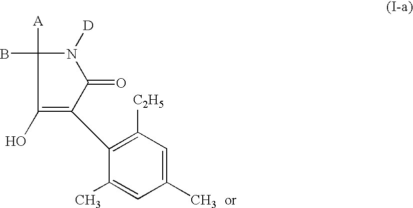
a compound of the formula (I-a′) 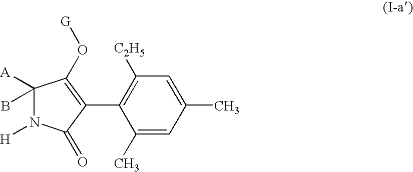
wherein A, B, and D are as defined in claim 1 and G is hydrogen,
with a chloroformic ester or a chloroformic thioester of the formula (V),
R²-M-CO—Cl (V)
in which
R² and M are as defined in claim 1,
optionally in the presence of a diluent and optionally in the presence of an acid binder, to obtain a compound of the formula (I-c) 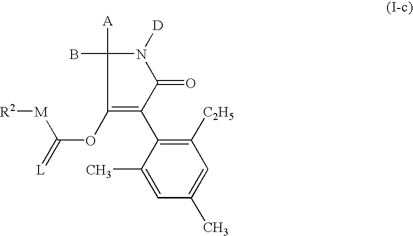
wherein A, B, D, R² and M are as defined in claim 1, and L is oxygen,
(D)
reacting a compound of the formula (I-a) 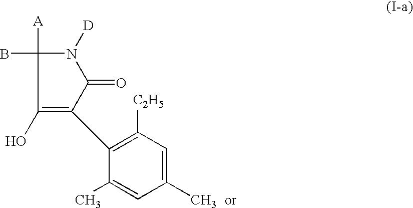
a compound of the formula (I-a′) 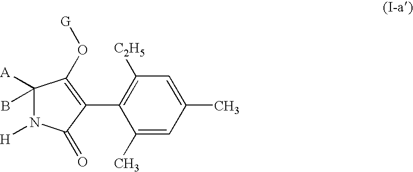
wherein A, B, and D are as defined in claim 1 and G is hydrogen,
α) with a chloromonothioformic ester or a chlorodithioformic ester of the formula (VI), 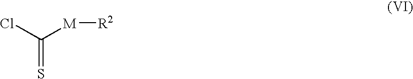
in which
M and R² are as defined in claim 1,
optionally in the presence of a diluent and optionally in the presence of an acid binder, or
β) with carbon disulphide and then with a compound of the formula (VII),
R²-Hal (VII)
in which
R² is as defined in claim 1 and
Hal represents chlorine, bromine or iodine,
optionally in the presence of a diluent and optionally in the presence of a base, to obtain a compound of the formula (I-c) 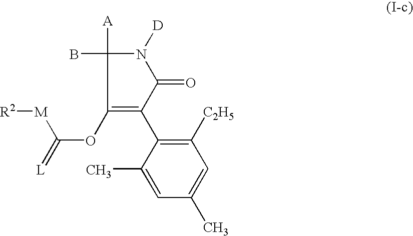
wherein A, B, D, R² and M are as defined in claim 1, and L is sulphur,
(E)
reacting a compound of the formula (I-a) 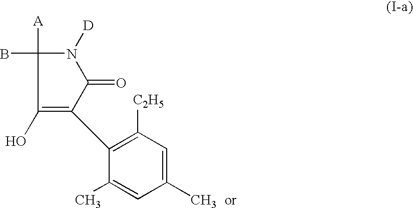
a compound of the formula (I-a′) 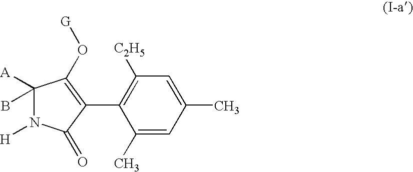
wherein A, B, and D are as defined in claim 1 and G is hydrogen, with a sulfonyl chloride of the formula (VIII),
R³—SO₂—Cl (VIII)
in which
R³ is as defined in claim 1,
optionally in the presence of a diluent and optionally in the presence of an acid binder, to obtain a compound of the formula (I-d) 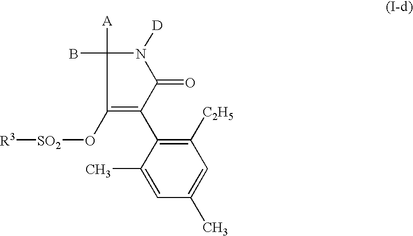
wherein A, B, D, and R³ are as defined in claim 1,
(F)
reacting a compound of the formula (I-a) 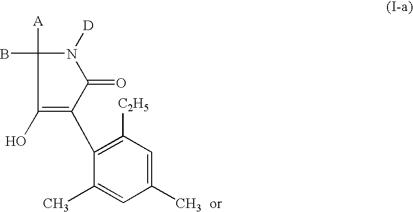
a compound of the formula (I-a′) 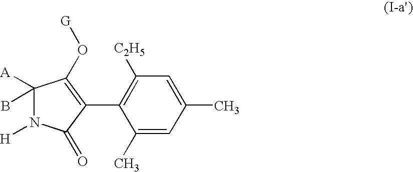
wherein A, B, and D are as defined in claim 1 and G is hydrogen, with a phosphorus compound of the formula (IX), 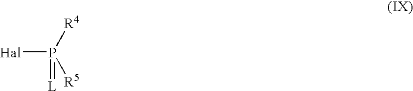
in which
L, R⁴ and R⁵ are as defined in claim 1 and
Hal represents halogen,
optionally in the presence of a diluent and optionally in the presence of an acid binder, to obtain a compound of the formula (I-e) 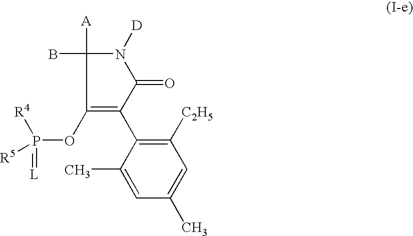
wherein A, B, D, L, R⁴, and R⁵ are as defined in claim 1,
(G)
reacting a compound of the formula (I-a) 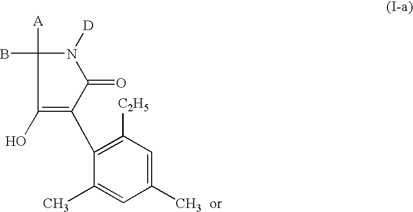
a compound of the formula (I-a′) 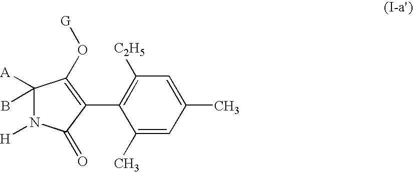
wherein A, B, and D are as defined in claim 1 and G is hydrogen, with a metal compound or an amine of the formulae (X) or (XI), respectively, 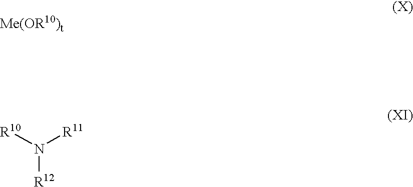
in which
Me represents a mono- or divalent metal,
t represents the number 1 or 2 and
R¹⁰, R¹¹, and R¹² independently of one another represent hydrogen or alkyl,
optionally in the presence of a diluent, to obtain a compound of the formula (I-f) 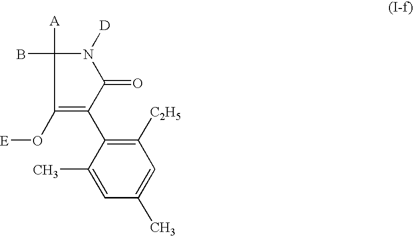
wherein A, B, D, and E are as defined in claim 1, or
(H)
reacting a compound of the formula (I-a) 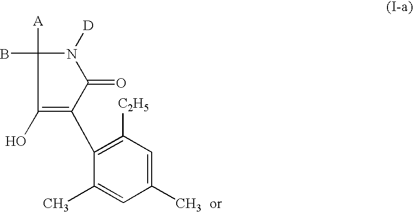
a compound of the formula (I-a′) 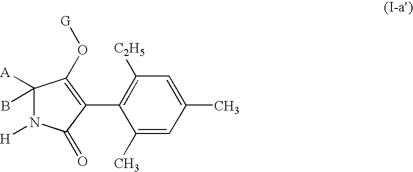
wherein A, B, and D are as defined in claim 1 and G is hydrogen,
α) with an isocyanate or an isothiocyanate of the formula (XII),
R⁶—N═C=L (XII)
in which
R⁶ and L are as defined in claim 1,
optionally in the presence of a diluent and optionally in the presence of a catalyst, or
β) with a carbamoyl chloride or a thiocarbamoyl chloride of the formula (XIII), 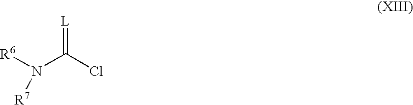
in which
L, R⁶ and R⁷ are as defined in claim 1,
optionally in the presence of a diluent and optionally in the presence of an acid binder, to obtain a compound of the formula (I-g) 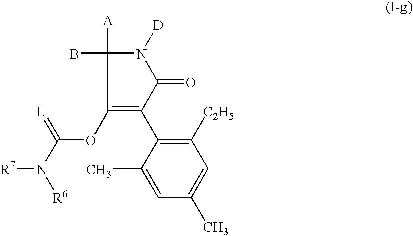
wherein A, B, D, L, R⁶, and R⁷ are as defined in claim 1.

7. (canceled)

8. A pesticide or a herbicide preparation, comprising at least one compound of the formula (I) according to claim 1.

9. A method for controlling animal pests or unwanted vegetation, comprising contacting a compound of the formula (I) according to claim 1 with pests or their habitat or unwanted vegetation.

10. (canceled)

11. A process for preparing a pesticide or a herbicide preparation, comprising mixing a compound of the formula (I) according to claim 1 with one or more extenders or surfactants, or combinations thereof.

12. A composition, comprising an effective amount of a combination of active compounds comprising
a′) at least one compound of the formula (I) according to claim 1, or
b′) at least one compound of the formula (I-a) 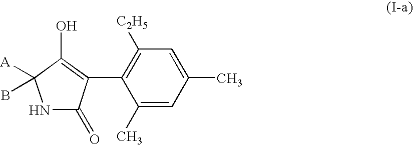
in which
A and B are as defined in claim 1 and
c′) at least one crop plant compatibility-improving compound selected from the group consisting of:
4-dichloroacetyl-1-oxa-4-azaspiro[4.5]decane (AD-67, MON-4660), 1-dichloroacetylhexahydro-3,3,8a-trimethylpyrrolo[1,2-a]pyrimidin-6(2H)-one (dicyclonon, BAS-145138), 4-dichloroacetyl-3,4-dihydro-3-methyl-2H-1,4-benzoxazine (benoxacor), 1-methylhexyl 5-chloroquinoline-8-oxyacetate (cloquintocet-mexyl, 3-(2-chlorobenzyl)-1-(1-methyl-1-phenylethyl)urea (cumyluron), α-(cyanomethoximino)phenylacetonitrile (cyometrinil), 2,4-dichlorophenoxyacetic acid (2,4-D), 4-(2,4-dichlorophenoxy)butyric acid (2,4-DB), 1-(1-methyl-1-phenylethyl)-3-(4-methylphenyl)urea (daimuron, dymron), 3,6-dichloro-2-methoxybenzoic acid (dicamba), S-1-methyl 1-phenylethyl piperidine-1-thiocarboxylate (dimepiperate), 2,2-dichloro-N-(2-oxo-2-(2-propenylamino)ethyl)-N-(2-propenyl)acetamide (DKA-24), 2,2-dichloro-N,N-di-2-propenylacetamide (dichlormid), 4,6-dichloro-2-phenylpyrimidine (fenclorim), ethyl 1-(2,4-dichlorophenyl)-5-trichloromethyl-1H-1,2,4-triazole-3-carboxylate (fenchlorazole-ethyl, phenylmethyl 2-chloro-4-trifluoromethylthiazole-5-carboxylate (flurazole), 4-chloro-N-(1,3-dioxolan-2-ylmethoxy)-α-trifluoroacetophenone oxime (fluxofenim), 3-dichloroacetyl-5-(2-furanyl)-2,2-dimethyloxazolidine (furilazole, MON-13900), ethyl 4,5-dihydro-5,5-diphenyl-3-isoxazolecarboxylate (isoxadifen-ethyl, 1-(ethoxycarbonyl)ethyl 3,6-dichloro-2-methoxybenzoate (lactidichlor), (4-chloro-o-tolyloxy)acetic acid (MCPA), 2-(4-chloro-o-tolyloxy)propionic acid (mecoprop), diethyl 1-(2,4-dichorophenyl)-4,5-dihydro-5-methyl-1H-pyrazole-3,5-dicarboxylate (mefenpyr-diethyl, 2-dichloromethyl-2-methyl-1,3-dioxolane (MG-191), 2-propenyl 1-oxa-4-azaspiro[4.5]decane-4-carbodithioate (MG-838), 1,8-naphthalic anhydride, α-(1,3-dioxolan-2-ylmethoximino)phenylacetonitrile (oxabetrinl), 2,2-dichloro-N-(1,3-dioxolan-2-ylmethyl)-N-(2-propenyl)acetamide (PPG-1292), 3-dichloroacetyl-2,2-dimethyloxazolidine (R-28725), 3-dichloroacetyl-2,2,5-trimethyloxazolidine (R-29148), 4-(4-chloro-o-tolyl)butyric acid, 4-(4-chlorophenoxy)butyric acid, diphenylmethoxyacetic acid, methyl diphenylmethoxyacetate, ethyl diphenylmethoxyacetate, methyl 1-(2-chlorophenyl)-5-phenyl-1H-pyrazole-3-carboxylate, ethyl 1-(2,4-dichlorophenyl)-5-methyl-1H-pyrazole-3-carboxylate, ethyl 1-(2,4-dichloro-phenyl)-5-isopropyl-1H-pyrazole-3-carboxylate, ethyl 1-(2,4-dichlorophenyl)-5-(1,1-dimethylethyl)-1H-pyrazole-3-carboxylate, ethyl 1-(2,4-dichlorophenyl)-5-phenyl-1H-pyrazole-3-carboxylate, ethyl 5-(2,4-dichlorobenzyl)-2-isoxazoline-3-carboxylate, ethyl 5-phenyl-2-isoxazoline-3-carboxylate, ethyl 5-(4-fluorophenyl)-5-phenyl-2-isoxazoline-3-carboxylate, 1,3-dimethylbut-1-yl 5-chloroquinoline-8-oxyacetate, 4-allyloxybutyl 5-chloroquinoline-8-oxyacetate, 1-allyloxyprop-2-yl 5-chloroquinoline-8-oxyacetate, methyl 5-chloroquinoxaline-8-oxyacetate, ethyl 5-chloroquinoline-8-oxyacetate, allyl 5-chloroquinoxaline-8-oxyacetate, 2-oxoprop-1-yl 5-chloroquinoline-8-oxyacetate, diethyl 5-chloroquinoline-8-oxymalonate, diallyl 5-chloroquinoxaline-8-oxymalonate, diethyl 5-chloroquinoline-8-oxymalonate, 4-carboxychroman-4-ylacetic acid (AC-304415), 4-chlorophenoxyacetic acid, 3,3′-dimethyl-4-methoxybenzophenone, 1-bromo-4-chloromethylsulphonylbenzene, 1-[4-(N-2-methoxybenzoylsulphamoyl)phenyl]-3-methylurea (also known as N-(2-methoxybenzoyl)-4-[(methylaminocarbonyl)amino]benzenesulphonamide), 1-[4-(N-2-methoxybenzoylsulphamoyl)phenyl]-3,3-dimethylurea, 1-[4-(N-4,5-dimethylbenzoylsulphamoyl)phenyl]-3-methylurea, 1-[4-(N-naphthylsulphamoyl)phenyl]-3,3-dimethylurea, and N-(2-methoxy-5-methylbenzoyl)-4-(cyclopropylaminocarbonyl)benzenesulphonamide,
or selected from a group consisting of a compound of the general formula (IIa) 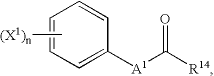
a compound of the general formula (IIb) 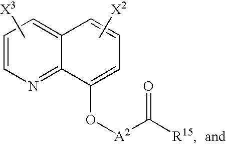
a compound of the formula (IIc) 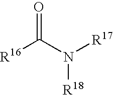
where
n represents a number from 0 to 5,
A¹ represents one of the following divalent heterocyclic groups 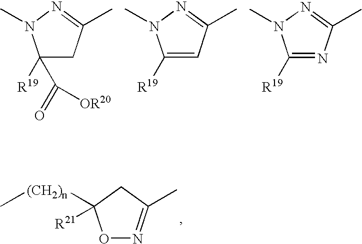
wherein n is as defined above,
A² represents alkanediyl having 1 or 2 carbon atoms optionally substituted with one or more substituents selected from the grouo consisting of C₁-C₄-alkyl and C₁-C₄-alkoxycarbonyl,
R¹⁴ represents hydroxy, mercapto, amino, C₁-C₆-alkoxy, C₁-C₆-alkylthio, C₁-C₆-alkylamino or di-(C₁-C₄-alkyl)amino,
R¹⁵ represents hydroxy, mercapto, amino, C₁-C₇-alkoxy, C₁-C₆-alkylthio, C₁-C₆-alkylamino or di-(C₁-C₄-alkyl)-amino,
R¹⁶ represents C₁-C₄-alkyl optionally substituted with one or more substituents selected from the group consisting of fluorine, chlorine and bromine,
R¹⁷ represents hydrogen, C₁-C₆-alkyl, C₂-C₆-alkenyl or C₂-C₆-alkynyl optionally substituted with one or more substituents selected from the group consisting of fluorine, chlorine and bromine, C₁-C₄-alkoxy-C₁-C₄-alkyl, dioxolanyl-C₁-C₄-alkyl, furyl, furyl-C₁-C₄-alkyl, thienyl, thiazolyl, piperidinyl, phenyl optionally substituted with one or more substituents selected from the group consisting of fluorine, chlorine and bromine, or C₁-C₄-alkyl-substituted phenyl,
R¹⁸ represents hydrogen, C₁-C₆-alkyl, C₂-C₆-alkenyl or C₂-C₆-alkynyl in each case optionally substituted with one or more substituents selected from the group consisting of fluorine, chlorine, and bromine, C₁-C₄-alkoxy-C₁-C₄-alkyl, dioxolanyl-C₁-C₄-alkyl, furyl, furyl-C₁-C₄-alkyl, thienyl, thiazolyl, piperidinyl, or phenyl optionally substituted with one or more substituents selected from the group consisting of fluorine, chlorine and bromine, or C₁-C₄-alkyl-substituted phenyl, or R¹⁸ together with R¹⁷ represents C₃-C₆-alkanediyl or C₂-C₅-oxaalkanediyl, each of which is optionally substituted by C₁-C₄-alkyl, phenyl, furyl, a fused benzene ring or by two substituents which, together with the C atom to which they are attached, form a 5- or 6-membered carbocycle,
R¹⁹ represents hydrogen, cyano, or halogen, or C₁-C₄-alkyl, C₃-C₆-cycloalkyl or phenyl in each case optionally substituted with one or more substituents selected from the group consisting of fluorine, chlorine and bromine,
R²⁰ represents hydrogen, or optionally hydroxy-, cyano-, halogen- or C₁-C₄-alkoxy-substituted C₁-C₆-alkyl, C₃-C₆-cycloalkyl or tri(C₁-C₄-alkyl)silyl,
R²¹ represents hydrogen, cyano, or halogen, or C₁-C₄-alkyl, C₃-C₆-cycloalkyl or phenyl in each case optionally substituted with one or more substituents selected from the group consisting of fluorine, chlorine and bromine,
X¹ represents nitro, cyano, halogen, C₁-C₄-alkyl, C₁-C₄-haloalkyl, C₁-C₄-alkoxy or C₁-C₄-haloalkoxy,
X² represents hydrogen, cyano, nitro, halogen, C₁-C₄-alkyl, C₁-C₄-haloalkyl, C₁-C₄-alkoxy or C₁-C₄-haloalkoxy,
X³ represents hydrogen, cyano, nitro, halogen, C₁-C₄-alkyl, C₁-C₄-haloalkyl, C₁-C₄-alkoxy or C₁-C₄-haloalkoxy,
or selected from the group consisting of a compound of the general formula (IId) 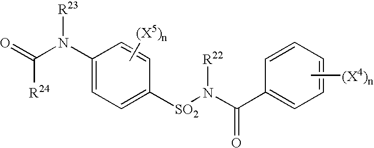
and a compound of the general formula (IIe) 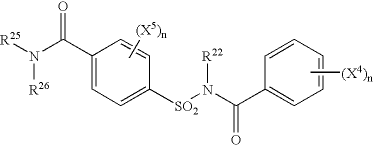
where
n represents a number from 0 to 5,
R²² represents hydrogen or C₁-C₄-alkyl,
R²³ represents hydrogen or C₁-C₄-alkyl,
R²⁴ represents hydrogen, in each case optionally cyano-, halogen- or C₁-C₄-alkoxy- substituted C₁-C₆-alkyl, C₁-C₆-alkoxy, C₁-C₆-alkylthio, C₁-C₆-alkylamino or di-(C₁-C₄-alkyl)amino, or in each case optionally cyano-, halogen- or C₁-C₄-alkyl- substituted C₃-C₆-cycloalkyl, C₃-C₆-cycloalkyloxy, C₃-C₆-cycloalkylthio or C₃-C₆-cycloalkylamino,
R²⁵ represents hydrogen, optionally cyano-, hydroxy-, halogen- or C₁-C₄-alkoxy-substituted C₁-C₆-alkyl, in each case optionally cyano- or halogen- substituted C₃-C₆-alkenyl or C₃-C₆-alkynyl, or optionally cyano-, halogen- or C₁-C₄-alkyl-substituted C₃-C₆-cycloalkyl,
R²⁶ represents hydrogen, optionally cyano-, hydroxy-, halogen- or C₁-C₄-alkoxy-substituted C₁-C₆-alkyl, in each case optionally cyano- or halogen- substituted C₃-C₆-alkenyl or C₃-C₆-alkynyl, optionally cyano-, halogen- or C₁-C₄-alkyl-substituted C₃-C₆-cycloalkyl, or optionally nitro-, cyano-, halogen-, C₁-C₄-alkyl-, C₁-C₄-haloalkyl, C₁-C₄-alkoxy- or C₁-C₄-haloalkoxy-substituted phenyl, or R²⁶ together with R²⁵ in each case represent optionally C₁-C₄-alkyl-substituted C₂-C₆-alkanediyl or C₂-C₅-oxaalkanediyl,
X⁴ represents nitro, cyano, carboxyl, carbamoyl, formyl, sulphamoyl, hydroxy, amino, halogen, C₁-C₄-alkyl, C₁-C₄-haloalkyl, C₁-C₄-alkoxy or C₁-C₄-haloalkoxy, and
X⁵ represents nitro, cyano, carboxyl, carbamoyl, formyl, sulphamoyl, hydroxy, amino, halogen, C₁-C₄-alkyl, C₁-C₄-haloalkyl, C₁-C₄-alkoxy or C₁-C₄-haloalkoxy.

13. The composition according to claim 12, where the crop plant compatibility-improving compound is selected from the group consisting of
cloquintocet-mexyl, fenchlorazole-ethyl, isoxadifen-ethyl, mefenpyr-diethyl, furilazole, fenclorim, cumyluron, dymron,

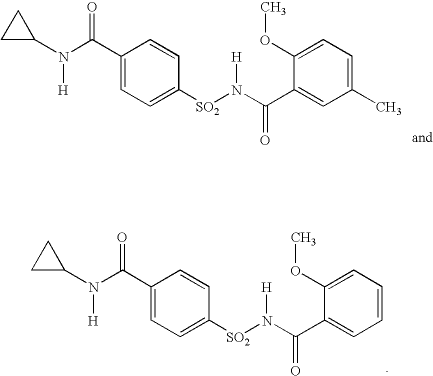

14. The composition according to claim 12 or 13 where the crop plant compatibility-improving compound is cloquintocet-mexyl or mefenpyr-diethyl.

15. A method for controlling unwanted vegetation, comprising contacting a composition according to claim 12 with the unwanted vegetation.

16. (canceled)

17. A compound of the formula (II) 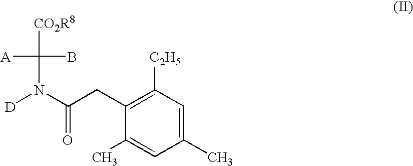
in which
A represents hydrogen in each case optionally halogen-substituted alkyl, alkenyl, alkoxyalkyl or alkylthioalkyl or optionally substituted cycloalkyl,
B represents hydrogen, alkyl or alkoxyalkyl,
D represents in each case an optionally substituted radical selected from the group consisting of alkyl, alkenyl, alkynyl, alkoxyalkyl, alkylthioalkyl, and cycloalkyl, or
A and D together with the atoms to which they are attached form a saturated or unsaturated cycle which optionally contains at least one heteroatom in the A,D moiety and which is unsubstituted or substituted in the A,D moiety, and
R⁸ represents alkyl.

18. A compound of the formula (XVI) 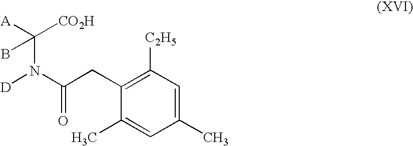
in which
A represents hydrogen, in each case optionally halogen-substituted alkyl, alkenyl, alkoxyalkyl or alkylthioalkyl or optionally substituted cycloalkyl,
B represents hydrogen, alkyl or alkoxyalkyl,
D represents in each case an optionally substituted radical selected from the group consisting of alkyl, alkenyl, alkynyl, alkoxyalkyl, alkylthioalkyl, and cycloalkyl, or
A and D together with the atoms to which they are attached form a saturated or unsaturated cycle which optionally contains at least one heteroatom in the A,D moiety and which is unsubstituted or substituted in the A,D moiety.

19. A process for preparing 2-ethyl-4,6-dimethylphenylacetic acid, comprising reacting 2-ethyl-4,6-dimethylbromobenzene with tert-butyl acetate optionally in the presence of a base, a phosphine ligand, a palladium compound and a diluent, and subsequently contacting with an acid.
